(19) United States
(12) Patent Application Publication
(10) Pub. No.: US 2023/0210825 A1
(43) Pub. Date: Jul. 6, 2023

(54) METHODS AND COMPOSITIONS FOR TREATING SUBJECTS WITH METABOLIC DISORDERS AND CORONAVIRUS INFECTIONS

(71) Applicant: CIRIUS THERAPEUTICS, INC., San Diego, CA (US)

(72) Inventor: Gerard R. COLCA, San Diego, CA (US)

(21) Appl. No.: 18/046,848

(22) Filed: Oct. 14, 2022

Related U.S. Application Data
(63) Continuation of application No. PCT/US2021/027803, filed on Apr. 16, 2021.
(60) Provisional application No. 63/011,926, filed on Apr. 17, 2020.

(51) Int. Cl. A61K 31/426; A61P 31/14; A61P 3/10

(52) U.S. Cl. CPC A61K 31/426; A61P 31/14; A61P 3/10

(57) ABSTRACT

Provided herein are thiazolidinedione analogues that are useful for treating metabolic disorders and/or coronavirus infections. In some embodiments, the metabolic disorder includes insulin resistance, diabetes, or prediabetes. In some embodiments, the coronavirus is COVID-19.

### CROSS-REFERENCE

This is a continuation of International Application No. PCT/US2021/027803, filed Apr. 16, 2021, which claims the benefit of U.S. Provisional Patent Application No. 63/011,926, filed Apr. 17, 2020, each of which are incorporated herein by reference in their entirety.

### BACKGROUND

Peroxisome Proliferator Activated Receptors (PPARs), which are members of the nuclear hormone receptor super family, are ligand-activated transcription factors that regulate gene expression. PPARs have been implicated in autoimmune diseases and other diseases (e.g., diabetes mellitus, cardiovascular disease, gastrointestinal disease, and Alzheimer's disease). First generation thiazolidinediones approved for treatment of type II diabetes are direct activators of the PPAR gamma subtype. Newer agents work by modifying the activity of the mitochondrial pyruvate carrier.

The mitochondrial pyruvate carrier (MPC) comprises two proteins, MPC1 and MPC2, that form a carrier complex in the inner mitochondrial membrane. Transport into the mitochondrial matrix is required for pyruvate metabolism and critical for a number of metabolic pathways. Modulation of MPC indirectly affects PPAR networks.

### BRIEF SUMMARY OF THE INVENTION

Compounds selective for modulation of the MPC are uniquely beneficial as treatments for acute respiratory infections especially in the context of metabolic impairment. Disclosed herein, in some embodiments, are methods of treatment comprising administering to a subject in need thereof: a therapeutically effective amount of a compound of structural Formula (I):

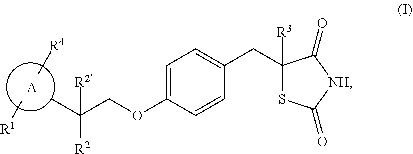

or a pharmaceutically acceptable salt thereof, wherein: R¹ is hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and wherein the subject has a metabolic disorder and a coronavirus infection. Some embodiments include identifying the subject as having the metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises hyperglycemia. Some embodiments include identifying the subject as having the coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, administration of a direct PPARγ agonist such as pioglitazone is contraindicated. Some embodiments include determining that administration of the direct PPARγ agonist is contraindicated. In some embodiments, the administration of the compound of structural Formula (I) does not result in adverse respiratory side-effects such as fluid accumulation, or exacerbate the respiratory disorder.

Disclosed herein, in some embodiments, are methods of treating a respiratory disorder, comprising administering to a subject in need thereof, and having or being suspected of having the respiratory disorder: a therapeutically effective amount of a compound of structural Formula (I):

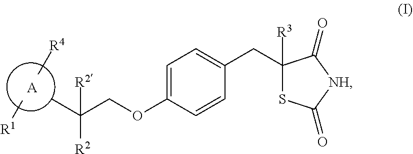

or a pharmaceutically acceptable salt thereof, wherein: R¹ is hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is hydrogen, halogen, substituted or unsubstituted alkyl, or —OR^{4A}; A is phenyl; and R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Some embodiments include identifying the subject as having or being suspected of having a metabolic disorder. In some embodiments, the subject has the metabolic disorder. Some embodiments include identifying the subject as having the metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises hyperglycemia. Some embodiments include identifying the subject as having or being suspected of having the respiratory disorder. In some embodiments, the subject has the respiratory disorder. Some embodiments include identifying the subject as having the respiratory disorder. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, administration of a direct PPARγ agonist such as pioglitazone is contraindicated. Some embodiments include determining that administration of the direct PPARγ agonist is contraindicated.

Disclosed herein, in some embodiments, are methods of treating a metabolic disorder, comprising administering to a subject in need thereof, and having or being suspected of having the metabolic disorder: a therapeutically effective amount of a compound of structural Formula (I):

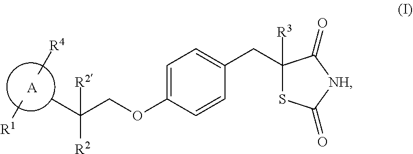

or a pharmaceutically acceptable salt thereof, wherein: R¹ is hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is hydrogen, halogen, substituted or unsubstituted alkyl, or —OR^{4A}; A is phenyl; and R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Some embodiments include identifying the subject as having or being suspected of having the metabolic disorder. In some embodiments, the subject has the metabolic disorder. Some embodiments include identifying the subject as having the metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises hyperglycemia. Some embodiments include identifying the subject as having or being suspected of having a respiratory disorder. In some embodiments, the subject has or is suspected of having a respiratory disorder. Some embodiments include identifying the subject as having the respiratory disorder. In some embodiments, the subject has the respiratory disorder. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, administration of a direct PPARγ agonist such as pioglitazone is contraindicated. Some embodiments include determining that administration of the direct PPARγ agonist is contraindicated. In some embodiments, the administration of the compound of structural Formula (I) does not result in adverse respiratory side-effects such as fluid accumulation, or exacerbate the respiratory disorder.

Disclosed herein, in some embodiments, are methods of inhibiting a mitochondrial pyruvate carrier (MPC) in a subject having or suspected of having a respiratory disorder, comprising contacting the MPC with a compound of structural Formula (I):

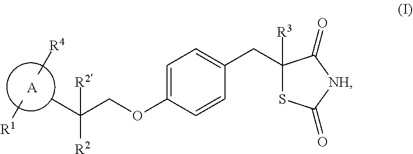

or a pharmaceutically acceptable salt thereof, wherein: R¹ is hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is hydrogen, halogen, substituted or unsubstituted alkyl, or —OR^{4A}; A is phenyl; and R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Some embodiments include identifying the subject as having or being suspected of having a metabolic disorder. In some embodiments, the subject has the metabolic disorder. Some embodiments include identifying the subject as having the metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises hyperglycemia. Some embodiments include identifying the subject as having or being suspected of having the respiratory disorder. In some embodiments, the subject has the respiratory disorder. Some embodiments include identifying the subject as having the respiratory disorder. In some embodiments, the subject is an adult human. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, contact with a direct PPARγ agonist such as pioglitazone is contraindicated. Some embodiments include determining that contact with the direct PPARγ agonist is contraindicated. In some embodiments, the contact with the compound of structural Formula (I) does not result in adverse respiratory side-effects such as fluid accumulation, or exacerbate the respiratory disorder.

Disclosed herein, in some embodiments, are methods of improving or increasing glucose tolerance and/or insulin sensitivity, comprising administering to a subject in need thereof, a therapeutically effective amount of a compound of structural Formula (I):

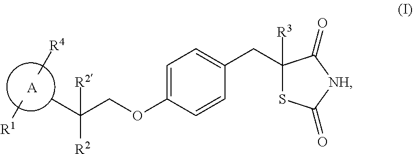

or a pharmaceutically acceptable salt thereof, wherein: R¹ is hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and wherein the subject has or is suspected of having a respiratory disorder. Some embodiments include identifying the subject as having or being suspected of having insulin resistance. In some embodiments, the subject has insulin resistance. Some embodiments include identifying the subject as having insulin resistance. In some embodiments, the subject has hyperinsulinemia. Some embodiments include identifying the subject as having or being suspected of having glucose intolerance. In some embodiments, the subject has glucose intolerance. Some embodiments include identifying the subject as having glucose intolerance. In some embodiments, the subject has hyperglycemia. In some embodiments, the subject has diabetes or is prediabetic. In some embodiments, the diabetes comprises diabetes mellitus type II. Some embodiments include identifying the subject as having or being suspected of having the respiratory disorder. In some embodiments, the subject has the respiratory disorder. Some embodiments include identifying the subject as having the respiratory disorder. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, administration of a direct PPARγ agonist such as pioglitazone is contraindicated. Some embodiments include determining that administration of the direct PPARγ agonist is contraindicated. In some embodiments, the administration of the compound of structural Formula (I) does not result in adverse respiratory side-effects such as fluid accumulation, or exacerbate the respiratory disorder.

Disclosed herein, in some embodiments, are methods or compositions relating to a compound of structural Formula (I):

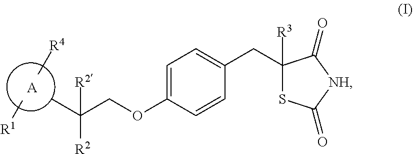

or a pharmaceutically acceptable salt thereof, wherein: R¹ is hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R³ is hydrogen. In some embodiments, R⁴ is: hydrogen, methyl, or —OR^{4A}; and R^{4A} is methyl, ethyl, isopropyl, —CHF₂, or —CF₃. In some embodiments, R⁴ is hydrogen. In some embodiments, R¹ is: hydrogen, halogen, or —OR^{1A}; and R^{1A} is substituted or unsubstituted alkyl. In some embodiments, R¹ is hydrogen. In some embodiments, R¹ is halogen. In some embodiments, R¹ is —OR^{1A} and R^{VA} is substituted or unsubstituted alkyl. In some embodiments, R¹ is attached to the para or meta position of the phenyl. In some embodiments, R¹ is attached to the meta position of the phenyl. In some embodiments, R¹ is —F or —Cl. In some embodiments, R¹ is attached to the ortho or meta position of the phenyl. In some embodiments, R¹ is attached to the meta position of the phenyl. In some embodiments, R^{VA} is substituted or unsubstituted C₁-C₃alkyl. In some embodiments, R^{1A} is —CHF₂ or —CF₃. In some embodiments, R^{2′} is hydrogen. In some embodiments, R² is hydroxyl. In some embodiments, R² and R^{2′} are joined to form oxo. In some embodiments, the compound of structural Formula (I) is:

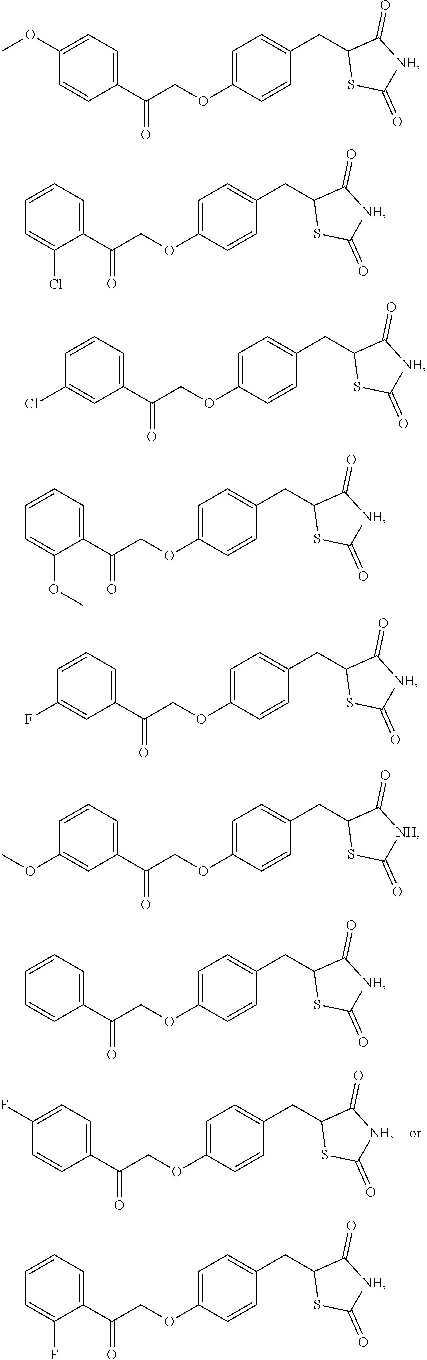

or a pharmaceutically acceptable salt thereof. In some embodiments, the compound of structural Formula (I) is:

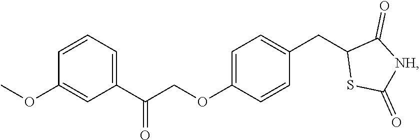 [SMILES: COc1cccc(C(=O)COc2ccc(CC3SC(=O)NC3=O)cc2)c1]

or a pharmaceutically acceptable salt thereof. In some embodiments, the compound inhibits a mitochondrial pyruvate carrier (MPC) in the subject. In some embodiments, the compound has reduced PPAR binding, as compared to one or more direct PPARγ agonists such as pioglitazone.

Disclosed herein, in some embodiments, are methods of reducing alanine transaminase (ALT) and/or aspartate aminotransferase (AST) in a subject diagnosed with a respiratory disorder, the method comprising administering to the subject a therapeutically effective amount of a compound of structural Formula (I):

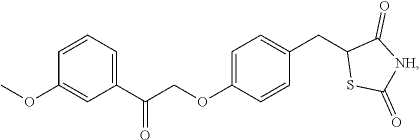 [SMILES: COc1cccc(C(=O)COc2ccc(CC3SC(=O)NC3=O)cc2)c1]

or a pharmaceutically acceptable salt thereof. Some embodiments include identifying the subject as having or being suspected of having a metabolic disorder. In some embodiments, the subject has the metabolic disorder. Some embodiments include identifying the subject as having the metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises hyperglycemia. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, administration of a direct PPARγ agonist such as pioglitazone is contraindicated. Some embodiments include determining that administration of the direct PPARγ agonist is contraindicated. In some embodiments, the administration of the compound of structural Formula (I) does not result in adverse respiratory side-effects such as fluid accumulation, or exacerbate the respiratory disorder.

Disclosed herein, in some embodiments, are methods of reducing hemoglobin A1c (HbA1c) in a subject diagnosed with diabetes and having or suspected of having a respiratory disorder, the method comprising administering to the subject a therapeutically effective amount of a compound of structural Formula (I):

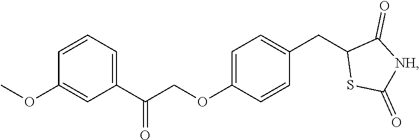 [SMILES: COc1cccc(C(=O)COc2ccc(CC3SC(=O)NC3=O)cc2)c1]

or a pharmaceutically acceptable salt thereof. In some embodiments, the diabetes comprises diabetes mellitus type II. In some embodiments, the subject has insulin resistance. In some embodiments, the subject has hyperinsulinemia. In some embodiments, the subject has glucose intolerance. In some embodiments, the subject has hyperglycemia. Some embodiments include identifying the subject as having or being suspected of having the respiratory disorder. In some embodiments, the subject has the respiratory disorder. Some embodiments include identifying the subject as having the respiratory disorder. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, administration of a direct PPARγ agonist such as pioglitazone is contraindicated. Some embodiments include determining that administration of the direct PPARγ agonist is contraindicated. In some embodiments, the administration of the compound of structural Formula (I) does not result in adverse respiratory side-effects such as fluid accumulation, or exacerbate the respiratory disorder.

Disclosed herein, in some embodiments, are methods of inhibiting cellular mitochondrial pyruvate carrier (MPC) with reduced PPARγ agonism, as compared to one or more direct PPARγ agonists such as pioglitazone, in a subject having or suspected of having a respiratory disorder, the method comprising administering to the subject a therapeutically effective amount of a compound of structural Formula (I):

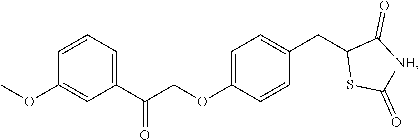 [SMILES: COc1cccc(C(=O)COc2ccc(CC3SC(=O)NC3=O)cc2)c1]

or a pharmaceutically acceptable salt thereof. Some embodiments include identifying the subject as having or being suspected of having a metabolic disorder. In some embodiments, the subject has the metabolic disorder. Some embodiments include identifying the subject as having the metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises hyperglycemia. Some embodiments include identifying the subject as having or being suspected of having the respiratory disorder. In some embodiments, the subject has the respiratory disorder. Some embodiments include identifying the subject as having the respiratory disorder. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, administration of a direct PPARγ agonist such as pioglitazone is contraindicated. Some embodiments include determining that administration of the direct PPARγ agonist is contraindicated. In some embodiments, the administration of the compound of structural Formula (I) does not result in adverse respiratory side-effects such as fluid accumulation, or exacerbate the respiratory disorder.

Disclosed herein, in some embodiments, are methods or compositions relating to a compound of structural Formula (I). In some embodiments, the compound of structural Formula (I), or a pharmaceutically acceptable salt thereof, is administered orally. In some embodiments, the compound of structural Formula (I), or a pharmaceutically acceptable salt thereof, is formulated as a tablet or capsule. In some embodiments, the therapeutically effective amount of the compound comprises a dosage amount of about 62.5 milligrams (mg), about 125 mg, or about 250 mg. In some embodiments, the therapeutically effective amount of a compound of structural Formula (I) is a single dose amount of about 125 mg or 250 mg. In some embodiments, the compound of structural Formula (I), or a pharmaceutically acceptable salt thereof, is administered in a dose of from about 60 mg to about 250 mg. In some embodiments, the compound of structural Formula (I), or a pharmaceutically acceptable salt thereof, is administered daily. In some embodiments, the compound of structural Formula (I), or a pharmaceutically acceptable salt thereof, is administered once daily. In some embodiments, the pharmaceutically acceptable salt is a potassium salt. In some embodiments, administering to a subject in need thereof a therapeutically effective amount of a compound of structural Formula (I) does not result in edema in the subject. In some embodiments, the compound inhibits a mitochondrial pyruvate carrier (MPC) in the subject.

Disclosed herein, in some embodiments, are pharmaceutical compositions comprising a dosage amount of between about 60 milligrams (mg) and about 250 mg of the compound of structural Formula (I):

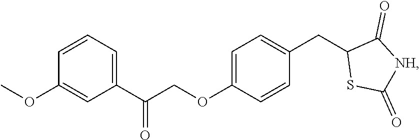 [SMILES: COc1cccc(C(=O)COc2ccc(CC3SC(=O)NC3=O)cc2)c1]

or a pharmaceutically acceptable salt thereof, for use in a subject diagnosed with a respiratory disorder. In some embodiments, the dosage amount comprises about 62.5 mg. In some embodiments, the dosage amount comprises about 125 mg. In some embodiments, the dosage amount comprises about 250 mg. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). Some embodiments include method of treating or preventing a metabolic disorder, the method comprising administering the pharmaceutical composition to a subject in need thereof, and having the respiratory disorder. In some embodiments, the subject suffers from a metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises hyperglycemia. In some embodiments, the compound inhibits a mitochondrial pyruvate carrier (MPC) in the subject. In some embodiments, administration of a direct PPARγ agonist such as pioglitazone is contraindicated. Some embodiments include determining that administration of the direct PPARγ agonist is contraindicated. In some embodiments, the administration of the compound of structural Formula (I) does not result in adverse respiratory side-effects such as fluid accumulation, or exacerbate the respiratory disorder.

Disclosed herein, in some embodiments, are methods of controlling glycemia in a subject without administering insulin to the subject, the method comprising administering to the subject a therapeutically effective amount of a compound of structural Formula (I):

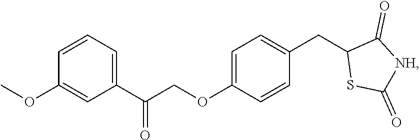 [SMILES: COc1cccc(C(=O)COc2ccc(CC3SC(=O)NC3=O)cc2)c1]

or a pharmaceutically acceptable salt thereof. In some embodiments, the administration reduces a circulating glucose level in the subject. In some embodiments, the glucose level is determined in a sample from the subject. In some embodiments, the sample is a blood sample, a serum sample, or a plasma sample. In some embodiments, the circulating glucose level is reduced compared to a control patient, or compared to a baseline circulating glucose level in the patient. In some embodiments, the circulating glucose level is reduced by at least 10%. Some embodiments include identifying the subject as having or being suspected of having a metabolic disorder. In some embodiments, the subject has the metabolic disorder. Some embodiments include identifying the subject as having the metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises hyperglycemia. Some embodiments include identifying the subject as having or being suspected of having a respiratory disorder. In some embodiments, the subject has the respiratory disorder. Some embodiments include identifying the subject as having the respiratory disorder. In some embodiments, the respiratory disorder comprises a respiratory infection. In some embodiments, the respiratory infection comprises a coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). Some embodiments include determining that administration of a PPARγ agonist to the subject is contraindicated.

### BRIEF DESCRIPTION OF THE DRAWINGS

FIG. 1. Is a graphic representation of the data of the relative binding affinities of compound MSDC-0602, the metabolite of MSDC-0602, and the two insulin sensitizers rosiglitazone and pioglitazone with PPARy.

FIG. 2. Is a graphic representation of the data of the relative binding affinities of compound MSDC-0602, the metabolite of MSDC-0602, and the two insulin sensitizers rosiglitazone and pioglitazone with the MPC.

FIG. 3. Is a graphic representation of a mechanism of effects of some compounds described herein to improve response to respiratory infection through modulation of the MPC, in accordance with some embodiments. FIG. 3 demonstrates, amongst other things, the following:
Excess metabolism drives a program of increased storage and insulin resistance
Slowing pyruvate entry rebalances metabolic signals, reversing these signals
Downstream pathways include nutrient sensors and transcriptional networks
Pyruvate carboxylase (PC) and pyruvate dehydrogenase complexes (PDH) act on mitochondrial pyruvate maintaining carbon flow in the TCA (citric acid cycle)

FIG. 4. Is a graphic showing how some metabolic effects such as MPC inhibition and/or reducing mTOR activation may prevent, decrease, or affect viral entry and/or replication in a host cell when a subject has a respiratory infection.

FIG. 5. Is a graphic representation of a mechanism in accordance with some embodiments. FIG. 5 demonstrates, amongst other things, the following:

Aspects that Favor pro-inflammatory M1 mechanisms
Dysmetabolism (insulin resistance), >mTOR activation
>Viral infection and replication
Exaggerated inflammatory response
Reduced repair
Hypoxia

Aspects that Favor repair/clearance M2 mechanisms
Insulin sensitive (less mTOR activation) or +MPC inhibitor
<Viral infection and replication
Reduced inflammatory response
Increased tissue repair
Less Hypoxia

FIGS. 6A-6C. Show the comparative results from diet-induced obese (60% HF diet) LS-MPC2−/− and WT (fl/fl) mice: Body weight (FIG. 6A); Blood glucose levels (FIG. 6B); Blood glucose AUC (FIG. 6C).

FIGS. 7A-7C. Show the results after a single dose of MSDC-0602 given to LS-MPC2−/− and WT (fl/fl) mice. Blood glucose levels (FIG. 7A); Blood glucose Area Under the Curve (AUC) (FIG. 7B); Plasma insulin levels (FIG. 7C).

FIGS. 8A-8B. Show the results after a single dose of MSDC-0602 given to LS-MPC2−/− and WT (fl/fl) mice. Plasma ALT concentrations (FIG. 8A); Gene expression for markers of liver injury (FIG. 8B).

FIG. 9. A schematic representation of the effects of hepatocyte metabolism on exosome communication with stellate cells.

FIGS. 10A-10I. Show the levels of serum miRNAs after treatment of mice with MSDC-0602: mmu-miR-29c-3p (FIG. 10A); mmu-miR-802-3p (FIG. 10B); mmu-miR-802-5p (FIG. 10C); mmu-miR-127-3p (FIG. 10D); mmu-miR-129-2-3p (FIG. 10E); mmu-miR-615-3p (FIG. 10F); mmu-miR-129-5p (FIG. 10G); mmu-miR-205-5p (FIG. 10H); mmu-miR-341-3p (FIG. 10I). (*FDR<0.05 vs LF; † FDR<0.05 vs HTF-C.)

FIG. 11. Show pathology of a metabolic disorder after 19 weeks of high fat, high cholesterol, and high sugar diet in mice, as compared to mice who were fed a normal diet.

FIGS. 12A-12B. Show the levels of change from baseline in ALT (FIG. 12A) and AST (FIG. 12B) by visit over 6 months of treatment with MSDC-0602K (62.5 mg, 125 mg, 250 mg).

FIGS. 13A-13E: FIG. 13A shows a protocol of C57 mice that were infected with influenza virus A/PR/8/34 (H1N1; ˜200 pfu/mouse) on day 0 through nasal administration of MSDC-0602K and daily body weight; FIG. 13B shows MCP-1 (CCL2) production in the lungs (upper panel) and inflammatory monocyte numbers in the lungs (lower panel); FIG. 13C shows representative histology with a calculation of the percent disrupted area is shown; FIG. 13D shows the expression of alveolar type II cell markers surfactant protein-B (sftbp) and ABCA3 protein (abca3) in the lungs; and FIG. 13E shows total protein levels in bronchoalveolar lavage (BAL, reflective of lung barrier leakage).

FIGS. 14A and 14B show mechanisms of increased ectopic fat in liver and/or pancreas in subjects with COVID-19 infection.

### DETAILED DESCRIPTION

Provided herein are, for example, compounds and compositions that modulate the MPC and have reduced binding and activation of the nuclear transcription factor PPARy. Also provided herein are, for example, methods of treating or preventing a metabolic disorder and/or a respiratory disorder.

MSDC-0602K is a new generation insulin sensitizer that is being developed for the treatment of metabolic disorders such as type 2 diabetes. Unlike first generation thiazolidinedione (TZD) insulin sensitizers which were PPARγ activators, this compound was developed to be more selective for the mitochondrial target of the TZDs, the mitochondrial pyruvate carrier (MPC). By slowing the entry of pyruvate into the mitochondria of all cells, MPC modulators rewire metabolism to improve insulin sensitivity and improve glycemic control in subjects with metabolic disorders such as type 2 diabetes. The pharmacological effect of TZDs and MPC modulators can also include a decrease in inflammation secondary to effects on multiple cell types including macrophages. MSDC-0602K minimizes direct agonism of PPARy, thereby reducing adverse effects caused by direct agonism of PPARy. Thus, MSDC-0602K demonstrates the beneficial effects observed with first generation TZDs, but without the adverse effects that limit the use of first generation TZDs.

Key disturbances in metabolism caused by overnutrition and metabolic dysfunction are mediated by the mitochondrial pyruvate carrier (MPC), which regulates the rate of mitochondrial pyruvate metabolism. In the setting of overnutrition, an excess of pyruvate, an energy source for cells, is rapidly transported into the mitochondria through the MPC, leading to the modification of multiple downstream pathways including transcription factors such as peroxisome proliferator-activated receptors, or PPARs. Downstream effects of this include insulin resistance, increased fat storage, decreased fat oxidation, inflammation, cell damage and fibrosis.

The MPC is a recently discovered protein complex in the inner mitochondrial membrane that mediates the rate of entry of pyruvate—an end product of carbohydrate metabolism and an important source of energy for the cell—into the mitochondria where subsequent oxidative metabolism occurs. This complex is present in the mitochondria of every cell in the body and orchestrates downstream signals that coordinate the cellular machinery, enzymatic pathways and gene expression with the nutritional state and energy need. In animal studies, liver-specific knockout of the MPC has been shown to protect against liver damage, particularly fibrosis, otherwise caused by overnutrition or metabolic dysfunction.

Host responses to respiratory disorders such as coronavirus disease 2019 (COVID-19) differ markedly in subjects depending on age and pre-existing conditions. As with other acute respiratory infections, persons with type 2 diabetes who have been infected with COVID-19 have dire outcomes. This may be because of immune senescence and/or exaggerated inflammatory responses which are a consequence of insulin resistance. Pioglitazone, the first generation TZD insulin sensitizer that is still in use to treat diabetes is contraindicated in many subjects, including those with heart failure or acute respiratory infections because of direct PPARγ effects on fluid balance. A recently completed one-year Phase 2b dose-ranging clinical trial in subjects with fatty liver disease with or without type 2 diabetes demonstrated that treatment with MSDC-0602K could reduce insulin resistance and improve glycemic control without precipitation of edema, the side effect that has limited the use of pioglitazone. Thus, in some embodiments, MSDC-0602K is useful for improving glycemic control or improving metabolic disorders such as diabetes, prediabetes, fatty liver, or insulin resistance syndrome, in subjects with COVID-19 or other respiratory disorders, and may reduce the host response to the infection in a way that will limit the need for hospitalization or other adverse effects. Such an effect may improve outcomes such as respiratory or metabolic outcomes during the resolution of the infection.

Subjects with metabolic disorders such as diabetes or insulin resistance syndrome including fatty liver tend to have more severe adverse respiratory outcomes when they have a respiratory infection such as COVID-19. This may be due to an exaggerated inflammatory response that corresponds with the metabolic disorder. In some embodiments, administration of a composition described herein alleviates metabolic dysfunction or symptoms of the metabolic disorder, and/or reduces symptoms or progression of the respiratory infection by, for example, reducing the exaggerated inflammatory response.

Provided herein, in some embodiments, are methods of treating a metabolic disorder and/or a respiratory disorder by administering a compound that inhibits the MPC in a subject. In some embodiments, the compound does not directly bind PPARγ, or has reduced PPARγ binding compared to a direct PPARγ agonist such as pioglitazone. Some embodiments include treatment of a metabolic disorder. Some embodiments include treatment of a respiratory disorder. Some embodiments include treatment of a respiratory disorder and a metabolic disorder.

Aspects disclosed herein provide compounds of structural Formula (I), or a pharmaceutically acceptable salt thereof, modulates the activity of the MPC can thus exert pleiotropic pharmacology in the context of metabolic dysfunction. In some embodiments, MSDC-0602K modulates the activity of the MPC and can exert pleiotropic pharmacology in the context of context of metabolic dysfunction. Disclosed herein, in some embodiments, are methods of treating a metabolic disorder and/or a respiratory disorder, the treatment comprising administering a compound of structural Formula (I), or a pharmaceutically acceptable salt thereof. In some embodiments, the compound of structural Formula (I), or a pharmaceutically acceptable salt thereof, is MSDC-0602K. In some embodiments, MSDC-0602K is administered as a monotherapy.

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono-, or polyunsaturated and includes mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phosphor, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphatic amino, cycloaliphatic amino, or heterocycloaliphaticamino], sulfonyl [e.g., aliphatic-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-S02-amino)alkyl), aminoalkyl, amidoalkyl, (cycloaliphatic)alkyl, or haloalkyl. An alkoxy is an alkyl attached to the remainder of the molecule via an oxygen linker (—O—).

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to allyl, isoprenyl, 2-butenyl, and 2-hexenyl. An alkenyl group can be optionally substituted with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or cycloaliphaticsulfanyl], sulfinyl [e.g., aliphaticsulfinyl or cycloaliphaticsulfinyl], sulfonyl [e.g., aliphatic-SO₂—, aliphaticamino-SO₂—, or cycloaliphatic-SO₂—], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

As used herein, an “amido” encompasses both “aminocarbonyl” and “carbonylamino”. These terms when used alone or in connection with another group refer to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} are defined below. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—. R^{X} has the same meaning as defined above.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X}, when used as a terminal group; or —OC(O)— or —C(O)O— when used as an internal group.

As used herein, a “haloaliphatic” group refers to an aliphatic group substituted with 1-3 halogens. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂— or the like.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[COQ]ᵥ- where Q is independently a hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, and R₃, and other variables contained in formulae described herein encompass specific groups, such as alkyl and aryl. Unless otherwise noted, each of the specific groups for the variables R₁, R₂, and R₃, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this disclosure are those combinations that result in the formation of stable or chemically feasible compounds.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring.

The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

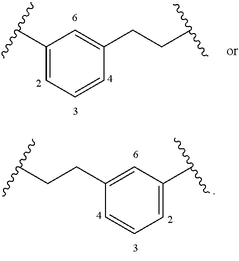 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′, and R″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′, and R″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R″, —ONR′R″, —NR′C(O)NR″NR′″R″″, —CN, —NO₂, —R′, —N₃, —CH(Ph)₂, fluoro(C₁-C₄)alkoxy, and fluoro(C1-C₄)alkyl, —NR'SO₂R″, —NR′C(O)R″, —NR′C(O)—OR″, —NR′OR″, in a number ranging from zero to the total number of open valences on the aromatic ring system; and where R′, R″, R″, and R″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X is —O—, —NW—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.
      (c) A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Some embodiments include a detectable moiety attaA “detectable moiety” as used herein refers to a moiety that can be covalently or noncovalently attached to a compound or biomolecule that can be detected for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dies, radio-labeled moieties, magnetic contrast agents, and quantum dots. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of a compound to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

An “MPC modulator” refers to a compound (e.g., compounds described herein) that directly or indirectly modulate the activity of the MPC when compared to a control, such as absence of the compound or a compound with known inactivity.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. In embodiments, the terms “polypeptide,” “peptide,” and “protein,” used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences, with or without N-terminus methionine residues; immunologically tagged proteins; and the like.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway (e.g., MAP kinase pathway).

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc., refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, an agonist is a molecule that interacts with a target to cause or promote an increase in the activation of the target. In embodiments, activators are molecules that increase, activate, facilitate, enhance activation, sensitize, or up-regulate, e.g., a gene, protein, ligand, receptor, or cell.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease.

As used herein “metabolic inflammation-mediated disease or disorder” refers to disease states where metabolic inflammation is the basis of the pathology. Metabolic inflammation-mediated disease or disorder are diseases or disorders resulting from metabolic inflammation, including but not limited to hypertension, diabetes (e.g., diabetes mellitus type II), diabetes, metabolic syndrome, all aspects of insulin resistance associated with metabolic syndrome (including dyslipidemia and central obesity as well as fatty liver disease and non-alcoholic steatohepatitis (NASH)). In some embodiments, the metabolic inflammation-mediated disease or disorder comprises an “inflammatory disease” or and “autoimmune disease,” as described herein.

As used herein “metabolic syndrome” is a clustering of at least three of the five following medical conditions: abdominal obesity, high blood pressure, high blood sugar, high serum triglycerides and low high-density lipoprotein (HDL) levels and insulin resistance.

As used herein “non-alcoholic fatty liver disease” and “NAFLD” are interchangeable and refer to fatty liver, which occurs when fat is deposited (steatosis) in the liver due to causes other than excessive alcohol use. Non-alcoholic fatty liver disease (NAFLD) may be related to insulin resistance and the metabolic syndrome.

As used herein “non-alcoholic steatohepatitis” and “NASH” are interchangeable and refers to the a form of non-alcoholic fatty liver disease (NAFLD) as defined by histopathology, particularly hepatocyte ballooning and fibrotic scarring. Exemplary diseases and phenotypes associated with NASH include but are not limited to fibrosis, cirrhosis, hepatocellular carcinoma (HCC), liver failure, the need for a liver transplant, portal hypertension, esophageal varices in between cirrhosis and HC, heart failure, myocardial infarcts, coronary and peripheral vascular disease and stroke.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis. Such conditions are frequently inextricably intertwined with other diseases, disorders and conditions. A non-limiting list of inflammatory-related diseases, disorders and conditions which may, for example, be caused by inflammatory cytokines, include, arthritis, kidney failure, lupus, asthma, psoriasis, colitis, pancreatitis, allergies, fibrosis, surgical complications (e.g., where inflammatory cytokines prevent healing), anemia, and fibromyalgia. Other diseases and disorders which may be associated with chronic inflammation include Alzheimer's disease, congestive heart failure, stroke, aortic valve stenosis, arteriosclerosis, osteoporosis, Parkinson's disease, infections, inflammatory bowel disease (IBD), allergic contact dermatitis and other eczemas, systemic sclerosis, transplantation and multiple sclerosis. Some of the aforementioned diseases, disorders and conditions for which a compound (e.g., a MPC modulator) of the present disclosure may be particularly efficacious (due to, for example, limitations of current therapies) are described in more detail hereafter.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal osteomyelitis (CRMO), Churg-Strauss syndrome, Cicatricial pemphigoid/benign mucosal pemphigoid, Crohn's disease, Cogan's syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis Optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

The term “prediabetes,” refers to a condition characterized by a susceptibility to develop diabetes in a subject. In some instances, a subject is “prediabetic” provided an increase in biomarkers indicating a presence or severity of hyperglycemia are detected in a sample obtained from the subject, as compared to a reference level. In some instances, the biomarker comprises bemoglobin A1c (HbA1c). In some cases, the prediabetes refers to pre-type II diabetes.

The terms “treating,” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than occurs absent treatment. In embodiments, prevent refers to slowing the progression of the disease, disorder or condition or inhibiting progression thereof to a harmful or otherwise undesired state.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function enhancing amount,” as used herein, refers to the amount of agonist required to enhance the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of a MPC modulator (or, e.g., a metabolite thereof) at a particular time post-administration may be indicative of whether a therapeutically effective amount has been administered.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent, chemotherapeutic, or treatment for a neurodegenerative disease). The compound of the disclosure can be administered alone or can be co-administered to the patient.

Coadministration is meant to include simultaneous or sequential administration of the compound individually or in combination (more than one compound or agent). Thus, the preparations can also be combined, when desired, with other active substances (e.g. to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. These patents are incorporated herein by reference for such disclosure. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

By “co-administer” it is meant that a composition described herein is administered at the same time, just prior to, or just after the administration of one or more additional therapies. The compounds of the disclosure can be administered alone or can be coadministered to the patient. Coadministration is meant to include simultaneous or sequential administration of the compounds individually or in combination (more than one compound). The compositions of the present disclosure can be delivered transdermally, by a topical route, or formulated as applicator sticks, solutions, suspensions, emulsions, gels, creams, ointments, pastes, jellies, paints, powders, and aerosols.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds (e.g., MPC modulators) disclosed herein can be administered by any acceptable route, such oral, intra-adiposal, intraarterial, intraarticular, intracranial, intradermal, intralesional, intramuscular, intranasal, intra-ocularal, intrapericardial, intraperitoneal, intrapleural, intra-prostatical, intrarectal, intrathecal, intratracheal, intra-tumoral, intra-umbilical, intravaginal, intravenous, intravesicullar, intravitreal, liposomal, local, mucosal, parenteral, rectal, subconjunctival, subcutaneous, sublingual, topical, transbuccal, transdermal, vaginal, in cremes, in lipid compositions, via a catheter, via a lavage, via continuous infusion, via infusion, via inhalation, via injection, via local delivery, via localized perfusion, bathing target cells directly, or any combination thereof.

The compounds (e.g., MPC modulators) disclosed herein may be administered once daily until study reached endpoint. The immune modulator disclosed herein may be administered at least three times but in some studies four or more times depending on the length of the study and/or the design of the study.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule. In some embodiments, a MPC-associated disease modulator is a compound that reduces the severity of one or more symptoms of a disease associated with MPC and/or PPAR (e.g., metabolic inflammation-mediated disease or disorder (e.g., diabetes mellitus type II), metabolic syndrome, non-alcoholic fatty liver disease (NAFLD), and/or non-alcoholic steatohepatitis (NASH)). A MPC modulator is a compound that increases or decreases the activity or function or level of activity or level of function of MPC. A modulator may act alone, or it may use a cofactor, e.g., a protein, metal ion, or small molecule. Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an activator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. In embodiments, the terms “modulate,” “modulation” and the like refer to the ability of a molecule (e.g., an activator or an inhibitor) to increase or decrease the function or activity of MPC or PPAR, either directly or indirectly, relative to the absence of the molecule.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like.

“Substantially pure” indicates that a component makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition (percentage in a weight per weight basis).

The terms “specifically binds” and “selectively binds”, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least 10-times greater, at least 20-times greater, or at least 100-times greater than the affinity with any other antibody, or binding composition derived therefrom. In embodiments, the antibody will have an affinity that is greater than about 10⁹ liters/mol, as determined by, e.g., Scatchard analysis (Munsen, et al. (1980) Analyt. Biochem. 107:220-239).

The terms “DNA,” “nucleic acid,” “nucleic acid molecule,” “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein, the terms “variants” and “homologs” are used interchangeably to refer to amino acid or nucleic acid sequences that are similar to reference amino acid or nucleic acid sequences, respectively. The term encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Thus, variants and homologs encompass naturally occurring amino acid and nucleic acid sequences encoded thereby and their isoforms, as well as splice variants of a protein or gene. The terms also encompass nucleic acid sequences that vary in one or more bases from a naturally-occurring nucleic acid sequence but still translate into an amino acid sequence that corresponds to the naturally-occurring protein due to degeneracy of the genetic code. Non-naturally-occurring variants and homologs include polypeptides and nucleic acids that comprise a change in amino acid or nucleotide sequence, respectively, where the change in sequence is artificially introduced (e.g., muteins); for example, the change is generated in the laboratory by human intervention (“hand of man”). Therefore, non-naturally occurring variants and homologs may also refer to those that differ from the naturally-occurring sequences by one or more conservative substitutions and/or tags and/or conjugates.

The term “muteins” as used herein refers broadly to mutated recombinant proteins. These proteins usually carry single or multiple amino acid substitutions and are frequently derived from cloned genes that have been subjected to site-directed or random mutagenesis, or from completely synthetic genes.

### II. COMPOUNDS

In an aspect provided herein, is a compound having structural Formula (I):

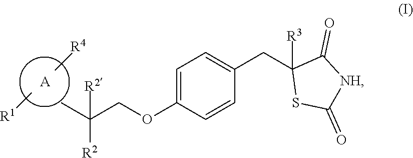

or a pharmaceutically acceptable salt thereof wherein R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R³ is hydrogen.

In other embodiments, R⁴ is independently hydrogen, methyl, or —OR^{4A}; and R^{4A} is independently methyl, ethyl, isopropyl, —CHF₂, or —CF₃. In some embodiments, R⁴ is hydrogen.

In further embodiments, R¹ is hydrogen, halogen or —OR^{1A}; and R^{VA} is substituted or unsubstituted alkyl. In another embodiments R^{VA} is substituted or unsubstituted C₁-C₃alkyl. In other embodiments, R^{1A} is —CHF₂ or —CF₃. In some embodiments, R¹ is hydrogen. In a further embodiment, R¹ is —OR^{1A}; and R^{1A} is substituted or unsubstituted alkyl. In other embodiments, R¹ is halogen. In some embodiments, R¹ is —F or —Cl. In further embodiments, R¹ is attached to the para or meta position of the phenyl. In other embodiments, R¹ is attached to the ortho or meta position of the phenyl. In further embodiments, R¹ is attached to the meta position of the phenyl.

In some embodiments, R^{2′} is hydrogen. In some other embodiments, R² is hydroxyl. In further embodiments, R² and R^{2′} are joined to form oxo.

In some embodiments, the compound of Formula (I) is:

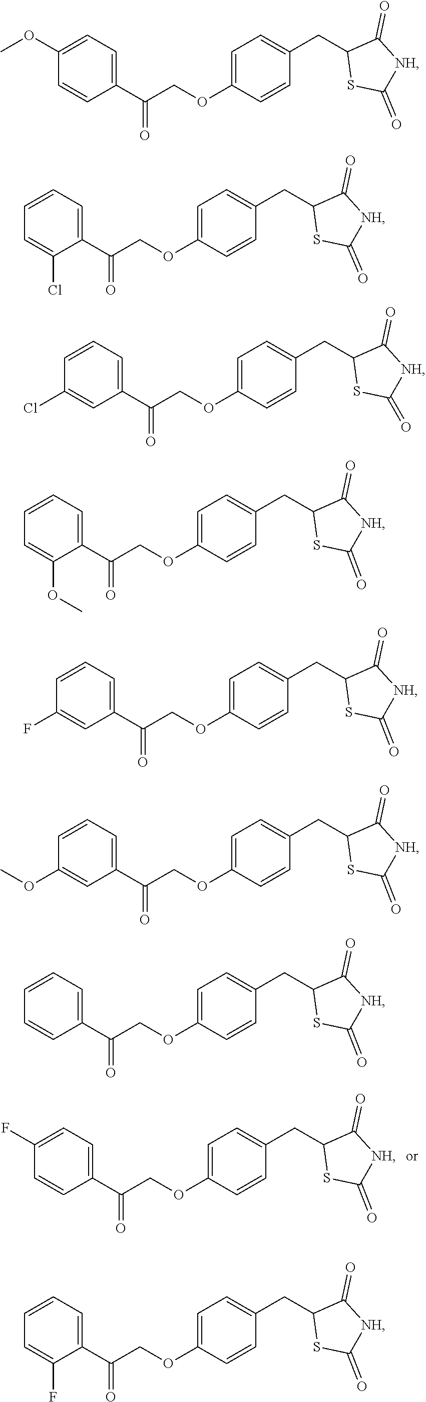

or a pharmaceutically acceptable salt thereof.

In another embodiment, the compound of Formula (I) is:

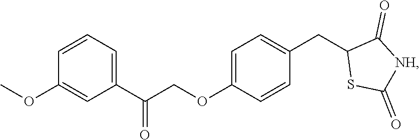 [SMILES: COc1cccc(C(=O)COc2ccc(CC3SC(=O)NC3=O)cc2)c1]

or a pharmaceutically acceptable salt thereof, also termed MSDC-0602. In some embodiments, the potassium salt of

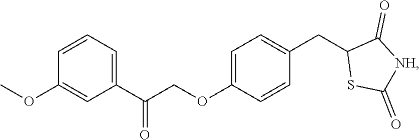 [SMILES: COc1cccc(C(=O)COc2ccc(CC3SC(=O)NC3=O)cc2)c1]

### is MSDC-0602K.

In some embodiments, the compound inhibits a mitochondrial pyruvate carrier (MPC). In some embodiments, the compound has reduced PPAR binding, as compared to one or more direct PPARγ agonists such as pioglitazone.

### III. COMPOSITIONS

In an aspect is provided a composition, including (i) a compound having structural Formula (I):

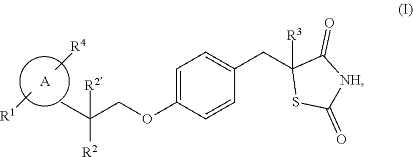

or a pharmaceutically acceptable salt thereof. In the compound having structural Formula (I), R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a pharmaceutical composition, including a compound as described herein, including embodiments, or the structural Formula (I), and at least one pharmaceutically acceptable excipient.

In embodiments, the pharmaceutically acceptable salt is a potassium salt.

The compounds (e.g., MPC or PPAR modulator(s)) of the present disclosure may be in the form of compositions suitable for administration to a subject. In general, such compositions are “pharmaceutical compositions” comprising a compound (e.g., MPC or PPAR modulator(s)) and one or more pharmaceutically acceptable or physiologically acceptable diluents, carriers or excipients. In certain embodiments, the compounds (e.g., MPC or PPAR modulator(s)) are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein.

The pharmaceutical compositions containing the active ingredient (e.g., a modulator of MPC or PPAR function) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture thereof. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a MPC or PPAR modulator contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a MPC or PPAR modulator, including implants (e.g., implantable pumps) and catheter systems, slow injection pumps and devices, all of which are well known to the skilled artisan.

Depot injections, which are generally administered subcutaneously or intramuscularly, may also be utilized to release the compound (e.g., MPC or PPAR modulator) disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, N.J.) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

The present disclosure contemplates the administration of the compound (e.g., MPC or PPAR modulator) in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The compound (e.g., MPC or PPAR modulator) contemplated by the present disclosure may be in the form of any other suitable pharmaceutical composition (e.g., sprays for nasal or inhalation use) currently known or developed in the future.

Some embodiments relate to a composition described herein for use in a subject diagnosed with a respiratory disorder. Some embodiments relate to a composition described herein for use in a subject suspected of having a respiratory disorder. In some embodiments, the composition comprises a compound described herein. In some embodiments, the composition comprises a dose of a compound described herein. In some embodiments, the composition comprises a dosage amount of between about 60 milligrams (mg) and about 250 mg of a compound described herein. For example, the compounds may be MSDC-0602, a compound of structural Formula (I), or a pharmaceutically acceptable salt thereof. Some embodiments relate to a pharmaceutical composition comprising a dosage amount of between about 60 milligrams (mg) and about 250 mg of the compound of structural Formula (I), or a pharmaceutically acceptable salt thereof, for use in a subject diagnosed with a respiratory disorder. In some embodiments, the respiratory disorder comprises a coronavirus infection such as COVID-19. In some embodiments, the subject has or is suspected of having a metabolic disorder such as diabetes, prediabetes, fatty liver, or insulin resistance.

### IV. METHODS

Provided herein are methods that include use of a compound that inhibits a mitochondrial pyruvate carrier (MPC). The compound may be a compound, or be included in a composition described herein. Some embodiments relate to treatment of a disorder. For example, the composition may be administered to a subject for treatment of a disorder. The disorder may include a metabolic disorder and/or a respiratory disorder. Some embodiments include administration of a compound described herein to a subject in need. Some embodiments include administration of a composition described herein to a subject in need. In some embodiments, the subject has a respiratory disorder. In some embodiments, the subject has a metabolic disorder. In some embodiments, the subject has a respiratory disorder and a metabolic disorder. In some cases, the composition is administered when use of a direct PPARγ agonist such as pioglitazone or rosiglitazone would be contraindicated.

Examples of compounds that may be administered according to the methods described herein include a therapeutically effective amount of a compound of structural Formula (I):

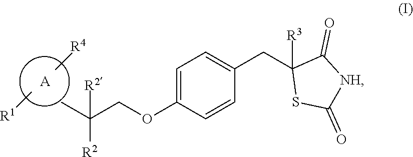

or a pharmaceutically acceptable salt thereof wherein R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R³ is hydrogen.

In other embodiments, R⁴ is independently hydrogen, methyl, or —OR^{4A}; and R^{4A} is independently methyl, ethyl, isopropyl, —CHF₂, or —CF₃. In further embodiments, R⁴ is hydrogen.

In some embodiments, R¹ is hydrogen, halogen or —OR^{1A}; and R^{VA} is substituted or unsubstituted alkyl. In other embodiments, R^{VA} is substituted or unsubstituted C₁-C₃alkyl. In some embodiments, R^{VA} is —CHF₂ or —CF₃. In further embodiments, R¹ is hydrogen. In yet further embodiments, R¹ is —OR^{1A}; and R^{VA} is substituted or unsubstituted alkyl. In some embodiments, R¹ is halogen. In some other embodiments, R¹ is —F or —Cl. In further embodiments, R¹ is attached to the para or meta position of the phenyl; or R¹ is attached to the ortho or meta position of the phenyl. In yet further embodiments, R¹ is attached to the meta position of the phenyl.

In some other embodiments, R^{2′} is hydrogen. In further embodiments, R² is hydroxyl. In some embodiments, R² and R^{2′} are joined to form oxo.

In other embodiments, the compound of Formula (I) is:

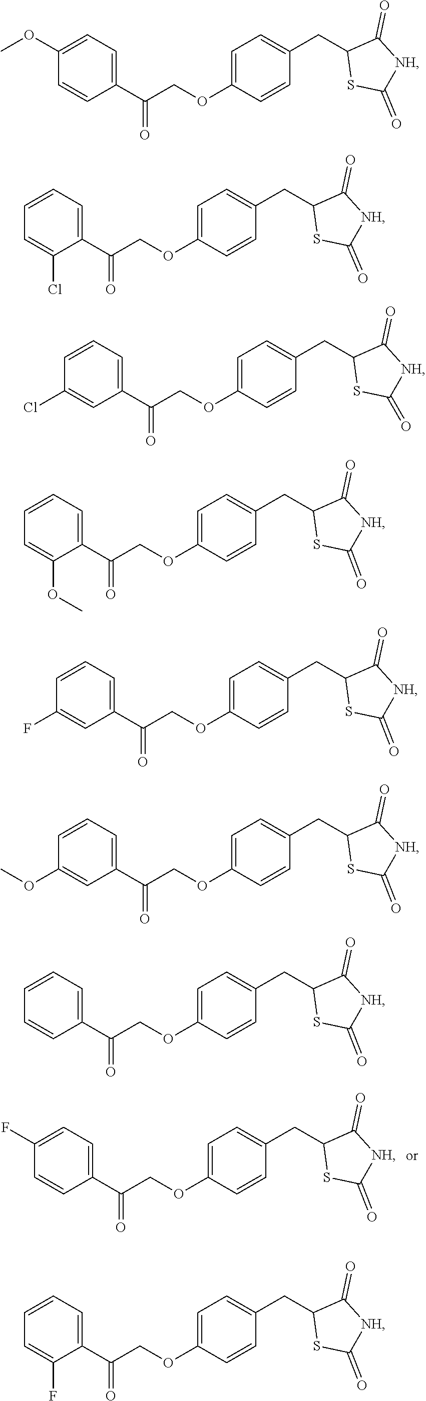

or a pharmaceutically acceptable salt thereof.

In further embodiments, the compound of Formula (I) is:

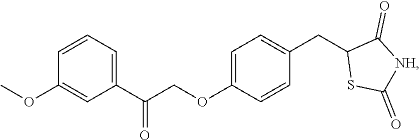 [SMILES: COc1cccc(C(=O)COc2ccc(CC3SC(=O)NC3=O)cc2)c1]

or a pharmaceutically acceptable salt thereof.

In some embodiments, the pharmaceutically acceptable salt is a potassium salt.

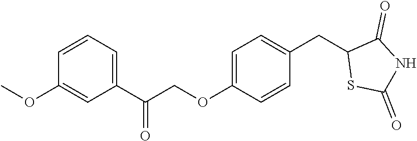 [SMILES: COc1cccc(C(=O)COc2ccc(CC3SC(=O)NC3=O)cc2)c1]

In some embodiments, the potassium salt of is MSDC-0602K. In some embodiments, the composition comprises MSDC-0602K. In some embodiments, the compound comprises MSDC-0602K. In some embodiments, the compound consists of MSDC-0602K.

In another embodiment, MSDC-0602K has the structure:

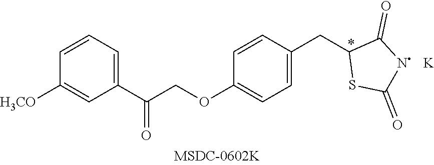

In embodiments, the compound of Formula (I), or a pharmaceutically acceptable salt thereof, is administered orally.

In embodiments, the compound of Formula (I), or a pharmaceutically acceptable salt thereof, is formulated as a tablet or capsule.

In other embodiments is a tablet comprising a compound of Formula (I), or a pharmaceutically acceptable potassium salt thereof; and excipients selected from anhydrous lactose, magnesium stearate, microcrystalline cellulose, sodium croscarmellose, povidone K-30, colloidal silicon dioxide and opadry. In further embodiments, the tablet comprises MSDC-0602K. In yet a further embodiment the tablet comprises about 62.5 mg of MSDC-0602K. In another embodiment, the tablet comprises about 125 mg of MSDC-0602K. In yet another embodiment, the tablet comprises about 250 mg of MSDC-0602K. In another embodiment, treatment may be optimized by initial treatment with 250 mg to achieve an acute benefit followed by chronic treatment at a lower dose.

In some embodiments, the administration of the compound inhibits a mitochondrial pyruvate carrier (MPC) in the subject. In some embodiments, the compound has reduced PPAR binding, as compared to a direct PPARγ agonist. Non-limiting examples of direct PPARγ agonists include pioglitazone or rosiglitazone.

In some embodiments, the subject is suspected of having a metabolic disorder. In some embodiments, the subject has a metabolic disorder. Some embodiments include identifying the subject as having or being suspected of having the metabolic disorder. Some embodiments include identifying the subject as having the metabolic disorder. In some embodiments, the metabolic disorder is a metabolic inflammation-mediated disease or disorder. In some embodiments, the metabolic disorder comprises diabetes. In some embodiments, the metabolic disorder comprises prediabetes. In some embodiments, the diabetes comprises diabetes mellitus type II. In some embodiments, the diabetes does not include type I diabetes. In some embodiments, the metabolic disorder comprises insulin resistance. In some embodiments, the metabolic disorder comprises hyperinsulinemia. In some embodiments, the metabolic disorder comprises glucose intolerance. In some embodiments, the metabolic disorder comprises an increased HOMA-IR relative to a control subject. In some embodiments, the metabolic disorder comprises hyperglycemia. In some embodiments, the metabolic disorder comprises atherosclerotic cardiovascular diseases. In some embodiments, the metabolic disorder comprises a liver disorder such as NASH. In some embodiments, the metabolic disorder comprises a liver disorder such as NAFLD. In some embodiments, the metabolic disorder comprises hyperlipidemia. In some embodiments, the metabolic disorder comprises an increase in a glycated hemoglobin such as HbA1c relative to a control. In some embodiments, the metabolic disorder comprises chronic kidney disease (CKD) stage 1, stage 2, stage 3, stage 4, stage 5, or any combinations thereof. CKD is defined as kidney damage or a decrease in kidney function that persists over three months. The GFR (glomerular filtration rate) is a formula that uses age, race, gender and serum creatinine level to measure kidney function. Patients who have a GFR less than 60 ml/min/1.73 m2 for three months or more are diagnosed as having CKD. Kidney disease progresses as the number of nephrons (filtering units) diminish. The National Kidney Foundation's Kidney Disease Outcomes Quality Initiative for Clinical Practice Guidelines on CKD outlines the following stages for evaluation, classification and treatment strategy: Stage 1—GFR=90 ml/min/1.73 m2 Kidney damage with normal or high GFR; Stage 2— GFR=60 to 89 ml/min/1.73 m2 Kidney damage with mild decreased GFR; Stage 3— GFR=30 to 59 ml/min/1.73 m2 Moderately decreased GFR; Stage 4— GFR=15 to 29 ml/min/1.73 m2 Severely decreased GFR; and Stage 5— GFR=<15 ml/min/1.73 m2 Kidney Failure.

In some embodiments, a baseline measurement of the metabolic disorder is measured or obtained from a baseline sample obtained from the subject. In some cases, the baseline sample is a fluid sample. In some cases, the baseline sample is a blood sample. In some cases, the baseline sample is a plasma sample. In some cases, the baseline sample is a serum sample. In some cases, the baseline sample is a blood, plasma, or serum sample. For example, baseline glucose measurements, baseline insulin measurements, baseline lipid measurements, baseline HOMA-IR measurements, baseline glucose tolerance, or baseline glycated hemoglobin measurements, may be obtained in blood, plasma, or serum samples. In some embodiments, the baseline measurement is a measure of inflammation such as the presence or an amount of one or more inflammatory cytokines. In some cases, the baseline sample is a tissue sample such as a liver sample, a muscle sample, or an adipose sample. In some embodiments, a baseline measurement metabolic disorder is measured directly in or on the subject, or obtained directly from the subject. In some embodiments, multiple baseline measurements are obtained, or the baseline measurement is obtained from multiple samples.

In some embodiments, the subject is suspected of having a respiratory disorder. In some embodiments, the subject has the respiratory disorder. Some embodiments include identifying the subject as having or being suspected of having the respiratory disorder. Some embodiments include identifying the subject as having the respiratory disorder. In some embodiments, the respiratory disorder comprises a respiratory infection. In some embodiments, the respiratory disorder comprises an acute respiratory infection. In some embodiments, the infection is a viral infection. In some embodiments, the respiratory disorder comprises a coronavirus infection. In some embodiments, the subject has a coronavirus infection. In some embodiments, the subject is suspected of having a coronavirus infection. Some embodiments include identifying the subject as having or being suspected of having the coronavirus infection. Some embodiments include identifying the subject as having the coronavirus infection. In some embodiments, the coronavirus comprises severe acute respiratory syndrome coronavirus 2 (SARS-CoV-2). In some embodiments, the coronavirus infection comprises coronavirus disease 2019 (COVID-19). In some embodiments, the coronavirus comprises severe acute respiratory syndrome coronavirus (SARS-CoV). In some embodiments, the coronavirus infection comprises severe acute respiratory syndrome (SARS). In some embodiments, the coronavirus comprises Middle East respiratory syndrome coronavirus (MERS-CoV). In some embodiments, the coronavirus infection comprises Middle East respiratory syndrome (MERS). In some embodiments, the respiratory disorder comprises pneumonia.

In some embodiments, a baseline measurement of the respiratory disorder is measured or obtained from a baseline sample obtained from the subject. In some cases, the baseline sample is a fluid sample. In some cases, the baseline sample is a blood sample. In some cases, the baseline sample is a plasma sample. In some cases, the baseline sample is a serum sample. In some cases, the baseline sample is a blood, plasma, or serum sample. For example, baseline virus particle measurements (such as virus nucleic acids, or virus peptides or glycoproteins) may be obtained in blood, plasma, or serum samples. In some embodiments, the baseline measurement is a measure of inflammation such as the presence or an amount of one or more inflammatory cytokines. In some cases, the baseline sample is a tissue sample such as a lung or airway tissue sample. For example, baseline virus particle measurements (such as virus nucleic acids, or virus peptides or glycoproteins) may be obtained in lung tissue samples or in airway tissue samples. In some cases, the baseline sample is or includes tears, a nasal fluid, saliva, an airway fluid such as a lung fluid. For example, baseline virus particle measurements (such as virus nucleic acids, or virus peptides or glycoproteins) may be obtained in such samples. In some embodiments, a baseline measurement respiratory disorder is measured directly in or on the subject, or obtained directly from the subject. In some cases, the baseline measurement is a clinical parameter. In some cases, the baseline measurement is an oxygen saturation measurement. In some cases, the baseline measurement is a respiratory rate. In some cases, the baseline measurement is a body temperature. In some cases, the baseline measurement is a dyspnea determination. In some cases, the baseline measurement is or includes one or more coughs. In some cases, the baseline measurement is a presence, frequency, duration, or severity of the one or more coughs. In some cases, the baseline measurement is a need for oxygen treatment. In some cases, the baseline measurement is a pneumonia measurement such as the presence or an amount of pneumonia. In some cases, the baseline measurement is a lung fluid measurement. In some embodiments, multiple baseline measurements are obtained, or the baseline measurement is obtained from multiple samples.

In some embodiments, the subject is suspected of having a respiratory disorder, having the respiratory disorder, suspected of having a metabolic disorder, having the metabolic disorder, or any combinations thereof. In some embodiments, the subject is having the respiratory disorder and the metabolic disorder. In some embodiment, the subject is having the respiratory disorder and suspected of having the metabolic disorder. In some embodiments, the suspect is suspect of having the respiratory disorder and the metabolic disorder. In some embodiments, the suspect is suspect of having the respiratory disorder and suspect of having the metabolic disorder.

In some embodiments, the administration treats the metabolic disorder. For example, the administration may prevent, reduce, or stop the incidence or severity of the metabolic disorder or of a symptom of the metabolic disorder. In some embodiments, the administration decreases a biomarker associated with the metabolic disorder. In some embodiments, the administration treats multiple metabolic disorders. In some embodiments, the administration improves one or more metabolic parameters.

In some embodiments, the determination of whether the administration treats the subject is made based on a measurement of a metabolic disorder parameter, for example, a follow-up measurement after the administration following a baseline measurement. In some embodiments, a measurement of the metabolic disorder is measured or obtained from a sample obtained from the subject after treatment, or after the administration. In some cases, the sample is a fluid sample. In some cases, the sample is a blood sample. In some cases, the sample is a plasma sample. In some cases, the sample is a serum sample. In some cases, the sample is a blood, plasma, or serum sample. For example, glucose measurements, insulin measurements, lipid measurements, HOMA-IR measurements, glucose tolerance, or glycated hemoglobin measurements, may be obtained in blood, plasma, or serum samples. In some embodiments, the measurement is a measure of inflammation such as the presence or an amount of one or more inflammatory cytokines. In some cases, the sample is a tissue sample such as a liver sample, a muscle sample, or an adipose sample. In some embodiments, a measurement metabolic disorder is measured directly in or on the subject, or obtained directly from the subject. In some embodiments, multiple measurements are obtained, or the measurement is obtained from multiple samples. In some embodiments, one or more of the measurements are compared to a baseline measurement.

In some embodiments, the administration treats the respiratory disorder. For example, the administration may prevent, reduce, or stop the incidence or severity of the respiratory disorder or of a symptom of the respiratory disorder. In some embodiments, the administration decreases a biomarker associated with the respiratory disorder. In some embodiments, the administration improves one or more respiratory parameters.

In some embodiments, the administration does not have adverse respiratory effects. Non-limiting examples of such adverse respiratory effects include fluid accumulation in a lung of the subject, or pneumonia in the subject. In some embodiments, administration of a direct PPARγ agonist is contraindicated. Non-limiting examples of PPARγ agonists include pioglitazone and rosiglitazone. Some embodiments include determining that administration of the direct PPARγ agonist is contraindicated. In some embodiments, the administration has less adverse respiratory effects than a thiazolidinedione such as pioglitazone. In some embodiments, the contact results in a decreased severity or amount of adverse respiratory outcomes in the subject compared to an administration of a direct PPARγ agonist. In some embodiments, the direct PPARγ agonist is pioglitazone. In some embodiments, the administration does not treat the respiratory disorder, but does not have adverse respiratory effects or has less adverse respiratory effects than pioglitazone. In some embodiments, the administration results in a decreased severity of one or more adverse respiratory outcomes in the subject compared to an administration of pioglitazone. In some embodiments, the administration results in a decreased amount of adverse respiratory outcomes in the subject compared to an administration of pioglitazone. In some embodiments, the administration results in a decreased severity or amount of adverse respiratory outcomes in the subject compared to an administration of pioglitazone.

In some embodiments, the determination of whether the administration treats the subject is made based on a measurement of a respiratory disorder parameter, for example, a follow-up measurement after the administration following a measurement. In some embodiments, a measurement of the respiratory disorder is measured or obtained from a sample obtained from the subject after treatment, or after the administration. In some cases, the sample is a fluid sample. In some cases, the sample is a blood sample. In some cases, the sample is a plasma sample. In some cases, the sample is a serum sample. In some cases, the sample is a blood, plasma, or serum sample. For example, virus particle measurements (such as virus nucleic acids, or virus peptides or glycoproteins) may be obtained in blood, plasma, or serum samples. In some embodiments, the measurement is a measure of inflammation such as the presence or an amount of one or more inflammatory cytokines. In some cases, the sample is a tissue sample such as a lung or airway tissue sample. For example, virus particle measurements (such as virus nucleic acids, or virus peptides or glycoproteins) may be obtained in lung tissue samples or in airway tissue samples. In some cases, the sample is or includes tears, a nasal fluid, saliva, an airway fluid such as a lung fluid. For example, virus particle measurements (such as virus nucleic acids, or virus peptides or glycoproteins) may be obtained in such samples. In some embodiments, a measurement respiratory disorder is measured directly in or on the subject, or obtained directly from the subject. In some cases, the measurement is a clinical parameter. In some cases, the measurement is an oxygen saturation measurement. In some cases, the measurement is a respiratory rate. In some cases, the measurement is a body temperature. In some cases, the measurement is a dyspnea determination. In some cases, the measurement is or includes one or more coughs. In some cases, the measurement is a presence, frequency, duration, or severity of the one or more coughs. In some cases, the measurement is a need for oxygen treatment. In some cases, the measurement is a pneumonia measurement such as the presence or an amount of pneumonia. In some cases, the measurement is a lung fluid measurement. In some embodiments, multiple measurements are obtained, or the measurement is obtained from multiple samples. In some embodiments, one or more of the measurements are compared to a baseline measurement. A determination of whether the composition treats the respiratory disorder may include a measurement of viral infection, a measurement of viral replication, a measurement of mTOR activation, or a measurement of macrophage function. A determination of whether the composition treats the respiratory disorder may include a blood clot measurement such as removal of blood clots, or a number of blood clots.

In some embodiments, administration of a direct PPARγ agonist such as pioglitazone and/or rosiglitazone is contraindicated. For example, pioglitazone may be contraindicated due to edema, or due to fluid accumulation in the lungs. Some embodiments include determining that administration of the direct PPARγ agonist is contraindicated. In some embodiments, the administration of a compound disclosed herein does not result in adverse respiratory side-effects such as fluid accumulation, or exacerbate the respiratory disorder. The contraindication of the direct PPARγ agonist may be due to a likelihood of an adverse respiratory outcome. Examples of adverse respiratory outcomes that may be measured include, but are not limited to, a low oxygen saturation, a low respiratory rate, a fever or increased body temperature, the presence of dyspnea, a presence, frequency, duration, or severity of one or more coughs, and a need for oxygen treatment.

Disclosed herein, in some embodiments, are methods of treatment. Some embodiments include administering a composition described herein to a subject in need thereof, wherein the subject has a metabolic disorder or a coronavirus infection. Some embodiments include administering a composition described herein to a subject in need thereof, wherein the subject has a metabolic disorder and a coronavirus infection. The composition may include, but is not limited to, MSDC-0602K, a compound of structural Formula (I), or a pharmaceutically acceptable salt thereof. Some embodiments include administering to a subject in need thereof a therapeutically effective amount of a compound of structural Formula (I), or a pharmaceutically acceptable salt thereof; wherein the subject has a metabolic disorder and a coronavirus infection. In some embodiments, the coronavirus infection is COVID-19. The metabolic disorder may include, but is not limited to, diabetes, prediabetes, or insulin resistance. In some embodiments, the compound is administered when administration of a direct PPARγ agonist would be contraindicated.

Disclosed herein, in some embodiments, are methods of treating a respiratory disorder, comprising administering to a subject in need thereof, and having or being suspected of having the respiratory disorder, a composition described herein. The composition may include, but is not limited to, MSDC-0602K, a compound of structural Formula (I), or a pharmaceutically acceptable salt thereof. Some embodiments include administering to a subject in need thereof, and having or being suspected of having the respiratory disorder a therapeutically effective amount of a compound of structural Formula (I), or a pharmaceutically acceptable salt thereof. In some embodiments, the respiratory disorder is a coronavirus infection such as COVID-19. In some embodiments, the subject has a metabolic disorder such as diabetes, prediabetes, fatty liver, or insulin resistance syndrome. In some embodiments, the compound is administered when administration of a direct PPARγ agonist would be contraindicated.

Disclosed herein, in some embodiments, are methods of treating a metabolic disorder, comprising administering to a subject in need thereof, and having or being suspected of having the metabolic disorder, a composition described herein. The composition may include, but is not limited to, MSDC-0602K, a compound of structural Formula (I), or a pharmaceutically acceptable salt thereof. Some embodiments include administering to a subject in need thereof, and having or being suspected of having the metabolic disorder, a therapeutically effective amount of a compound of structural Formula (I), or a pharmaceutically acceptable salt thereof. In some embodiments, the metabolic disorder is diabetes, prediabetes, or insulin resistance. In some embodiments, the subject has a respiratory disorder such as a coronavirus infection. In some embodiments, the compound is administered when administration of a direct PPARγ agonist would be contraindicated.

Disclosed herein, in some embodiments, are methods of inhibiting a mitochondrial pyruvate carrier (MPC) in a subject having or suspected of having a respiratory disorder. Some embodiments include contacting the MPC with a compound described herein. In some embodiments, the MPC is a hepatic MPC. The compound may include, but is not limited to, MSDC-0602K, a compound of structural Formula (I), or a pharmaceutically acceptable salt thereof. Some embodiments include inhibiting a mitochondrial pyruvate carrier (MPC) in a subject having or suspected of having a respiratory disorder, comprising contacting the MPC with a compound of structural Formula (I), or a pharmaceutically acceptable salt thereof. In some embodiments, the respiratory disorder is a coronavirus infection such as COVID-19. In some embodiments, the subject has a metabolic disorder such as diabetes, prediabetes, fatty liver, or insulin resistance syndrome. In some embodiments, the MPC is contacted with the compound when administration of a direct PPARγ agonist would be contraindicated.

Disclosed herein, in some embodiments, are methods of increasing glucose tolerance and/or insulin sensitivity. Some embodiments include administering to a subject in need thereof, a therapeutically effective amount of a compound described herein. In some embodiments, the subject has or is suspected of having a respiratory disorder. The compound may include, but is not limited to, MSDC-0602K, a compound of structural Formula (I), or a pharmaceutically acceptable salt thereof. Some embodiments include a method of improving or increasing glucose tolerance and/or insulin sensitivity, comprising administering to a subject in need thereof, a therapeutically effective amount of a compound of structural Formula (I), or a pharmaceutically acceptable salt thereof, wherein the subject has or is suspected of having a respiratory disorder. In some embodiments, the respiratory disorder is a coronavirus infection such as COVID-19. In some embodiments, the subject has a metabolic disorder such as diabetes, prediabetes, fatty liver, or insulin resistance syndrome. In some embodiments, the compound is administered when administration of a direct PPARγ agonist would be contraindicated.

Disclosed herein, in some embodiments, are methods of reducing alanine transaminase (ALT) and/or aspartate aminotransferase (AST) in a subject. In some embodiments, the subject is diagnosed with a respiratory disorder. Some embodiments include administering to the subject a therapeutically effective amount of a compound described herein. The compound may include, but is not limited to, MSDC-0602K, a compound of structural Formula (I), or a pharmaceutically acceptable salt thereof. Some embodiments include a method of reducing alanine transaminase (ALT) and/or aspartate aminotransferase (AST) in a subject diagnosed with a respiratory disorder, the method comprising administering to the subject a therapeutically effective amount of a compound of structural Formula (I), or a pharmaceutically acceptable salt thereof. In some embodiments, the respiratory disorder is a coronavirus infection such as COVID-19. In some embodiments, the subject has a metabolic disorder such as diabetes, prediabetes, fatty liver, or insulin resistance syndrome. In some embodiments, the compound is administered when administration of a direct PPARγ agonist would be contraindicated.

Disclosed herein, in some embodiments, are methods of reducing hemoglobin A1c (HbA1c) in a subject. In some embodiments, the subject is diagnosed with diabetes and/or a respiratory disorder. Some embodiments include administering to the subject a therapeutically effective amount of a compound described herein. The compound may include, but is not limited to, MSDC-0602K, a compound of structural Formula (I), or a pharmaceutically acceptable salt thereof. Some embodiments include a method of reducing hemoglobin A1c (HbA1c) in a subject diagnosed with diabetes and having or suspected of having a respiratory disorder, the method comprising administering to the subject a therapeutically effective amount of a compound of structural Formula (I), or a pharmaceutically acceptable salt thereof. In some embodiments, the respiratory disorder is a coronavirus infection such as COVID-19. In some embodiments, the subject has insulin resistance. The diabetes may be type II diabetes. In some embodiments, the compound is administered when administration of a direct PPARγ agonist would be contraindicated.

Disclosed herein, in some embodiments, are methods of inhibiting cellular mitochondrial pyruvate carrier (MPC) with reduced PPARγ agonism, as compared to pioglitazone, in a subject. In some embodiments, the subject having or suspected of having a respiratory disorder. Some embodiments include administering to the subject a therapeutically effective amount of a compound described herein. The compound may include, but is not limited to, MSDC-0602K, a compound of structural Formula (I), or a pharmaceutically acceptable salt thereof. Some embodiments include a method of inhibiting cellular mitochondrial pyruvate carrier (MPC) with reduced PPARγ agonism, as compared to pioglitazone, in a subject having or suspected of having a respiratory disorder, the method comprising administering to the subject a therapeutically effective amount of a compound of structural Formula (I), or a pharmaceutically acceptable salt thereof. In some embodiments, the respiratory disorder is a coronavirus infection such as COVID-19. In some embodiments, the subject has a metabolic disorder such as diabetes, prediabetes, fatty liver, or insulin resistance syndrome. In some embodiments, the compound is administered when administration of a direct PPARγ agonist would be contraindicated.

Disclosed herein, in some embodiments, are methods of controlling glycemia in a subject without administering insulin to the subject. Some embodiments include administering to the subject a therapeutically effective amount of a compound described herein. In some embodiments, the administration reduces a circulating glucose level in the subject. In some embodiments, the glucose level is determined in a sample from the subject. In some embodiments, the sample is a blood sample. In some embodiments, the sample is a serum sample. In some embodiments, the sample is a plasma sample. In some embodiments, the sample is a blood, serum, or plasma sample. In some embodiments, the circulating glucose level is reduced compared to a control. In some embodiments, the circulating glucose level is reduced compared to a baseline circulating glucose level in the patient. In some embodiments, the circulating glucose level is reduced by at least 2.5%, 5%, 7.5%, 10%, at least 20%, at least 30%, at least 40%, at least 50%, at least 60%, at least 70%, at least 80%, or at least 90%. In some embodiments, the circulating glucose level is reduced by no more than 10%, no more than 20%, no more than 30%, no more than 40%, no more than 50%, no more than 60%, no more than 70%, no more than 80%, or no more than 90%. The compound may include, but is not limited to, MSDC-0602K, a compound of structural Formula (I), or a pharmaceutically acceptable salt thereof. In some embodiments, the subject has a respiratory disorder. In some embodiments, the respiratory disorder is a coronavirus infection such as COVID-19. In some embodiments, the subject has a metabolic disorder such as diabetes, prediabetes, fatty liver, or insulin resistance syndrome. In some embodiments, the compound is administered when administration of a direct PPARγ agonist would be contraindicated.

In one embodiment is a method of treating a patient having a respiratory disorder and diabetes comprising administering to the patient about 125 mg of MSDC-0602K wherein a biomarker of the respiratory disorder of the patient prior to treatment are elevated compared to a patient having a normal range of the biomarker. In another embodiment, is a method for treating a patient having a respiratory disorder and diabetes comprising administering to the patient about 250 mg of MSDC-0602K wherein the administration results in significant reduction in HOMA-IR, HbA1c, and/or the biomarker. In a further embodiment is a method for treating a patient having a respiratory disorder and diabetes comprising administering to the patient about 125 mg of MSDC-0602K wherein the administration results in significant reduction in HOMA-IR, HbA1c, and/or the biomarker. In another embodiment is a method for treating a patient having a respiratory disorder and diabetes comprising administering to the patient about 62.5 mg of MSDC-0602K. In another embodiment is a method for treating a patient having a respiratory disorder and diabetes comprising administering to the patient about 125 mg of MSDC-0602K. In yet another embodiment is a method for treating a patient having a respiratory disorder and diabetes comprising administering to the patient about 250 mg of MSDC-0602K. In another embodiment, treatment may be optimized by initial treatment with 250 mg to achieve an acute benefit followed by chronic treatment at a lower dose.

In yet a further embodiment is a method of treating a patient having a respiratory disorder and diabetes, prediabetes, fatty liver, or insulin resistance syndrome comprising administering to the patient about 62.5 mg, 125 mg, or 250 mg of MSDC-0602K once a day wherein the patient shows at least one of the following beneficial responses: decreased respiratory disorder biomarkers, improved respiratory function, improved glycemic control, improved outcomes such as cardiovascular, mortality, liver outcomes, and long-term outcomes.) In yet another embodiment, the amount administered to the patient may be varied for example, starting with higher dose such as 250 mg and then after a successful response the dose may be lowered to a dose such as 125 mg. In yet a further embodiment, the patient is confirmed as having the respiratory disorder. In yet another embodiment, the diabetes is Type II diabetes. In another embodiment, the patient has met the ADA criteria for diabetes. In a further embodiment, the patient has been diagnosed by a physician as having diabetes. In another embodiment, the patient has met the classification and diagnosis of diabetes as described in Diabetes Care 2018; 41 (Suppl. 1):S13-S27.

In yet another embodiment, the patient has symptoms similar to a coronavirus infection such as COVID-19 that leads one to believe they may have the coronavirus infection.

In one embodiment is a method for improving glycemic control in patients with Type II diabetes and a coronavirus infection comprising administering to the patient about 125 mg of MSDC-0602K.

In one embodiment, the patient is administered MSDC-0602K in the morning. In another embodiment, the patient is administered MSDC-0602K in the evening. In a further embodiment, the patient is administered MSDC-0602K at night.

In one embodiment is a method of treating a patient having a coronavirus infection and diabetes comprising administering to the patient about 62.5 mg of MSDC-0602K wherein a coronavirus biomarker of the patient prior to treatment is elevated compared to a patient without the coronavirus infection. In another embodiment, is a method for treating a patient having a coronavirus infection and diabetes comprising administering to the patient about 62.5 mg of MSDC-0602K wherein the administration results in significant reduction in HOMA-IR, HbA1c, and/or a coronavirus biomarker.

In one embodiment is a method of treating patients having a coronavirus infection and diabetes comprising: measuring a coronavirus biomarker level in a sample from a patient prior to initial treatment; treating the patient with a compound of Formula (I); measuring the coronavirus biomarker level following the initial treatment; and determining whether treatment with a compound of Formula (I) should continue based on the coronavirus biomarker level as compared to a coronavirus biomarker level in a standard or control sample. In some embodiments, the method comprises assessing patients using the modified WHO COVID-19 ordinal scale (range 0 to 8, with higher ranks representing higher severity) prior to and/or subsequent to treating the patients with a compound of Formula (I) with various dosages and/or using various administration methods. In some embodiments, a 7-point scale (1. Death 2. Hospitalized, on invasive mechanical ventilation or Extracorporeal Membrane Oxygenation 3. Hospitalized, on non-invasive ventilation or high flow oxygen 4. Hospitalized, requiring low flow supplemental oxygen 5. Hospitalized, not requiring supplemental oxygen-requiring ongoing medical care (COVID-19 related or otherwise) 6. Hospitalized, not requiring supplemental oxygen—no longer required ongoing medical care 7. Not hospitalized) may be utilized. Further, viral clearance and/or clinical remission or stability may be used to assess the severity level of a patient's COVID-19 infection.

In one embodiment is a method of treating a prediabetic patient with a coronavirus infection, comprising administering to the patient a therapeutically effective amount of MSDC-0602K. In another embodiment, the therapeutically effective amount is about 62.5 mg, about 125 mg, or about 250 mg.

In one embodiment is a method of treating a patient having insulin resistance with a coronavirus infection, comprising administering to the patient a therapeutically effective amount of MSDC-0602K. In another embodiment, the therapeutically effective amount is about 62.5 mg, about 125 mg, or about 250 mg.

In some embodiments, the present disclosure describes methods for inhibiting a mitochondrial pyruvate carrier (MPC) in a subject having a respiratory disorder in order to treat, prevent, mitigate, or alleviate symptoms associated with medium or long-term outcomes/sequelae of the respiratory disorder. Some embodiments include contacting the MPC with a compound described herein. The compound may include, but is not limited to, MSDC-0602K, a compound of structural Formula (I), or a pharmaceutically acceptable salt thereof. In some embodiments, the respiratory disorder is a coronavirus infection such as COVID-19 or the body's response to the viral infection. In some embodiments, medium-term outcomes of COVID-19 comprise burden and impairment to the vascular system and major organs, such as heart (i.e., myocarditis and systolic dysfunction), lung, liver and pancreas. Further, in some embodiments, long-term outcomes of COVID-19 comprise burden and impairment to major organs, such as heart (i.e., myocarditis and systolic dysfunction), lung, liver and pancreas. The long-term effect of major organ impairment may lead to fatigue, shortness of breath, myalgia, headache and arthralgia and a state of confusion known as “brain fog”. The long-term outcomes of COVID-19 infection may be monitored at least 1 month, 2 months, 3 months, 4 months, 5 months, 6 month, 7 months, 8 months, 9 months, 10 months, 11 months, 12, months, 24 months, 36 months, or 48 months post-COVID-19. In some embodiments, MSDC-0602K or any other suitable MPC inhibitor is administered after at least about 1 month, 2 months, 3 months, 4 months, 5 months, 6 month, 7 months, 8 months, 9 months, 10 months, 11 months, 12, months, 24 months, 36 months, or 48 months post COVID-19 infection. In some embodiments, MSDC-0602K or any other suitable MPC inhibitor is administered after at least about 3 months post COVID-19 infection for about 1 week, 2 weeks, 3 weeks, 4 weeks, 2 months, 3 months, 4 months, 5 months, 6 months, 7 months, 8 months, 9 months, 10 months, 11 months, 12 months, 24 months, 36 months, or 48 months. In some embodiments, MSDC-0602K or any other suitable MPC inhibitor is administered after at least about 4 months post COVID-19 infection for about 1 week, 2 weeks, 3 weeks, 4 weeks, 2 months, 3 months, 4 months, 5 months, 6 months, 7 months, 8 months, 9 months, 10 months, 11 months, 12 months, 24 months, 36 months, or 48 months. In some embodiments, MSDC-0602K or any other suitable MPC inhibitor is administered after at least about 5 months post COVID-19 infection for about 1 week, 2 weeks, 3 weeks, 4 weeks, 2 months, 3 months, 4 months, 5 months, 6 months, 7 months, 8 months, 9 months, 10 months, 11 months, 12 months, 24 months, 36 months, or 48 months. In some embodiments, MSDC-0602K or any other suitable MPC inhibitor is administered after at least about 6 months post COVID-19 infection for about 1 week, 2 weeks, 3 weeks, 4 weeks, 2 months, 3 months, 4 months, 5 months, 6 months, 7 months, 8 months, 9 months, 10 months, 11 months, 12 months, 24 months, 36 months, or 48 months.

In some embodiments, a higher dose of a MPC inhibitor maybe administered first to a subject for a period of time until the subject having, suspected of having, or already had a respiratory disorder, for example, COVID-19, has demonstrated adequate response to the MPC inhibitor in terms of increased adiponectin production, decreased cytokines production, decreased inflammatory response, decreased visceral fat content (e.g., ectopic fat around liver and kidney), or any combinations thereof. Subsequently, a smaller dosage of the MPC inhibitor may be administered to the subject for a second period of time in order to minimize weight gain while maintaining the beneficial effects including reduced ectopic fat content. In some embodiments, the MPC inhibitor is MSDC-0602K. In some embodiments, the first dosage is higher than the second dosage. In some embodiments, the first dosage equals the second dosage. In some embodiments, the first dosage is less than the second dosage. In some embodiments, the first dosage is about at least 100, 125, 150, 200, 250, 300, 350, 400, 450, 500 mg of MSDC-0602K. In some embodiments, the first dosage is about 250 mg of MSDC-0602K. In some embodiments, the second dosage is about at most 250, 200, 150, 125, 100, 62.5, 50, 25, 10, 5 mg of MSDC-0602K. In some embodiments, the second dosage is about 125 mg of MSDC-0602K. In some embodiments, the second dosage is about 62.5 mg of MSDC-0602K.

Further, in some embodiments, the first period of time is shorter than the second period of time. In some embodiments, the first period of time equals the second period of time. In some embodiments, the first period of time is about at most 12 months, 11 months, 10 months, 9 months, 8 months, 7 months, 6 months, 5 months, 4 months, 3 months, 2 months, 1 month, 3 weeks, 2 weeks, 1 week, 6 days, 5 days, 4 days, 3 days, 2 days, or 1 day. In some embodiments, the first period is about 1 month. In some embodiments, the first period is about 2 months. In some embodiments, the first period is about 3 months. In some embodiments, the first period is about 4 months. In some embodiments, the first period is about 5 months. In some embodiments, the first period is about 6 months. In some embodiments, the first period is about 7 months. In some embodiments, the first period is about 8 months. In some embodiments, the first period is about 9 months. In some embodiments, the first period is about 10 months. In some embodiments, the first period is about 11 months. In some embodiments, the first period is about 12 months. In some embodiments, the first period of time is no longer than about 6 months. In some embodiments, the first period of time is no longer than about 5 months. In some embodiments, the first period of time is no longer than about 4 months. In some embodiments, the first period of time is no longer than about 3 months. In some embodiments, the first period of time is no longer than about 2 months. In some embodiments, the first period of time is no longer than about 1 months. In some embodiments, the second period of time is about at least 3 months, 4 months, 5 months, 6 months, 7 months, 8 months, 9 months, 10 months, 11 months, 12 months, 24 months, 36 months, 48 months, 72 months, 7 years, 8 years, 9 years, 10 years, 11 years, 12 years, 15 years, 20 years, 25 years, 30 years, 35 years, 40 years, 45 years, 50 years, 55 years, 60 years, 80 years, or 100 years.

In some embodiments, after administering the first dosage for the first period of time, the subject's hepatic fat content is reduced by at least 5%, 10%, 15%, 20%, 25%, 30%, 35%, 40%, 45%, 50%, 55%, 60%, 65%, 70%, 75%, 80%, 85%, 90%, or 95% from a baseline measurement. The baseline measurement of the hepatic fat content is obtained before administrating the first dosage. Further, in some embodiments, after administering the first dosage for the first period of time, the subject's ectopic fat content in the liver is reduced by at least 5%, 10%, 15%, 20%, 25%, 30%, 35%, 40%, 45%, 50%, 55%, 60%, 65%, 70%, 75%, 80%, 85%, 90%, or 95% from a baseline measurement. The baseline measurement of the ectopic fat content is obtained before administrating the first dosage. In some embodiments, after administering the first dosage for the first period of time, the subject's ectopic fat content in the pancreas is reduced by at least 5%, 10%, 15%, 20%, 25%, 30%, 35%, 40%, 45%, 50%, 55%, 60%, 65%, 70%, 75%, 80%, 85%, 90%, or 95%. In some embodiments, the hepatic fat content or the ectopic fat in the liver and pancreas is measured by a MRI scan or any other suitable scan or methods. In some embodiments the magnitude of the second, maintenance doses of the inhibitor may be adjusted back up to allow for the optimal control of other metabolic parameters such as plasma glucose or circulating liver enzymes.

In certain embodiments, in the treatment, prevention, or amelioration of one or more symptoms of the disorders, diseases, or conditions described herein (e.g., treatment or prevention of at least one metabolic inflammation-mediated disease or disorder, and/or treatment or prevention of at least one respiratory disorder), an appropriate dosage level of a compound of Formula (I), or a pharmaceutically acceptable salt, solvate, hydrate, or prodrug thereof generally is ranging from about 1 to 3000 mg, from about 1 to 2000 mg, from about 1 to 1000 mg, from about 1 to about 500 mg, from about 5 to about 500 mg, from about 5 to about 400 mg, from about 5 to about 300 mg, from about 5 to about 250 mg, from about 5 to about 125 mg or from about 62.5 to about 250 mg, which can be administered in single or multiple doses. In certain embodiments, in the treatment, prevention, or amelioration of one or more symptoms of the disorders, diseases, or conditions described herein (e.g., treatment or prevention of at least one metabolic inflammation-mediated disease or disorder), an appropriate dosage level of a compound of Formula (I), or a pharmaceutically acceptable salt, solvate, hydrate, or prodrug thereof is 62.5 mg, 125 mg, or 250 mg. In certain embodiments, the compound of Formula (I), or a pharmaceutically acceptable salt, solvate, hydrate, or prodrug thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 300, 350, 400, or 500 mg. In certain embodiments, the compound of Formula (I), or a pharmaceutically acceptable salt, solvate, hydrate, or prodrug thereof is administered in an amount of about 60, 60.5, 61, 61.5, 62, 62.5, 63, 63.5, 64, 64.5, 65, 65.5, 66, 66.5, 67, 67.5, 68, 68.5, 69, 69.5, or 70 mg. In certain embodiments, the compound of Formula (I), or a pharmaceutically acceptable salt, solvate, hydrate, or prodrug thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 300, 350, 400, or 500 mg/day. In certain embodiments, the compound of Formula (I), or a pharmaceutically acceptable salt, solvate, hydrate, or prodrug thereof is administered in an amount of about 60, 60.5, 61, 61.5, 62, 62.5, 63, 63.5, 64, 64.5, 65, 65.5, 66, 66.5, 67, 67.5, 68, 68.5, 69, 69.5, or 70 mg/day.

For oral administration, the pharmaceutical compositions provided herein can be formulated in the form of tablets or capsules containing from about 1.0 to about 1,000 mg of a compound of Formula (I), or a pharmaceutically acceptable salt, solvate, hydrate, or prodrug thereof. In certain embodiments, for oral administration, the pharmaceutical compositions provided herein can be formulated in the form of tablets or capsules containing about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 300, about 350, about 400, or about 500 mg of a compound of Formula (I), or a pharmaceutically acceptable salt, solvate, hydrate, or prodrug thereof for the symptomatic adjustment of the dosage to the patient to be treated. In certain embodiments, for oral administration, the pharmaceutical compositions provided herein can be formulated in the form of tablets or capsules containing about 60, 60.5, 61, 61.5, 62, 62.5, 63, 63.5, 64, 64.5, 65, 65.5, 66, 66.5, 67, 67.5, 68, 68.5, 69, 69.5, or 70 mg of a compound of Formula (I), or a pharmaceutically acceptable salt, solvate, hydrate, or prodrug thereof for the symptomatic adjustment of the dosage to the patient to be treated.

The pharmaceutical compositions can be administered on a regimen of one (1) to four (4) times per day, including once, twice, three times, and four times per day. In some embodiments, the compound of Formula (I), or an isotopic variant thereof or a pharmaceutically acceptable salt, solvate, hydrate, or prodrug thereof is administered once per day.

In some embodiments, the compound of Formula (I), or a pharmaceutically acceptable salt thereof, is administered in a dosage of from about 60 mg to about 250 mg. In some embodiments, the compound of Formula (I), or a pharmaceutically acceptable salt thereof, is administered in a dosage of about 62.5 mg. In other embodiments, the compound of Formula (I), or a pharmaceutically acceptable salt thereof, is administered in a dosage of about 125 mg. In further embodiments, the compound of Formula (I), or a pharmaceutically acceptable salt thereof, is administered in a dosage of about 250 mg. In certain embodiments, the compound of Formula (I), or a pharmaceutically acceptable salt thereof, is administered in a dosage of about 125 mg or about 250 mg for a period of time comprising one month, two months, three months, four months, five months, six months, seven months, eight months, nine months, ten months, eleven months, or twelve months. Thereafter, in certain embodiments, the compound of Formula (I), or a pharmaceutically acceptable salt thereof, is administered in a dosage of about 62.5 mg for a period of time comprising one year, two years, three years, four years, five years, six years, seven years, eight years, nine years, ten years, 20 years, 30 years, 40 years, 50 years, 60 years, 70 years, 80 years, or more. In a further embodiment, the compound of Formula (I), or a pharmaceutically acceptable salt thereof is administered in a dosage of about 250 mg for a period of time followed by administration in a dosage of about 125 mg thereafter. In yet a further embodiment, the compound of Formula (I), or a pharmaceutically acceptable salt thereof is administered in a dosage of about 250 mg for a period of time followed by administration in a dosage of about 62.5 mg thereafter. In another embodiment, the compound of Formula (I), or a pharmaceutically acceptable salt thereof is administered in a dosage of about 125 mg for a period of time followed by administration in a dosage of about 62.5 mg thereafter.

In other embodiments, the compound of Formula (I), or a pharmaceutically acceptable salt thereof, is administered daily. In embodiments, the compound of Formula (I), or a pharmaceutically acceptable salt thereof, is administered once daily.

Provided herein is a method of treating a respiratory disorder and/or metabolic syndrome, comprising administering to a subject in need thereof a therapeutically effective amount of a compound as described herein, including embodiments, or the structural Formula (I), or a pharmaceutically acceptable salt thereof.

Provided herein is a method of treating a respiratory disorder and/or at least one metabolic inflammation-mediated disease or disorder, comprising administering to a subject in need thereof a therapeutically effective amount of a pharmaceutical composition comprising:

a compound of structural Formula (I), or a pharmaceutically acceptable salt thereof:

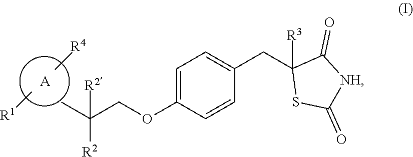

wherein R¹, R², R^{2′}, R³, and R⁴ are as described herein, including embodiments;

and

(iii) and a pharmaceutically acceptable.

In an aspect is provided a method of treating at least one metabolic inflammation-mediated disease or disorder, comprising administering to a subject in need thereof a therapeutically effective amount of a compound of structural Formula (I), or a pharmaceutically acceptable salt thereof:

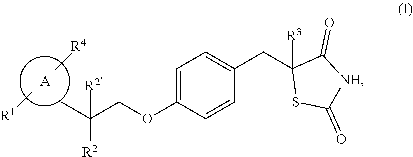

wherein R¹, R², R^{2′}, and R⁴ are as described herein, and R³ is deuterium, wherein the subject has or is suspected of having a respiratory disorder such as a coronavirus infection.

In some embodiments, the subject has the respiratory disorder. In other embodiments, the subject has at least one metabolic inflammation-mediated disease or disorder. In further embodiments, the subject has the respiratory disorder and at least one metabolic inflammation-mediated disease or disorder. In some embodiments, the subject has the respiratory disorder and/or metabolic syndrome and at least one metabolic inflammation-mediated disease or disorder. In other embodiments, the subject has diabetes mellitus, the respiratory disorder, or metabolic syndrome, or any combination thereof. In other embodiments, the subject is suffering from obesity, non-alcoholic fatty liver disease (NAFLD), a metabolic inflammation-mediated disease or disorder, metabolic syndrome, the respiratory disorder, or any combination thereof. In further embodiments, the at least one metabolic inflammation-mediated disease or disorder is diabetes mellitus type II.

In another aspect is provided a method of inhibiting cellular or hepatocyte mitochondrial pyruvate carrier (MPC), comprising contacting the MPC with a compound as described herein, including embodiments, or the structural Formula (I), or a pharmaceutically acceptable salt thereof. In some case, the cell or hepatocyte is from a subject with a respiratory disorder such as a coronavirus infection. In one aspect is provided a method of inhibiting hepatocyte mitochondrial pyruvate carrier (MPC), comprising contacting the MPC with a compound as described herein, including embodiments, or the structural Formula (I). In embodiments, the hepatocyte is in vivo. In embodiments, the hepatocyte is a human hepatocyte.

In an aspect is provided a method of improving or increasing glucose tolerance and/or insulin sensitivity, comprising administering to a subject in need thereof a therapeutically effective amount of a compound as described herein, including embodiments, or the structural Formula (I), or a pharmaceutically acceptable salt thereof. In an aspect is provided a method of improving or increasing glucose tolerance and/or insulin sensitivity, comprising: administering to a subject in need thereof a therapeutically effective amount of a compound as described herein, including embodiments, or the structural Formula (I), or a pharmaceutically acceptable salt thereof. In some cases, the subject has or is suspected of having a respiratory disorder such as a coronavirus infection.

In an aspect provided herein, are methods of reducing a level of a respiratory disorder biomarker in a subject diagnosed with the respiratory biomarker, the method comprising administering to the subject a therapeutically effective amount of a compound of structural Formula (I), or a pharmaceutically acceptable salt thereof. The respiratory disorder may comprise a coronavirus infection. Also provided, in some aspects, are methods of reducing a level of a respiratory disorder biomarker in a subject diagnosed with diabetes, the method comprising administering to the subject a therapeutically effective amount of a compound of structural Formula (I), or a pharmaceutically acceptable salt thereof. In some embodiments the pharmaceutically acceptable salt comprises a potassium salt. Disclosed herein in some embodiments are methods of reducing a level of a respiratory disorder biomarker in a subject who is prediabetic, the method comprising administering to the subject a therapeutically effective amount of a compound of structural Formula (I), or a pharmaceutically acceptable salt thereof. In some embodiments the pharmaceutically acceptable salt comprises a potassium salt.

Also provided, in some aspects, are methods of reducing a level of hemoglobin A1c (HbA1c) in a subject diagnosed with diabetes and having a coronavirus infection, the method comprising administering to the subject a therapeutically effective amount of a compound of structural Formula (I), or a pharmaceutically acceptable salt thereof. In some embodiments the pharmaceutically acceptable salt comprises a potassium salt. Disclosed herein in some embodiments are methods of reducing a level of hemoglobin A1c (HbA1c) in a subject who is prediabetic and has a coronavirus infection, the method comprising administering to the subject a therapeutically effective amount of a compound of structural Formula (I), or a pharmaceutically acceptable salt thereof. In some embodiments the pharmaceutically acceptable salt comprises a potassium salt.

In another aspect provided herein, are methods of inhibiting a mitochondrial pyruvate carrier (MPC) with reduced PPARγ agonism, as compared to pioglitazone, in a subject having or suspected of having a respiratory disorder such as a coronavirus infection, the method comprising administering to the subject a therapeutically effective amount of a compound of structural Formula (I), or a pharmaceutically acceptable salt thereof. In some embodiments the pharmaceutically acceptable salt comprises a potassium salt. In some embodiments, the MPC is a hepatic MPC.

### V. MECHANISM OF ACTION

Insulin sensitizers (e.g., pioglitazone and rosiglitazone) have detrimental side effects due to agonism of the nuclear transcription factor, PPARγ including, edema, weight gain, and bone loss. Agents such as pioglitazone and/or rosiglitazone may, therefore, be contraindicated in some situations, particularly when a subject faces an acute respiratory infection such as COVID-19. Treatment with MSDC-0602K or another compound or composition described herein may reduce the severity of respiratory outcomes during such an infection. Rather than targeting the PPARγ in some such situations, it would be beneficial to target the mitochondrial pyruvate carrier (MPC) for treatment of a patient. In some cases, the MPC in macrophages and/or endothelial cells plays a role in beneficial effects resultant from administering compositions targeting the MPC. Furthermore, MPC inhibitors, e.g., MSDC-0602K, lack detrimental side effects in treating subjects with metabolic disorders and/or respiratory disorders. When treating a subject with metabolic disorders and/or respiratory disorders, pioglitazone and rosiglitazone, cannot be dosed at a high dose initially and has to be dosed at a low dose and only increase as subject tolerates the dose. This dosing regimen may lead to a prolonged treatment period of the subject and may be less effective during the beginning of the treatment due to lower doses.

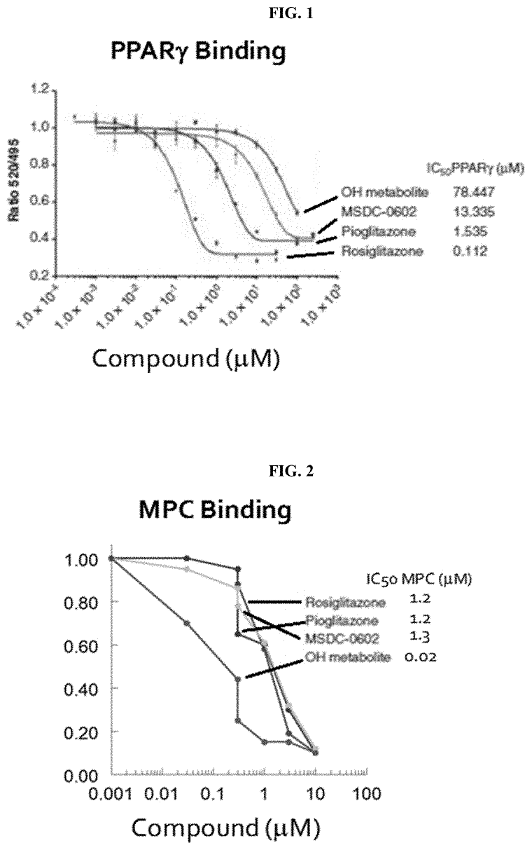

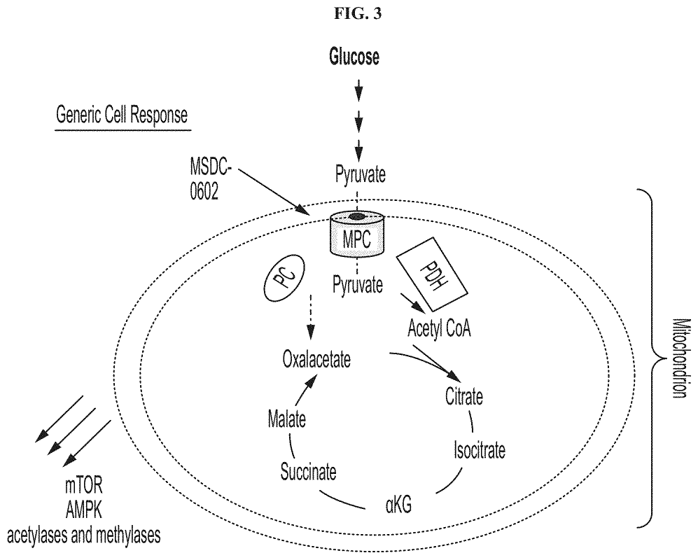

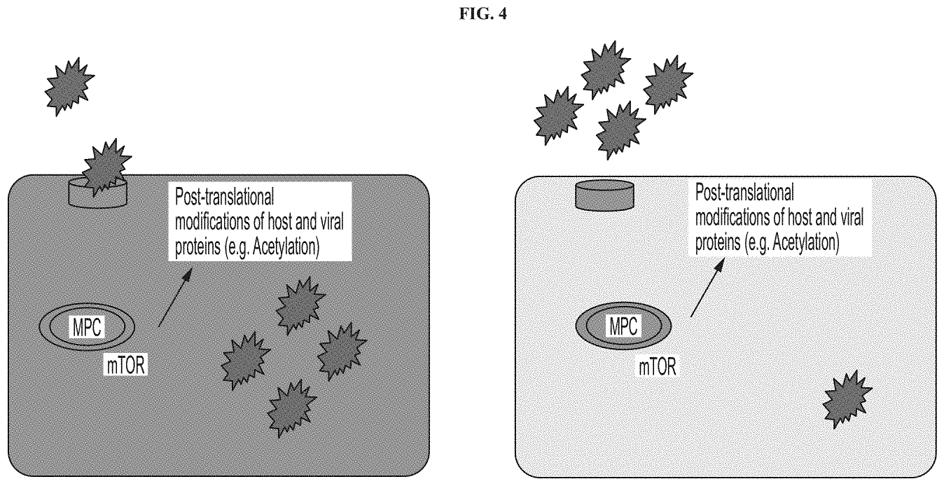

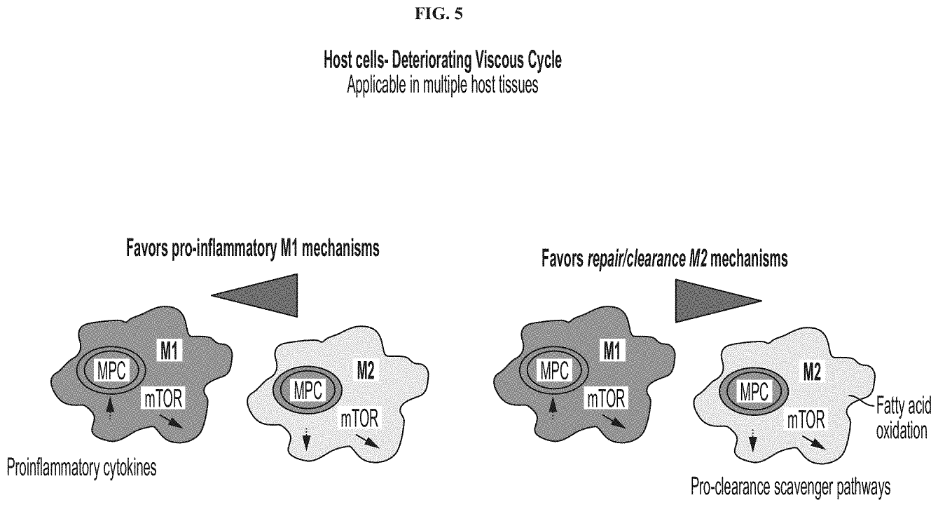

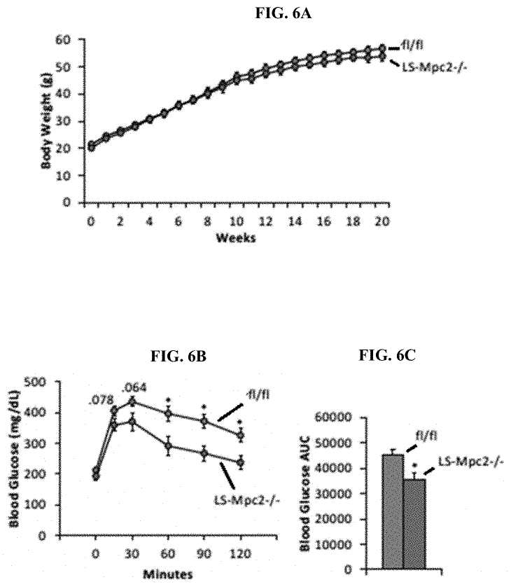

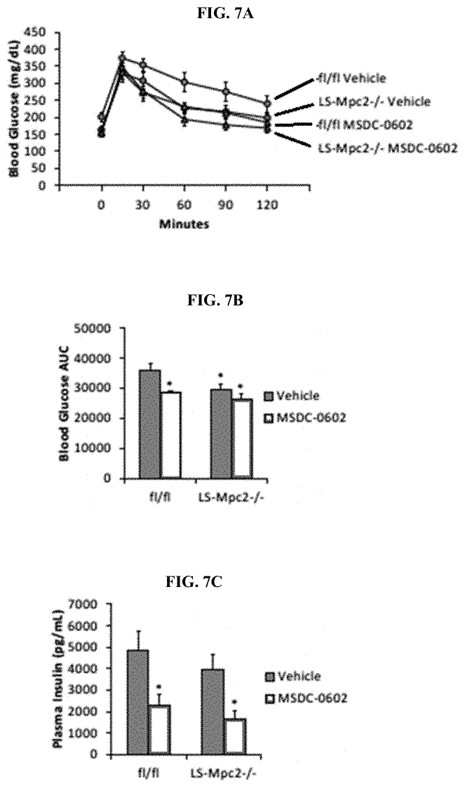

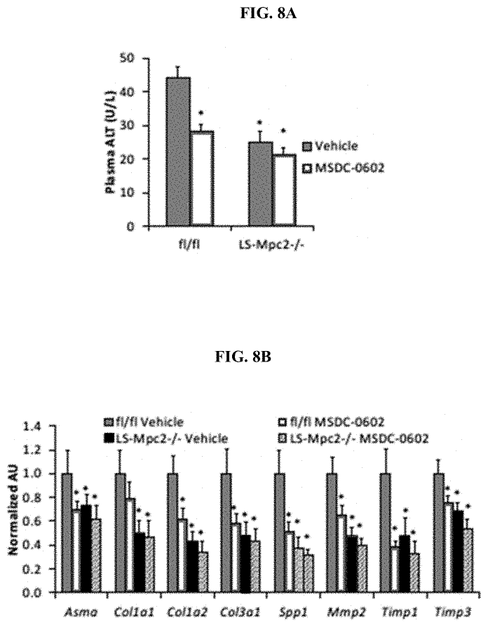

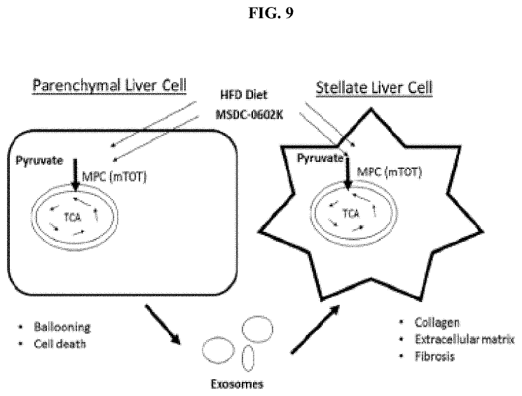

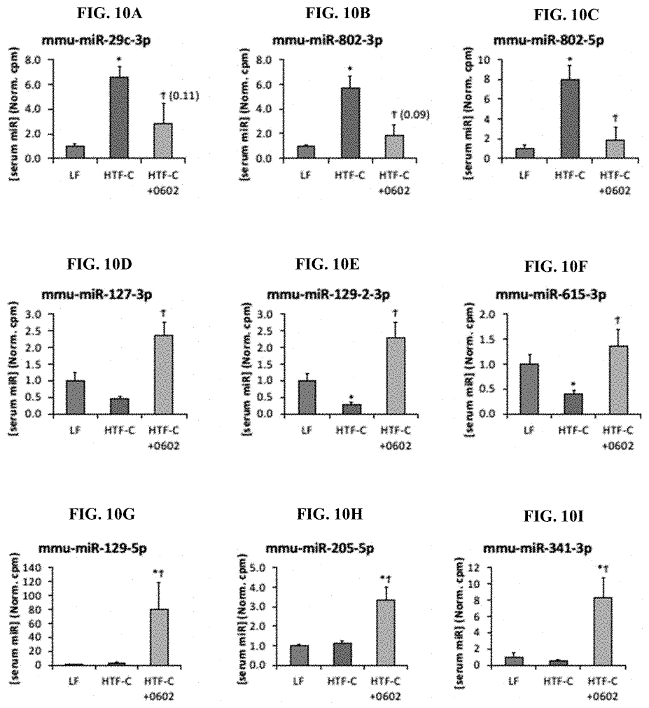

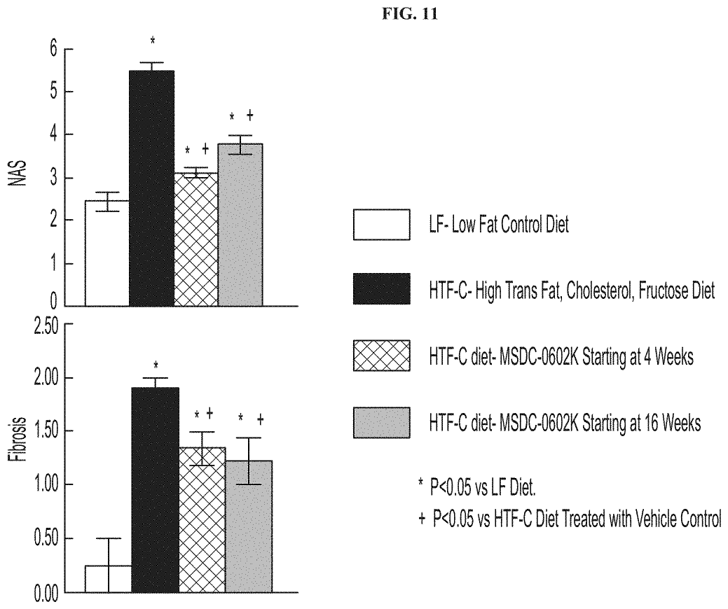

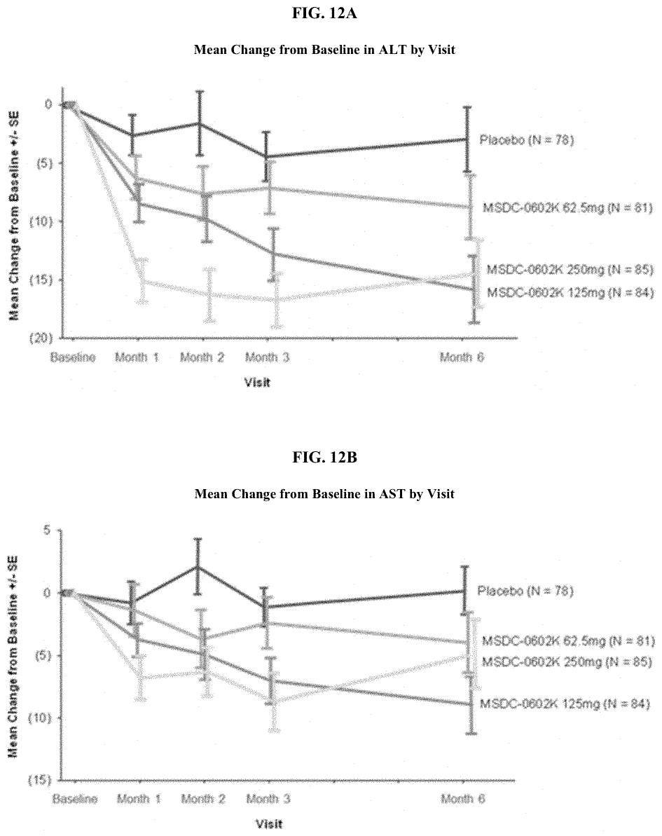

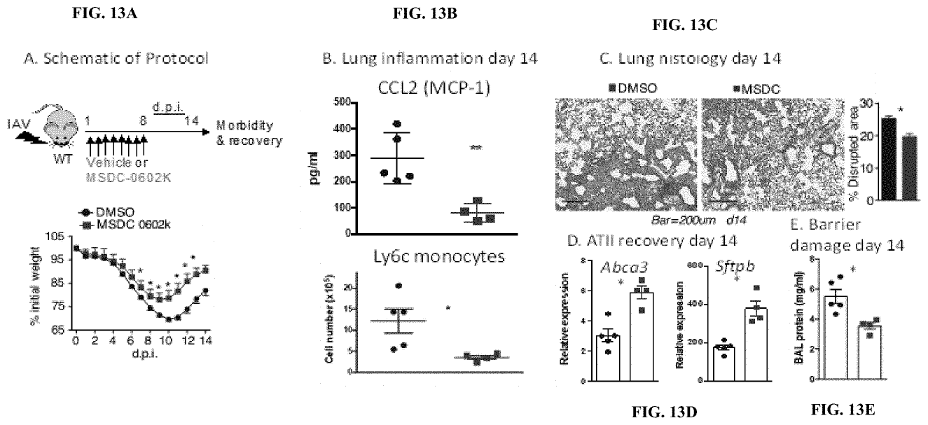

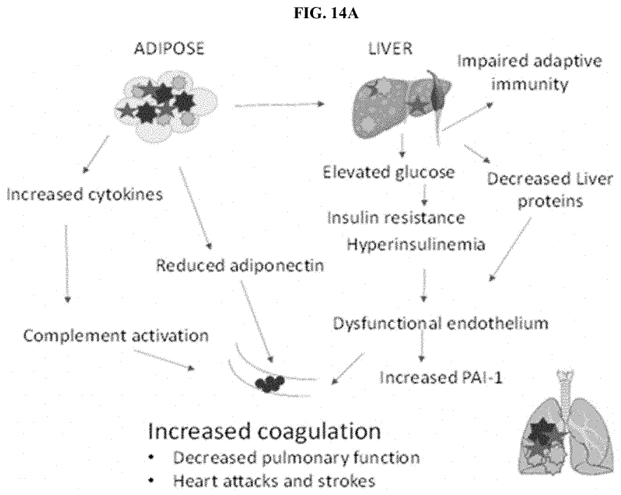

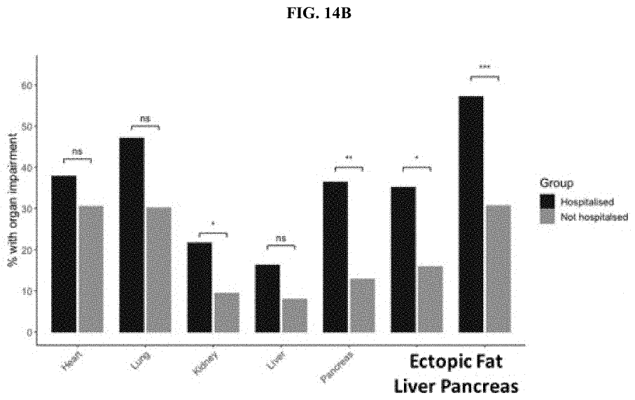

Aspects disclosed herein provide compounds with significantly reduced ability to bind to PPARγ, while maintaining interaction with the MPC. Modulation of pyruvate entry into mitochondria through the MPC exerts beneficial effects on metabolism and protects against the consequences of viral infection. FIG. 1 shows the relative binding affinities of compound MSDC-0602, the metabolite of MSDC-0602, and the two insulin sensitizers rosiglitazone and pioglitazone with PPARγ. FIG. 1 shows that pioglitazone had a more than ten-fold reduction in binding affinity for PPARγ compared to rosiglitazone, and MSDC-0602 had a more than eight-fold reduction in binding affinity for PPARγ compared to pioglitazone. Moreover, the primary metabolite of MSDC-0602, which comprises more than 90% of the combined exposure, had a more than fifty-fold reduction in binding affinity for PPARγ compared to pioglitazone. In addition to the demonstrable reduction in PPARγ binding, MSDC-0602 and the primary metabolite of MSDC-0602 maintain, and in some cases improve, binding to the MPC, as shown in FIG. 2. Thus, MSDC-0602 is a highly specific modulator of the MPC at concentrations that will not cause meaningful direct activation of PPARγ, resulting in reduced side effects and increased efficacy over the state of the art. The potassium salt, MSDC-0602K, additionally provides improved bioavailability as compared to the free acid alone.

Disclosed herein, in some embodiments are compounds of structural Formula (I), or pharmaceutically acceptable salts thereof that modulate the MPC and mitigate the effects of overnutrition and/or metabolic dysfunction implicated in metabolic disorders such as insulin resistance, prediabetes, and diabetes. In particular, administration of the compound MSDC-0602K results in a reversal of the effects of carbon delivery to the mitochondria in the form of pyruvate that exceeds energy needs. This modulation of the MPC positions MSDC-0602K upstream in the treatment of the pathophysiology of some metabolic disorders from other targets in development for the metabolic disorders. Overnutrition delivers excess pyruvate to the mitochondria through the MPC, driving changes in downstream metabolic pathways through a number of regulatory proteins, and treatment with the MPC modulator, working upstream, is able to reverse these changes.

Table 1 shows the impact of MSDC-0602K relative to overnutrition and overnutrition relative to normal function by tabulating increases and decreases in cell functions and key regulatory proteins.

TABLE 1

| | | | Impact of | Impact of |
|---|---|---|---|---|
| | | | Overnutrition v | MSDC-0602K |
| | Key Factor | Description | Normal Function | vs Overnutrition |
| Cell | Insulin | Cellular response to | Decreased | Increased |
| Functions | sensitivity | insulin | | |
| | de novo lipid | Making new fat | Increased | Decreased |
| | synthesis | | | |
| | Fatty acid | Burning Fat | Decreased | Increased |
| | oxidation | | | |
| | Inflammation | Ballooning due to | Increased | Decreased |
| | | injury | | |
| Key | mTORC1 | Important nutrient | Increased | Decreased |
| Regulatory | | sensor | | |
| Proteins | SREBP | Transcription factor | Increased | Decreased |
| | | regulating fat | | |
| | | synthesis | | |
| | PPARγ, PPARα | Transcription factors | Decreased | Increased |
| | | regulating fat | | |
| | | metabolism and | | |
| | | inflammation | | |
| | HIF1α | Transcription factor | Increased | Decreased |
| | | responding to oxygen | | |

Severe acute respiratory syndrome coronavirus 2 (SARS-CoV-2) infection response and disease severity varies greatly with age and comorbidity. For example, age, hypertension, and diabetes, and elevated body weight have been the biggest risk factors on the infection response and disease severity (based on data from China, Italy, France, and the United States of America). World figures show that 32% of COVID subjects admitted to the ICU have diabetes. COVID-19 infection has resulted in increased rates of hospitalization and greater severity of illness in people with type 1 diabetes (T1D), T2D, or obesity. See Drucker D. J., Diabetes, obesity, metabolism, and SARS-CoV-2 infection: the end of the beginning; Cell Metabolism. 2021 Mar. 2; 33(3): 479-498. The same comorbidities are also risk factors for other viral respiratory infections. The diabetes connection likely relates to insulin resistance since this relationship holds for type II diabetes, but not type I diabetes. As an MPC inhibitor insulin sensitizer, MSDC-0602K may have both long-term and acute benefits such as improved metabolism, reduced type II diabetes symptoms, or reduced immune senescence. Acute effects may include reduced viral infection and replication (e.g., by reducing mTOR activation). Acute effects may also include breaking a vicious cycle by restoring a balance to both resident and recruited macrophage function.

Furthermore, in subjects with or without obesity, disproportionate adipose tissue distribution, and particularly increased visceral adipose mass, strongly and independently predict an increased risk of cardiometabolic diseases and severity of COVID-19-related hospitalization. A study demonstrated that each 10 cm² increase in visceral adipose area measured by CT was associated with an OR of 1.37 (95% CI 1.07-1.89) for ICU treatment and an OR of 1.32 (95% CI 1.04-1.91) for mechanical ventilation (both adjusted for age and sex). Stefan N., et al., Global pandemics interconnected—obesity, impaired metabolic health and COVID-19; Nature Reviews Endocrinolofy 17, 135-149 (2021). Another study showed that for each unit of increase in the visceral adipose tissue area, measured by chest CT, there was an OR of 2.47 (95% CI 1.02-6.02) for the need for intensive care, independently of age, sex, diabetes mellitus, CVD, hypertension, kidney failure, inflammatory markers, malignancies, pharmacological treatment, total adipose tissue area and subcutaneous adipose tissue area.

Evidence supports that metabolic dysfunction (insulin resistance) may be involved in infection risk/host response. For example, animal models with insulin resistance are more prone to respiratory viral infections. Downregulation of PPARγ networks which occurs in other cells in response to overnutrition and/or metabolic dysfunction also occur in alveolar macrophages and other cells. MSDC-0602K treatment relieves this inhibition by attenuating the mitochondrial pyruvate carrier (MPC). Additionally, several potential protein targets of the SARS-CoV-2 sequence have been identified, most of which are downstream of metabolic regulation. Inhibitors of mTOR reduce Middle East respiratory syndrome (MERS) infections in vitro, and mTOR activation is downstream of MPC overaction.

The compositions described herein may provide protective effects in a subject with a respiratory infection. Metabolic effects can limit the degree of viral infection through viral entry and replication. Metabolic effects can limit the overresponse of the host tissues to the infection (this may include specialized lung cells, as well as macrophages and endothelial cells modulating the inappropriate immune and blood clotting mechanisms). Reduced insulin resistance may improve overall metabolic health, reduce the need for insulin to control glycemia, and reduce immune senescence associated with insulin resistance (see FIG. 3).

Viral entry and replication depends on metabolism in host cells (applicable across cell types). Informatics show that a number of viral proteins are suspectable to metabolic modulation by host enzymes depending on the metabolic state. Inhibitors of mTOR limit infection and replication of other RNA viruses. In the case of macrophages, the inflammatory state and response is controlled by metabolism (metabolic programming of macrophages regulates inflammatory response) (see FIG. 4). A respiratory infection such as COVID-19 may conspire with the metabolic state of the host cells to trigger an acute, deteriorating viscous cycle involving a reprogramming of resident and recruited macrophages to an exaggerated pro-inflammatory state. Treatment with MSDC-0602K should reduce mTOR activation and help restore balance (e.g., increase PPARg, PGC-1, arginase-1, KLF-4), increase the M2-type program and favor a self-limiting outcome (see FIG. 5 for examples of some embodiments).

Moreover, subjects with underlying health conditions are more vulnerable to coronavirus infections than people without an underlying health condition. For example, data on early stage case fatality rates in China indicated that 10.5% of people with a cardiovascular disease who were diagnosed with COVID-19 died, 7.3% of people with diabetes who were diagnosed with COVID-19 died, 6.3% of people with a chronic respiratory disease who were diagnosed with COVID-19 died, 6% of people with hypertension who were diagnosed with COVID-19 died, and 5.6% of people with cancer who were diagnosed with COVID-19 died, whereas only 0.9% of people without any underlying health conditions who were diagnosed with COVID-19 died.

Subjects with metabolic disorders such as diabetes and fatty liver tend to have more severe adverse respiratory outcomes when they have a respiratory infection such as a coronavirus infection. This may be due to an exaggerated inflammatory response that corresponds with the metabolic disorder. This may also involve endothelial cell dysfunction exacerbated by the interaction of metabolic issues and response to the magnitude of the viral infection. In some embodiments, administration of a composition described herein alleviates metabolic dysfunction or symptoms of a metabolic disorder, and/or reduces symptoms or progression of the respiratory infection. In some cases, the alleviation may work through a mechanism that includes reducing an exaggerated inflammatory response.

Further, subjects with acute respiratory infections may be prone to have blood clots, and MSDC-0602K may be beneficial for treatment of these patients. Endothelial damage and subsequent clotting is common in severe and critical COVID-19 coronavirus infections, which may have implications for treatment. Clots in small vessels of many organs, including lungs heart, liver, and kidney have been described in patients with COVID-19. The virus can bind to the endothelial cells and may cause damage to blood vessels, especially microcirculation of small blood vessels, which may lead to platelet aggregation. Thus, another benefit of MSDC-0602K treatment (or other compositions described herein) in subjects with a respiratory disorder may be beneficial effects on the amount of blood clots or blood clot formation.

As shown in FIGS. 14A and 114B, the long-term outcomes of COVD-19 may comprise consequent and continued hepatic and/or pancreatic injuries and impairment. FIG. 14A shows that viral infections, such as the SARS-Cov-2 infection may cause adipose tissue to secret more inflammatory cytokines, while at the same time may lead to reduced levels of adiponectin. In turn, the viral infection may leads to accumulation of fat tissue in organs, such as liver and pancreas several months after the onset of symptoms caused by, for example, the SARS-Cov-2 infection. Similarly, in FIG. 14B, when ectopic fat was measured in liver or pancreas about 140 days after the initial symptoms, the ectopic fat either in live or pancreas is significantly increased in hospitalized COVID-19 subjects (having more severe symptoms) compared to non-hospitalized COVID-19 subjects (having less severe symptoms). In addition, chronic COVID-19 effects comprise decreased quality of life, increased frailty and dyspnea, decreased sleeping quality, and etc.

In some embodiments, compounds of structural Formula (I), or pharmaceutically acceptable salts thereof are effective mediators of the effects of metabolic dysfunction. In some embodiments, compounds of structural Formula (I), or pharmaceutically acceptable salts thereof are effective modulators of the mitochondrial pyruvate carrier (MPC).

Aspects disclosed herein provide methods of treating or preventing a disease or condition associated with metabolic dysfunction, comprising administering to a subject in need thereof a therapeutically effective amount of a compound as described herein, including embodiments, or the structural Formula (I), or a pharmaceutically acceptable salt thereof. Aspects further disclosed provide methods of treating or preventing a metabolic disorder and/or a respiratory disorder, comprising administering to a subject in need thereof a therapeutically effective amount of a compound as described herein, including embodiments, or the structural Formula (I), or a pharmaceutically acceptable salt thereof. Aspects disclosed herein provide methods of treating or preventing diabetes prediabetes, insulin resistance, and/or a respiratory infection such as COVID-19, comprising administering to a subject in need thereof a therapeutically effective amount of a compound as described herein, including embodiments, or the structural Formula (I), or a pharmaceutically acceptable salt thereof. In some embodiments, the pharmaceutically acceptable salt comprises a potassium salt. In some embodiments, diabetes is Type II diabetes.

Aspects disclosed herein provide detecting a presence or a level of one or more biomarkers in a sample obtained from a subject in need thereof. The term “biomarker” as used herein refers to a measurable substance in a subject whose presence and/or level is indicative of a certain phenomenon or phenotype of the subject. A subject in need thereof may be diagnosed with, or suspected of having, a disease or condition (e.g., metabolic inflammation-mediated disease or disorder, or a respiratory disorder such as a coronavirus infection) disclosed herein. In some instances, a decrease in a level of the one or more biomarkers in a sample obtained from the subject, as compared to a level of the one or more biomarkers (e.g., a “baseline” or “control” level) in a sample obtained from an individual, or group of individuals, that is not diagnosed with the disease or condition, is detected. In some instances, an increase of the one or more biomarkers in a sample obtained from the subject, as compared to a baseline level of the one or more biomarkers in a sample obtained from an individual, or group of individuals, that was not diagnosed with the disease or condition is detected. In some instances, the baseline level of the one or more biomarkers is determined using a single individual, two or more individuals, or a plurality of individuals who were not diagnosed with the disease or condition (e.g., a “normal” individual). In some instances, the sample comprises whole blood, peripheral blood, plasma, serum, urine, saliva, or other biological sample. In some instances, the one or more biomarkers comprises protein, ribonucleic acid (RNA), or deoxyribonucleic acid (DNA), or a combination thereof. In some instances, the one or more biomarkers comprises alanine transaminase (ALT), aspartate aminotransferase (AST), homeostatic model assessment (HOMA) (a method for assessing (3-cell function and insulin resistance (IR) from basal (fasting) glucose and insulin or C-peptide concentrations (HOMA-IR)), and/or hemoglobin lac (HbA1c).

In some instances, the one or more biomarkers comprises a coronavirus peptide, a coronavirus nucleic acid, a COVID-19 peptide, a COVID-19 nucleic acid. In some instances, the one or more biomarkers comprises a coronavirus peptide. In some instances, the one or more biomarkers comprises a coronavirus nucleic acid. In some instances, the one or more biomarkers comprises a COVID-19 peptide. In some instances, the one or more biomarkers comprises a COVID-19 nucleic acid. The coronavirus peptide or the COVID-19 peptide may be an envelope protein. The coronavirus nucleic acid or the COVID-19 nucleic acid may be an RNA. Some examples of such include an RNA fragment of the coronavirus or COVID-19 RNA genome, or an RNA transcript transcribed from a reverse transcribed DNA sequence. The coronavirus nucleic acid or the COVID-19 nucleic acid may include a DNA sequence (e.g., a reverse transcribed DNA sequence from the coronavirus or COVID-19 RNA genome. In some cases, the DNA or RNA biomarker is assessed by PCR (e.g. RT-PCR or RT-qPCR). In some cases, the DNA or RNA biomarker is assessed by sequencing.

ALT and AST are biomarkers for liver injury and disease. Non-limiting examples of biomarkers of injury include aminotransferases (e.g., ALT and AST), γ-glutamyl transferase (GGT), alkaline phosphatase (ALP), lactate dehydrogenase (LDH), and glutamate dehydrogenase (GLDH). Non-limiting examples of biomarkers of function include prothrombin time, and bilirubin. Non-limiting examples of bio markers of proliferation include α-fetoprotein. A normal range, or baseline, of ALT may be about 6 to about 34 units per liter (U/L) for women and about 6 to about 43 U/L for men. A normal range, or baseline, of AST is about 6 to about 34 U/L for women, and about 9 to about 36 U/L for men. A level of ALT that is above about 34 U/L and 43 U/L for a woman subject and a man subject, respectively, indicates that the subject either has, or will develop, a liver disease or condition (e.g., NASH, NAFLD). A level of AST that is above about 34 U/L and 36 U/L for a woman subject and a man subject, respectively, indicates that the subject either has, or will develop, a liver disease or condition (e.g., NASH, NAFLD).

HbA1c is a biomarker of blood sugar level during the previous two to three months that is used to diagnose and monitor diabetes. Normal adult hemoglobin consists predominantly of HbA (α2β2), HbA2 (a2δ2), and HbF (α2γ2) in the composition of 97%, 2.5%, and 0.5%, respectively. About 6% of total HbA is termed HbA1, which in turn is made up of HbA1a1, HbA1a2, HbA1b, and HbA1c fractions, defined by their electrophoretic and chromatographic properties. HbA1c is the most abundant of these fractions and in health comprises approximately 4-5% of the total HbA fraction. Disclosed herein, in some embodiments, is a normal range, or baseline level, of HbA1c of between 4% and 5.6% in an individual who does not have diabetes. Further provided are levels of HbA1c between 5.7% and 6.4% in a subject that indicate an increased likelihood that the subject will develop diabetes, whereas levels of HbA1c of about 6.5% and higher in a subject indicate that the individual has diabetes.

Aspects disclose herein provide methods of treating a subject in need thereof by administering to the subject a compound as described herein, including structural Formula (I), provided an increase in a level of AST and/or ALT is detected in a sample obtained from the subject, as compared to a level of AST and/or ALT in a normal individual. In some instances, the increase in the level of ALT comprises above about 34 units per liter (U/L) for woman and above about 43 U/L for a man. In some instances, the increase in the level of AST comprises above about 34 U/L for a woman, and above about 36 U/L for a man. In some instances, the subject in need thereof suffers from a metabolic inflammation-mediated disease or disorder. In some instances, the subject suffers from diabetes, including Type II diabetes. In some instances, the subject suffers from prediabetes. In some instances, the subject suffers from insulin resistance. In some instances, the subject suffers from COVID-19.

Aspects disclose herein provide methods of treating a subject in need thereof by administering to the subject a compound as described herein, including structural Formula (I), provided an increase in a level of HbA1c is detected in a sample obtained from the subject, as compared to a level of HbA1c in a normal individual. In some instances, the increase in the level of HbA1c comprises above about 6.5%. In some instances, the subject in need thereof suffers from a metabolic inflammation-mediated disease or disorder. In some instances, the subject suffers from diabetes, including Type II diabetes. In some instances, the subject suffers from prediabetes. In some instances, the subject suffers from insulin resistance. In some instances, the subject suffers from COVID-19.

Aspects disclosed herein also provide methods of monitoring a progression of a treatment of a subject with the compounds disclosed herein, including structural Formula (I). Also disclosed are methods of optimizing a treatment for a subject with the compounds disclosed herein, including structural Formula (I). In some instances, monitoring and/or optimizing the treatment comprises detecting a level of the one or more biomarkers disclosed herein, including ALT, AST, and/or HbA1c in a sample obtained from the subject. In some instances, the one or more biomarkers comprises protein, RNA, and/or DNA. In some instances, a decrease in the level of ALT, AST, and/or HbA1c in a sample obtained from the subject indicates the treatment of the subject is efficacious.

### VI. EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

### Example 1. Acute Mitochondrial Pyruvate Carrier-Dependent and -Independent Effects of MSDC-0602 in Cells of Mice with Metabolic Disorders

Insulin-sensitizing thiazolidinediones (TZDs) have shown promise for the treatment of metabolic disorders, but their use is limited by side effects of PPARγ-agonism (such as effects on fluid balance which are particularly an issue in subjects with respiratory disorders). MSDC-0602 is a “next-generation” TZD which does not bind/activate PPARγ, but binds and modulates the mitochondrial pyruvate carrier (MPC). It has been reported that treatment with MSDC-0602 prevents and reverses damage caused to the liver in a mouse model of a metabolic disorder, and that these beneficial effects require MPC expression in hepatocytes. A purpose of these studies were to differentiate the acute MPC2-dependent and -independent effects of MSDC-0602. A follow-up study to investigate the circulating exosome miRNA content in a mouse model of a metabolic disorder with and without MSDC-0602 treatment was then performed.

Methods

8-week-old WT and liver specific MPC2−/− LS-MPC2−/− mice were fed a diet of 60% fat (Research Diets D12492) for 20 weeks to induce obesity and insulin resistance. Intraperitoneal glucose tolerance tests (GTT) were performed after a 4 h fast by injection of 1 g/kg D-glucose in saline i.p. to assess glucose tolerance. 3 days after the initial GTT, mice were randomized to receive a single gavage of either vehicle (1% CMC, 0.01% Tween-80) or 30 mg/kg MSDC-0602K. Plasma insulin was measured by Singulex assay and plasma ALT levels were measured by commercially-available kits (Teco Diagnostics). qPCR was performed by isolation of total RNA with RNA-Bee and reverse transcription with a high-capacity cDNA synthesis kit (Invitrogen). qPCR was performed on an Applied Biosystems real-time thermocycler.

Metabolic Disorder (HTF-C) Diet Plasma RNA Experiment Results

To begin evaluating the exosome cargo of MSDC-0602K-treated mice, WT mice were fed with either a low fat (LF) control diet (Research Diets D09100304) or high trans-fat, fructose, cholesterol (HTF-C) diet composed of 40% trans-fat, 20% fructose, 2% cholesterol (Research Diets D09100301). A subset of mice was fed plain HTF-C diet for 4 or 16 weeks, then switched to HTF-C diet that contained 331 ppm MSDC-0602K.

Total RNA was isolated from 400 μL of serum and small RNA sequencing was performed by adapter ligation, cDNA synthesis and size selection of 145-160 base pairs. Sequencing was then performed on an Illumina HiSeq3000.

It was found that diet-induced obese LS-MPC2−/− mice display improved glucose tolerance. WT (fl/fl) and LS-MPC2−/− mice were put on 60% HF diet and became equally obese. A GTT reveals that obese LS-MPC2−/− mice are more glucose tolerant. (FIGS. 6A-6C).

Moreover, a single dose of MSDC-0602K improves glucose tolerance. GTT 20 h after a single dose of vehicle or 30 mg/kg MSDC-0602K shows improved glucose tolerance in WT mice treated with MSDC-0602. LS-MPC2−/− again show improved glucose tolerance compared to WT mice, and appear to show no improvement in glucose tolerance. However, plasma insulin values are decreased in both WT and LS-MPC2−/− mice that were treated with MSDC-0602K, indicating that both WT and LS-MPC2−/− mice have improved insulin sensitivity after MSDC-0602K treatment. (FIGS. 7A-7C).

Liver-specific MPC2 deletion or acute MSDC-0602K diminishes markers of liver injury in a metabolic disorder. LS-MPC2−/− mice, or WT mice treated with a single dose of MSDC-0602K display decreased plasma ALT concentrations (FIG. 8A) and decreased gene expression for hepatic stellate cell activation and fibrotic scar formation (FIG. 8B). For these analyses, LS-MPC2−/− mice appear refractory to the beneficial effects of acute MSDC-0602K treatment.

Altering hepatocyte metabolism by MPC2−/− or MSDC-0602K treatment regulates exosome signaling to hepatic stellate cells as shown in FIG. 9.

Serum miRNAs are altered in a mouse model of a metabolic disorder and largely corrected by MSDC-0602K treatment. Heat map of serum miRNAs depicts large number of counter-regulated miRNAs with HTF-C diet and treatment with MSDC-0602K. Heat map miRNAs (˜230 miRNAs) were selected by filtering data for 2-fold or greater change and FDR<0.1 comparing LF to HTF-C diet (not shown). Examples of miRNAs that are unregulated in the metabolic disorder and down with MSDC-0602K (FIGS. 10A-10C), down in the metabolic disorder and up with MSDC-0602K treatment (FIGS. 10D-10F), or simply show large effect of MSDC-0602K treatment (FIGS. 10G-10H). Dysregulation of a number of these miRNAs has been previously identified in metabolic disorders.

### Conclusions

It was found that liver-specific KO of MPC improves insulin sensitivity in diet-induced obesity. Furthermore, acute dosing with MSDC-0602K improves glucose tolerance, but is not totally dependent on hepatocyte MPC. The major acute effects of MSDC-0602K to attenuate liver injury involved hepatocyte MPC and included release of factors from hepatocytes that affect stellate cells. Altering hepatocyte pyruvate metabolism regulates exosome cargo, which can alter the activation and fibrogenesis of hepatic stellate cells.

### Example 2. Additional Preclinical Studies of Metabolic Disorders

MSDC-0602K was tested in multiple animal models of a metabolic disorder and has been shown to protect against the high fat, high cholesterol, high sugar diet-induced metabolic dysfunction. Mice were fed a diet enriched in trans fat, cholesterol and fructose for 19 weeks to induce hepatic damage and metabolic dysfunction, which was assessed by histological changes in the NAS and fibrosis, measured by trichrome staining for collagen. MSDC-0602K (30 mg/kg) was given at either four weeks into the treatment or 16 weeks into the treatment as compared to the vehicle treated mice who were also on the modified diet. The results of mice who were kept on the normal diet are included for comparison. FIG. 11 shows the comparison between mice kept on the normal diet and mice described above. The results show that the setting of metabolic dysfunction, signals from hepatocytes can activate stellate cells, which are involved in the initiation of fibrosis. The signals for activation of the stellate cells and fibrosis are attenuated by treatment of the mice or isolated hepatocytes with MSDC-0602K.

### Example 3. Interim Analysis of the EMMINENCE Phase 2b Clinical Trial

An interim analysis of the EMMINENCE Phase 2b trial was conducted in the first 328 subjects who reached their six-month follow-up visit. This study is a randomized, double-blinded study of three doses (62.5 mg 125 mg, or 250 mg) of MSDC-0602K or placebo given orally once daily to subjects with biopsy proven non-alcoholic steatohepatitis (NASH; a non-limiting example of a metabolic disorder) with fibrosis and no cirrhosis.

The interim analysis of explanatory endpoints showed statistically significant reductions in liver enzymes, including ALT and AST, measured at six months compared to baseline. In the two highest dose groups, at least 50% of patients with elevated baseline Alanine transaminase (ALT) or Aspartate Aminotransferase (AST) improved into the normal range at six months, as shown in Table 3. Statistically significant reductions in homeostatic model assessment (HOMA), a method for assessing (3-cell function and insulin resistance (IR) from basal (fasting) glucose and insulin or C-peptide concentrations (HOMA-IR) and Hemoglobin lac (HbA1c) were also observed in all MSDC-0602K dose cohorts. Overall adverse event rates were similar across placebo and all doses of MSDC-0602K.

The subjects included in this interim analysis had significant liver disease, as established by liver biopsy, with an average NAS at baseline of 5.3. Almost 60% of these subjects had a baseline fibrosis score of F2 or F3 and approximately 50% also had a diagnosis of Type II diabetes at baseline. Overall, baseline characteristics were well-balanced across treatment groups. Table 2 provides baseline characteristics of subjects included in the interim analysis.

TABLE 2
Baseline Characteristics of Subjects Included in Interim Analysis

| | | | MSDC-0602K | | |
|---|---|---|---|---|---|
| All Subjects | | Placebo | 62.5 mg | 125 mg | 250 mg |
| (n = 328): | | (n = 78) | (n = 81) | (n = 84) | (n = 85) |
| Age (mean, years) | | 54.4 | 56.7 | 56.3 | 57.5 |
| Male | | 47.4% | 39.5% | 35.7% | 42.4% |
| Female | | 52.6% | 60.5% | 64.3% | 57.6% |
| Weight (mean, kg) | | 102.6 | 96.2 | 99.2 | 98.2 |
| Type 2 Diabetes | | 50.0% | 53.4% | 52.4% | 51.8% |
| Present | | | | | |
| ALT (mean, U/L) | | 59.3 | 58.6 | 49.8 | 58.0 |
| AST (mean, U/L) | | 42.4 | 45.2 | 42.7 | 44.8 |
| | Diabetic Subjects | Placebo | 62.5 mg | 125 mg | 250 mg |
| | (n = 170): | (n = 39) | (n = 45) | (n = 43) | (n = 43) |
| | HbA1c | 6.83% | 7.09% | 6.90% | 6.97% |

FIGS. 12A-12B show the levels of change from baseline in ALT and AST by visit over 6 months of treatment with MSDC-0602K (62.5 mg, 125 mg, 250 mg). Statistically significant placebo-corrected reductions at six months in ALT levels were observed in the 125 mg and 250 mg dose cohorts (FIG. 12A). A statistically significant placebo-corrected reduction at six months in AST levels was observed in the 125 mg dose cohort (FIG. 12B). Placebo-corrected reductions at six months of 14.3 Units/Liter, U/L, (p<0.001) and 7.9 U/L (p=0.012) in ALT and AST, respectively, in the 125 mg cohort, and 10.6 U/L (p=0.004) and 4.0 (not significant) in ALT and AST, respectively, in the 250 mg cohort. Placebo-corrected reductions, relative to baseline, were 25% and 18% in ALT and AST, respectively, in the 125 mg cohort, and 19% and 9% in ALT and AST, respectively, in the 250 mg cohort. Importantly, normalization of ALT and AST was observed across all three dose levels of MSDC-0602K, with the two highest dose groups having at least 50% of subjects improved to normal range. ALT normal range is defined as 6 to 34 U/L and 6 to 43 U/L for women and men, respectively, and AST normal range is defined as 9 to 34 U/L and 11 to 36 U/L for women and men, respectively. Table 3 shows the percentage of patients with high baseline values who returned to normal values.

TABLE 3
Percentage of Patients with High Baseline ALT
and AST Values who Returned to Normal Range

| | | MSDC-0602K | | |
|---|---|---|---|---|
| | Placebo | 62.5 mg | 125 mg | 250 mg |
| ALT | 15% | 29% | 60% | 56% |
| AST | 20% | 36% | 50% | 52% |

Improvements were also observed in bilirubin, alkaline phosphatase, and gamma GT liver enzymes. Across all subjects, statistically significant improvements were observed at six months in fasting glucose, HbA1c (Table 4), insulin levels, and HOMA-IR at 125 mg and 250 mg doses of MSDC-0602K. Among Type II diabetes subjects, for whom mean baseline values for HbA1c were relatively low compared to populations in clinical trials of other Type II diabetes therapies, statistically significant improvement was observed in HbA1c at all doses of MSDC-0602K. Adiponectin increased significantly in all treatment cohorts in a dose dependent manner, and there was a decrease in C-Reactive Protein levels seen in the two lowest doses. Table 4 shows HbA1c measurements in subject with Type II Diabetes at baseline and six months. Also observed were trends for improvement in additional biomarkers of fibrosis such as hematocrit and, overall, a neutral effect on lipids.

TABLE 4
Mean HbA1c Measurements in Subjects with Type
II Diabetes at Baseline and Six Months

| | | MSDC-0602K | | |
|---|---|---|---|---|
| | Placebo | 62.5 mg | 125 mg | 250 mg |
| Baseline | 6.83% | 7.09% | 6.90% | 6.97% |
| Placebo-corrected | | ▾0.37 | ▾0.55 | ▾0.45 |
| change from Baseline | | | | |
| at 6 months | | | | |
| p-value | | 0.022 | 0.001 | 0.006 |

The overall rates of treatment emergent adverse events were similar across placebo and all MSDC-0602K cohorts. Importantly, the frequency of any degree of peripheral edema, a finding associated with direct PPARγ agonism, observed at six months was similar to that observed at baseline and was comparable across placebo and MSDC-0602K cohorts. Table 5 provides peripheral edema present at baseline and at six months. The reduction in edema indicates better fluid handling in subjects treated with MSDC-0602K, and thus, that subjects with a respiratory disorder such as COVID-19 may benefit from treatment with MSDC-0602K. The MSDC-0602K may reduce or not worsen peripheral edema shows that MSDC-0602K may be used when the use of direct PPARγ agonists may be contraindicated (such as during an acute respiratory infection such as COVID-19).

TABLE 5
Peripheral Edema Present at Baseline and Six Months

| | | MSDC-0602K | | | |
|---|---|---|---|---|---|
| | Placebo | 62.5 mg | 125 mg | 250 mg | Total |
| | (n = 78) | (n = 81) | (n = 84) | (n = 85) | (n = 328) |
| Baseline | 7.70% | 13.60% | 13.10% | 9.40% | 11.00% |
| 6 months | 12.50% | 7.80% | 13.50% | 11.10% | 11.20% |

To preserve the blinding of this ongoing clinical trial, adverse events that were reported by fewer than five subjects in an individual cohort were not unblinded as to distribution across cohorts. Treatment emergent serious adverse events were reported in fourteen subjects, none of which were determined by the investigator to be drug-related.

Additionally, MSDC-0602K treatment reduced Branched Chain Amino Acids [BCAA, baseline to 12-month endpoint (Visit 7)] (see Table 6). Higher levels of BCAA can correlate with blood clotting risks. Since subjects with acute respiratory infections may be prone to have blood clots, MSDC-0602K may be beneficial.

TABLE 6

| | | TotalBCAA | | | Valine | | | Leucine | | | Isoleucine | | |
|---|---|---|---|---|---|---|---|---|---|---|---|---|---|
| | | | | Change | | | Change | | | Change | | | Change |
| | | | | from | | | from | | | from | | | from |
| | | Baseline | Visit 7 | Baseline | Baseline | Visit 7 | Baseline | Baseline | Visit 7 | Baseline | Baseline | Visit 7 | Baseline |
| Placebo | MEAN | 427.12 | 429.38 | −1.87 | 237.83 | 236.17 | −4.24 | 124.52 | 127.22 | 1.94 | 64.77 | 65.99 | 0.43 |
| N = 94 | SD | 80.70 | 92.09 | 78.93 | 42.61 | 45.23 | 39.17 | 30.77 | 37.71 | 34.09 | 16.62 | 17.80 | 19.01 |
| | SEM | 8.32 | 9.50 | 8.14 | 4.40 | 4.66 | 4.04 | 3.17 | 3.89 | 3.52 | 1.71 | 1.84 | 1.96 |
| 62 5 mg | MEAN | 430.03 | 397.39 | −30.07 | 239.20 | 221.29 | −16.74 | 121.80 | 114.63 | −6.74 | 69.03 | 61.47 | −6.58 |
| N = 99 | SD | 89.26 | 71.41 | 83.87 | 48.09 | 36.21 | 37.32 | 30.24 | 30.11 | 34.56 | 20.49 | 14.23 | 22.85 |
| | SEM | 8.97 | 7.18 | 8.43 | 4 83 | 3.64 | 3.75 | 3.04 | 3.03 | 3.47 | 2.06 | 1.43 | 2.30 |
| 125 mg | MEAN | 408.60 | 399.74 | −9.61 | 230.43 | 223.73 | −6.08 | 115.62 | 115.95 | −0.60 | 62.55 | 60.06 | −2.92 |
| N = 9B | SD | 86.62 | 99.23 | 97.07 | 44.94 | 51.30 | 48.17 | 33.26 | 37.49 | 39.85 | 17.73 | 18.67 | 22.10 |
| | SEM | 8.75 | 10.02 | 9.81 | 4.54 | 5.18 | 4.87 | 3.36 | 3.79 | 4.02 | 1.79 | 1.89 | 2.23 |
| 250 mg | MEAN | 420 61 | 378 34 | −37.80 | 232.15 | 212.52 | −17.15 | 121.54 | 109 67 | −10.55 | 66.92 | 56.15 | −10.10 |
| N = 101 | SD | 99.32 | 88.20 | 91.52 | 44.39 | 44.75 | 42.15 | 41.66 | 35.59 | 37.91 | 21.35 | 15.10 | 22.78 |
| | SEM | 9.93 | 8.82 | 9.15 | 4.44 | 4.48 | 4.21 | 4.17 | 3.56 | 3.79 | 2.14 | 1.51 | 2.28 |

### Example 4. Preclinical Studies of Respiratory Disorders

Studies are performed in pre-clinical animal models of respiratory disorders including coronavirus disease 2019 (COVID-19), severe acute respiratory syndrome (SARS), and Middle East respiratory syndrome (MERS). Animals such as hamsters or monkeys are infected with a coronavirus such as severe acute respiratory syndrome coronavirus (SARS-CoV), severe acute respiratory syndrome coronavirus 2 (SARS-CoV-2), or Middle East respiratory syndrome coronavirus (MERS-CoV), or a mock-infection control. In each set of animals with the infection or mock-infection, animals are administered an effective dose of MSDC-0602K, pioglitazone, or a control treatment comprising a vehicle without either drug. Respiratory infection endpoints are analyzed, including respiratory function, respiratory inflammation, and viral load.

A larger study is performed to identify interactions for 3 variables (infection, metabolic dysfunction, and drug treatment) on respiratory and metabolic outcomes. The metabolic dysfunction is induced by a high fat and high sugar diet, and compared to a control diet. Metabolic outcomes include measures of insulin resistance and glucose tolerance. MSDC-0602K is expected to outperform pioglitazone on respiratory and metabolic outcomes in the animals with coronavirus infections and/or the high fat and high sugar diet.

As shown in the top panel of FIG. 13A, C₅₇BL/6 mice were infected with the influenza virus A/PR/8/34 (H1N1; ˜200 pfu/mouse) and then dosed for 8 days with vehicle or 30 mg/kg MSDC-0602K. As shown in the bottom panel of FIG. 13A, body weight was measured daily as indication of the health of the mice and at day 14 the mice were sacrificed for examination of their blood and lung tissue. The mice were sacrificed on day 14. FIG. 13B shows the MCP-1 (CCL2) production in the lungs (upper panel) and inflammatory monocyte numbers in the lungs (lower panel). The MCP-1 production levels are decreased in mice treated with MSDC-0602K compared with mice with vehicle treatment. Similarly, the inflammatory monocyte numbers in the lungs are reduced in mice treated with MSDC-0602K compared with mice with vehicle treatment. FIG. 13 C is a representative histology with a calculation of the percent disrupted area. The disrupted area in lung was significantly reduced in mice treated with MSDC-0602K compared with mice with vehicle treatment. FIG. 14D shows the expression of alveolar type II cell markers surfactant protein-B (sftbp) and ABCA3 protein (abca3) in the lungs and FIG. 13E shows total protein levels in bronchoalveolar lavage (BAL, reflective of lung barrier leakage). The levels of protein-B (sftbp) and ABCA3 protein (abca3) were increased in mice treated with MSDC-0602K compared with mice with vehicle treatment. On the other hand, the total protein levels in bronchoalveolar lavage were decreased in mice treated with MSDC-0602K compared with mice with vehicle treatment.

These data show that treatment of infected mice improved recovery from infection in a process that included reduced inflammation in the lunges both in terms of the levels of activated cytokine CCL2 and the recruitment of inflammatory monocytes (FIG. 13B). There was less damage to the lung both in terms of histology and leakage (FIGS. 13C and E) and the functionality of the alveolar type II cells that are responsible for production of surfactant needed for lung function.

### Example 5: Formulation of MSDC-0602K

Described below in Table 7 is a non-limiting example of a tablet form of MSDC-0602K.

TABLE 7

| | Amount per tablet | | | | |
|---|---|---|---|---|---|
| | (mg/tablet) | | | | Quality |
| Component | 62.5 mg | 125 mg | 250 mg | Function | Standard |
| MSDC-0602K, Int. Spec. | 68.900 | 137.800 | 275.600 | Active | GMP |
| | | | | Pharmaceutical | |
| | | | | ingredient | |
| Lactose Anhydrous | 65.525 | 131.050 | 262.100 | Diluent | NF |
| (SuperTab 21 AN) | | | | | |
| Magnesium Stearate | 0.700 | 1.400 | 2.800 | Lubricant | NF |
| Weight of Granules: | 135.125 | 270.250 | 540.500 | | |
| Microcrystalline Cellulose | 29.500 | 59.000 | 118.00 | Diluent | NF |
| (Avicel PH 200) | | | | | |
| Croscarmellose Sodium | 3.375 | 6.750 | 13.500 | Disintegrant | NF |
| (Ac-Di-Sol) | | | | | |
| Povidone K-30 | 5.250 | 10.500 | 21.00 | Binder | USP |
| (PVP K-30) | | | | | |
| Colloidal Silicon Dioxide | 0.825 | 1.650 | 3.300 | Glidant | NF |
| (Aerosil 200) | | | | | |
| Magnesium Stearate | 0.925 | 1.850 | 3.700 | Lubricant | NF |
| Core Tablet Weight: | 175.000 | 350.000 | 700.000 | | |
| Opadry White (YS-1- | 5.250 | 10.50 | 21.000 | Film Coating | |
| 7003), Int. Spec. | | | | Agent | |
| Opadry composition³ | | | | | |
| Titanium Dioxide | 1.641 | 3.282 | 6.565 | Opacifier | USP |
| Hypromellose 2910 3 cP | 1.569 | 3.137 | 6.275 | Film forming | USP |
| | | | | agent | |
| Hypromellose 2910 6 cP | 1.569 | 3.137 | 6.275 | Film forming | NF |
| | | | | agent | |
| Polyethlene Glycol 400 | 0.420 | 0.840 | 1.680 | Plasticizer | NF |
| Polysorbate 80 | 0.053 | 0.105 | 0.210 | Wetting agent | NF |
| Purified Water¹ | | | | Coating solvent | USP |
| Total Average Coated | 180.250 | 360.500 | 721.000 | — | — |
| Tablet Weight | | | | | |

¹Water removed during manufacturing

### Example 6. Clinical Studies of Respiratory Disorders

A short-term clinical trial with MSDC-0602K could provide direct evidence of potential utility. For example, pioglitazone, the only insulin sensitizer available, is generally recommended to be discontinued during respiratory infections due to issues with fluid accumulation.

Studies are performed in patients with coronavirus infections and metabolic disorders such as diabetes, prediabetes, fatty liver, or insulin resistance syndrome, to compare the efficacy and side-effects of MSDC-0602K and pioglitazone. A clinical trial is conducted on the efficacy and safety of MSDC-0602K in the treatment of patients with COVID-19. Patients are stratified based on whether they have metabolic disorders such as type II diabetes, insulin resistance, or prediabetes, so the efficacy and safety of MSDC-0602K may be evaluated in subjects with and without underlying metabolic disorders, and so the efficacy and safety of MSDC-0602K may be compared between subjects with and without the underlying metabolic disorders.

Overall, treatment with MSDC-0602K is expected to provide superior performance and decreased adverse respiratory side-effects compared to pioglitazone treatment or a placebo control.

Detailed Clinical Trial Description

This study is a multi-center, randomized, open, blank-controlled, multi-stage clinical study. The first phase assesses the efficacy and safety of MSDC-0602K compared to standard treatment for approximately 600 hospitalized adult patients diagnosed with COVID-19.

Patients with COVID-19 within 7 days of onset of symptoms are screened and randomly assigned as soon as possible after screening (within 24 hours). Patients are allocated in a 1:1 ratio, receiving the MSDC-0602K treatment or only the standard treatment. Patients who do not meet the inclusion and exclusion criteria are only allowed to be re-screened once, provided that the time from symptom onset to randomization remains within 7 days.

The MSDC-062K treatment includes orally taking 2 daily MSDC-0602K capsules, each containing 125 mg of MSDC-0602K. Alternative study designs include recruiting more patients to compare the following doses within the COVID-19 population: a MSDC-0602K treatment that includes orally taking 1, 2, or 3 daily MSDC-0602K capsules, each containing 62.5 mg, 125 mg, or 250 mg of MSDC-0602K. The standard treatment includes palliative care, and routine or emergency hospital care to deal with COVID-19 symptoms. The treatment course is for 10 days. Alternatively, the treatment course could be anywhere between 5 and 30 days, or longer.

The primary endpoints are the incidence of side-effects within 14 days of enrollment. Alternatively, the primary endpoint could be determined anywhere within a time frame between 5 and 35 days, or longer as shown below:
Primary Outcome Measures:
   1. The incidence of side effects [Time Frame: Within 14 days after enrollment]
      dyspnea
   2. The incidence of side effects [Time Frame: Within 14 days after enrollment]
      SPO2≤94%
   3. The incidence of side effects [Time Frame: Within 14 days after enrollment]
      respiratory rate≥24 breaths/min in oxygen state)
Secondary Outcome Measures:
   1. Time from patient enrollment to clinical remission [Time Frame: Within 14 days after enrollment]
      the patient had a normal body temperature of >for 24 hours (without taking antipyretic drugs or hormones) without self-consciousness Dyspnea or reduced dyspnea;
   2. Proportion of patients with normal body [Time Frame: Within 14 days after enrollment]
      Proportion of patients with normal body
   3. Proportion of patients without dyspnea [Time Frame: Within 14 days after enrollment]
      Proportion of patients without dyspnea
   4. Proportion of patients without cough [Time Frame: Within 14 days after enrollment]
      Proportion of patients without cough
   5. Proportion [Time Frame: Within 14 days after enrollment]
      Proportion of patients without oxygen treatment
   6. The negative conversion rate of SARS-CoV-2 nucleic acid [Time Frame: Within 14 days after enrollment]
      The negative conversion rate of SARS-CoV-2 nucleic acid
   7. Proportion [Time Frame: within 28 days after enrollment]
      Proportion of patients hospitalized/hospitalized in ICU
   8. Frequency of serious adverse drug events. [Time Frame: within 28 days after enrollment]
      Frequency of serious adverse drug events.

Inclusion criteria are as follows:
Age≥18 years;
Clinically diagnosed patients with COVID-19, including: in accordance with the criteria for suspected cases, have one of the following etiology evidences:
   Real-time fluorescence RT-PCR of respiratory specimens or blood specimens for detection of SARS-CoV-2 nucleic acid;
   Sequencing of viral genes in respiratory specimens or blood specimens, highly homologous to SARS-CoV-2
The time interval between the onset of symptoms and random enrollment is within 7 days. The onset of symptoms is mainly based on fever. If there is no fever, cough, diarrhea or other related symptoms can be used.

Exclusion criteria are as follows:
Any situation where the program cannot be carried out safely;
Patients who have used MSDC-0602K;
No clinical manifestations and chest imaging findings;
Known allergy or hypersensitivity to MSDC-0602K;
Disabled in patients with uncontrolled autoimmune diseases;
Patients with severe heart disease, decompensated liver disease, renal insufficiency (CrCL<50 ml/min), or those with abnormal bone marrow function;
Epilepsy and impaired central nervous system function;
Pregnancy: positive pregnancy test for women of childbearing age;
Breastfeeding women who have not stopped breastfeeding.

### Example 7. Reducing COVID-19 Disease Severity by Targeting Metabolic Dysfunction with MSDC-0602K

Host response to infection with SARS-CoV-2 differs markedly depending on age and pre-existing conditions. While up to 80% of those infected may present with mild to moderate symptoms, including asymptomatic carriers, many people experience a fulminant disease course that requires hospitalization, intensive care, and may lead to significant end organ damage and mortality. In patients with diabetes mellitus, COVID-19 disease is associated with deterioration of glycemic control. In parallel, diabetes mellitus is associated with worse outcomes in patients infected with the virus. Evidence suggests that both glycemic control and insulin resistance are key drivers of the COVID-19 disease pathology.

Global experience with COVID-19 disease continues to demonstrate that a more severe course is associated with older age, obesity, and pre-existing conditions including diabetes, hypertension, cardiovascular disease, and fatty liver. In people with diabetes, the severity of symptoms and outcomes are correlated with the hemoglobin A1c levels. Furthermore, elevated blood glucose has been associated with adverse COVID-19 outcomes even in those without diabetes. In fact, it has recently been shown that increasing blood glucose levels in vitro promote the efficiency and effectiveness of SARS-CoV-2 infection of monocytes and the inflammatory response to infection through an effect of increased glycolysis on HIF-1α signaling. Supportive of the importance of glucose levels per se on the COVID-19 disease pathophysiology, a small study of hospitalized people with diabetes and COVID-19 disease showed that intravenous insulin to control hyperglycemia improved short-term outcomes. However, the insulin infusion did not ameliorate the inflammatory response and the elevated levels of D-dimers, which may contribute to long-term adverse events related to the progressive inflammatory and coagulation pathology.

Generalized metabolic dysfunction associated with underlying insulin resistance is also exacerbated by COVID-19 disease and likely plays an important role in the host response. Insulin resistance is highly prevalent in people who are obese and particularly in people who also have pre-existing cardiometabolic conditions (such as cardiovascular disease and fatty liver) that put them at the greatest risk for adverse outcomes when infected with SARS-CoV-2 (13-17). The underlying metabolic dysfunction includes decrease in the functionality of white adipose tissue characterized by inflammation and reduced neutral lipid storage in adipose cells. This in turn results in the deposition of lipids in other tissues and immune and vascular pathology. The metabolic dysfunction in the adipose cells may also occur in adipose-like cells in the lungs contributing to increased fibrosis and adverse response to the infection. Because of these interactions between insulin resistance and dysfunctional adipose tissue and the severity of the response to COVID-19, it has been suggested that treatment with insulin sensitizer drugs in acute COVID-19 disease could modulate the host response and decrease the duration and severity of symptoms and improve long-term outcomes. However, the first generation thiazolidinedione (TZD) insulin sensitizers, which were thought to exert their effect through direct agonism on the PPARγ nuclear receptors and are approved to treat diabetes, are associated with many side effects which would render them impractical or even deleterious in the setting of COVID-19 disease. For these reasons, an international panel of diabetes experts has excluded pioglitazone from a list of medications recommended to treat people with diabetes who are hospitalized with acute COVID-19 disease. In the absence of any other oral treatment, this panel recommended liberal use of insulin to maintain glycemic control in the hospital setting. In contrast, an orally administered safe insulin sensitizer would be an ideal intervention to minimize insulin resistance which dually drives the underlying pathophysiology of COVID-19 disease and is worsened as a result of SARS-CoV-2 infection and might ultimately improve outcomes in patients with diabetes. Moreover, when assessing the potential causes of worse outcomes in people with diabetes or fatty liver disease several key issues stand out including insulin resistance, alteration of pancreatic function, changes in immune function including the cytokine storm and reductions in lymphocytes, downstream vascular pathophysiology, and hepatic damage demonstrated by increases in AST. These issues could be mitigated by an intervention which safely and effectively addresses insulin resistance and dysfunctional adipose function.

The discovery of the mitochondrial pyruvate carrier provided an alternate approach to a new class of insulin sensitizers that delivers the insulin sensitizing pharmacology without the limitations of the direct PPARγ agonists. These findings led to the development of the new insulin sensitizer MSDC-0602K. In a recent 1-year Phase 2b dose-ranging clinical trial in people with fatty liver disease with or without type 2 diabetes (T2D), treatment with MSDC-0602K reduced insulin resistance and improved glycemic control without precipitation of edema (27,28), a key adverse effect that limits the use of PPARγ agonists such as pioglitazone. As discussed below, effective and safe insulin sensitization with MSDC-0602K, could impact what appear to be the drivers of the fulminant pathology in high risk cardiometabolic patients including the abnormal inflammatory reaction and its deadly downstream pathophysiology in response to the SARS-CoV-2 infection. Furthermore, this once-daily oral treatment can be self-administered and could reduce the need for escalation of care.

Given the effects on the metabolic and inflammatory pathways as well as its safety profile, MSDC-0602K could reduce the progression, severity, and duration of COVID-19 disease in individuals with SARS-CoV-2 infection. Reducing the metabolic risk of these individuals early in the course of the infection could limit the extent of the damage and facilitate clearance of the virus. This is a Phase 2 randomized, controlled, double-blind study to evaluate the efficacy and safety of MSDC-0602K in people with SARS-CoV-2 infection. This study enrolls people with known metabolic dysfunction to evaluate the ability of MSC-0602K to mitigate the metabolic dysfunction and disease course of COVID-19.

This study provides critical data regarding the feasibility, safety, and effect size of an insulin sensitization approach, with MSDC-0602K in particular, in the treatment algorithm for people infected with SARS-CoV-2. As the COVID-19 disease pandemic is progressing unabated around the world, it is of utmost importance to study all possible approaches to prevent and treat this disease. There are several additional desirable aspects to this treatment approach: (1) it could readily be used in combination with other proven interventions against COVID-19, like antiviral treatments, with a potential for the two complementary approaches to have an additive beneficial effect on COVID-19 disease course, (2) this intervention could have lasting effects on the metabolic disease course regardless of its effect on COVID-19 disease, thus potentially improving both metabolic as well as COVID-19-related long-term outcomes.

The primary objective of the study is to evaluate the potential of MSDC-0602K treatment to reduce metabolic dysfunction and severity of response to the COVID-19 disease. Secondary objectives include evaluation of specific aspects of the disease state measured by blood samples that are expected to be impacted by the insulin sensitizing pharmacology and which may predict outcomes. Furthermore, secondary objectives include the evaluation of a variety of clinically relevant disease severity markers.

Study Endpoints

Efficacy

Primary Efficacy Endpoints

The primary efficacy endpoint is to evaluate the effects of MSDC-0602K on metabolic dysfunction as indicated by circulating adiponectin in patients recently infected with SARS-CoV-2 and to determine the potential to reduce the absolute score on the COVID-19 severity scale at 14 days post-randomization.

Secondary Efficacy Endpoints

The secondary efficacy endpoints are:
Absolute score on the COVID-19 severity scale at 7 days post-randomization
Absolute score on the COVID-19 severity scale at 21 days post-randomization
Absolute score on the COVID-19 severity scale at 28 days post-randomization
Time to first ER visit for COVID-19-related symptoms through end of study
Time to first hospital admission (any stay of >24 hrs in an acute care facility) for COVID-19-related symptoms through end of study
Hospital length of stay
Time to first ICU admission for COVID-19-related symptoms through end of study
ICU length of stay
Mortality during the study period
Change from baseline to end-of-treatment in inflammatory markers
Change from baseline to end-of-treatment in D-dimer, PAI-1
Change from baseline to end-of-treatment in Differential blood cell counts
Change from baseline to end-of-treatment in insulin resistance (HOMA-IR based on fasting plasma glucose and insulin)
Change from baseline to end-of-treatment in HbA1c
Proportion of subjects needing to intensify glucose control pharmacotherapy
Time to first initiation of any respiratory support
Total days requiring any respiratory support during the study period
Time to resolution of all symptoms (defined as 24 hrs free of symptoms)
Proportion of subjects with any symptoms at 7 days
Proportion of subjects with any symptoms at 14 days
Proportion of subjects with any symptoms at 21 days
Proportion of subjects with any symptoms at 28 days
Proportion of subjects with any symptoms at end of trial

Exploratory Endpoints

The exploratory endpoints are:
Absolute score on the COVID-19 severity scale at end of trial
Exploratory biomarkers to detail effects on improvement in metabolic function

Safety

Safety will be assessed by periodic assessments of vital signs, physical examinations, laboratory parameters, and AEs.

Subject Selection

Approximately 330 subjects with proven SARS-CoV-2 by RT-PCR will be randomized at approximately 1 site in the US. Subjects who do not complete Treatment Period will not be replaced.

Inclusion Criteria

Subjects must meet all the following inclusion criteria to be eligible for enrolment:
1. Willing and able to sign an informed consent document indicating understanding of the purpose of and procedures required for the study and willingness to participate in the study
2. Within 48 hours of a positive SARS-CoV-2 PCR test at the time of screening
3. Age≥40 years
4. BMI≥30 kg/m²
5. Subjects with reproductive potential agree to either practice abstinence or comply with approved double barrier contraceptive method (e.g., condom plus intrauterine device [IUD], condom plus hormonal contraception, or double barrier method, i.e., condoms and diaphragms with spermicidal gel or foam) during Screening and for the duration of the trial.
   Subjects are considered of non-reproductive potential if:
      a. Post-menopausal female with ≥12 consecutive months of spontaneous amenorrhea and age≥55 years at Screening
      b. Surgically sterile female and at least 6 weeks post-sterilization (i.e., bilateral oophorectomy or hysterectomy
      c. Sterilized male at least 1-year post vasectomy
6. At least one additional comorbidity previously diagnosed:
   a. type 2 diabetes (either previously diagnosed or documented HbA1c>6.5% available in the medical record attributable to Type 1 diabetes)
   b. hypertension
   c. atherosclerotic cardiovascular disease
   d. NAFLD/NASH
   e. CKD stage 3 (eGFR 30-60 mL/min/1.73 m²) as demonstrated by the average of the most recent three outpatient readings spanning over at least 2 months

Exclusion Criteria

Subjects who meet any of the following exclusion criteria are not eligible for enrollment:
1. Current eGFR<30 mL/min/1.73 m2 calculated using the CKD-EPI formula based on the last creatinine value in the medical record within 90 days prior to randomization, or a point of care lab value if a qualifying value is not already available
2. HbA1c>11% based on the last value in the medical record within 90 days prior to randomization, or a point of care lab value if a qualifying value is not already available
3. Concomitant use of other agents metabolized through CYP2C8 (i.e., paclitaxel, repaglinide, etc.)
4. Other medical or psychiatric conditions which in the opinion of the investigator renders participation in the study unsafe
5. Previously diagnosed Type 1 diabetes
6. Use of PPAR gamma agonists pioglitazone or rosiglitazone within the 30 days to randomization
7. COVID-19 disease symptoms for >7 days at the time of the screening
8. Current or impending hospitalization at the time of randomization
9. Currently pregnant or intent to become pregnant during the study period, or current breastfeeding
10. Participation in an investigational study (other than a non-treatment registry study) or receipt of an investigational drug within 30 days or 5 half-lives (whichever is longer)

Study Design

Summary of Study Design

This is a randomized, double-blind study of MSDC-0602K (250 mg) or placebo given orally once daily to high risk subjects with a recently identified SARS-CoV-2 infection. Randomization will be stratified by age (≥65 years, Y/N), presence of symptoms at Randomization (Y/N), and history of type 2 diabetes (Y/N).

The primary objective of the study is to evaluate the potential to reduce severity of response to the COVID-19 disease.

Secondary objectives include aspects of the disease state measured by blood samples that are expected to be impacted by the insulin sensitizing pharmacology and which may predict outcomes.

Subjects will undergo a maximum 3-day Screening Period, a Treatment Period of 4 weeks, and a 1-week Follow-up Period. Upon qualification, subjects will be randomized in a 1:1 ratio to once daily administration of either MSDC-0602K (250 mg) or matching placebo, to be taken during the Treatment Period. Following randomization, study subjects will be followed weekly either via phone or in-person: phone contact at Week 1, Week 3, and Week 5; in-person at Randomization & Week 2.

Schedule of Activities

The schedule of activities for the study is shown in Table 8:

The schedule of activities for the study is shown in Table 8

| Study Period | Screening | Treatment | | | | | Follow-up |
|---|---|---|---|---|---|---|---|
| Visit Number | V1¹ | V2 | V3 | V4 | V5 | V6/EOT | V7 |
| Study Week | | Randomization | 1 | 2 | 3 | 4 | 5 |
| Study Day | | 1 | 7 | 14 | 21 | 28 | 35 |
| Window | −3 to 0 | N/A | +/−3 days | | | +/−4 days | +7 days |
| Visit Type | Virtual | In-person | Virtual | In-person | Virtual | In-person | Virtual |
| General Activities | | | | | | | |
| Informed Consent² | X | | | | | | |
| Eligibility Criteria | X | X | | | | | |
| Review | | | | | | | |
| Medical Record | X | | | | | | X |
| Review | | | | | | | |
| Medical | X | X | | | | | |
| History/Medication | | | | | | | |
| History | | | | | | | |
| Physical Exam³ | | X | | X | | X | |
| Vital Signs⁴ | | X | | X | | X | |
| 12-lead ECG | | X | | X | | X | |
| Symptom | X | X | X | X | X | X | X |
| questionnaire | | | | | | | |
| including duration | | | | | | | |
| Modified WHO | | X | X | X | X | X | X |
| COVID severity | | | | | | | |
| scale | | | | | | | |
| Randomization | | X | | | | | |
| Adverse Event | | X | X | X | X | X | X |
| review | | | | | | | |
| Concomitant | | X | X | X | X | X | X |
| Medication review | | | | | | | |
| Study drug | | X | | | | | |
| dispensing | | | | | | | |
| Study drug | | | | X | | X | |
| accountability and | | | | | | | |
| compliance | | | | | | | |
| Point-of-Care | | | | | | | |
| Evaluations | | | | | | | |
| HbA1c (optional)⁵ | | X | | | | | |
| eGFR (optional)⁵ | | X | | | | | |
| Urine Pregnancy | | X | | X | | X | |
| test (FOCP only) | | | | | | | |
| Central Lab | | | | | | | |
| Evaluations | | | | | | | |
| Hematology & | | X | | X | | X | |
| serum chemistry⁵ | | | | | | | |
| Biomarkers | | X | | X | | X | |

¹If subject cannot consent electronically, V1 and V2 may be combined into one visit and performed in-person.
²ICF must be obtained prior to conduct of any study-specific procedures.
³Height is collected at Randomization only; body weight, BMI, and waist circumference measured midway between the lowest rib and the iliac crest are collected.
⁴Vital sign measurements include seated BP, pulse, body temperature, and pulse oxygen.
⁵HbA1c or eGFR will be performed using a point of care device at V2 prior to randomization if documentation of a qualifying result is not available in the medical record within the 90 days prior to randomization.
⁶Minimum 8-hour fasting blood sample.

Method of Assigning Subjects to Treatment

Subjects who meet all the inclusion criteria and none of the exclusion criteria will be allocated centrally by an Interactive Web Response System (IWRS) in a 1:1 allocation ratio to once-daily treatment with either MSDC-0602K 250 mg or placebo, according to a central randomization scheme. Randomization will be stratified by the following:
Age (≥65 years, Y/N)
Presence of symptoms at Randomization (Y/N)
History of type 2 diabetes (Y/N)

Each enrolled subject will be sequentially assigned the next available randomization number within the appropriate randomization stratum and allotted the specified treatment corresponding to that randomization number.

Study Visit 1, Screening Visit (Day −3 to 0)

Subjects with a positive PCR test and who appear to meet study requirements based on available information in the medical record will be contacted by phone to inform them of the study opportunity. If they are interested in participation, the informed consent process will be completed by phone using an electronic informed consent and electronic signature.

Prior to conducting any study-specific procedures, e-informed consent must be obtained from the subject. The nature of the study will be fully explained to each subject during the informed consent process and the subject will have the opportunity to ask questions. An informed consent document will then be e-signed by the subject and the person performing the consent and retained by the investigator. An electronic copy of the informed consent form will be given to the subject.

Rescreening is not allowed in this study. After completing the informed consent process, a subject identification (ID) number will be assigned to a potential study subject. This number will be used to identify the subject for the remainder of the study.

Screening evaluations to determine subject eligibility will be conducted within 72 hours prior to randomization in the study (Screening visit). The screening process can be completed on Day 0 (Randomization Visit).

The following information will be obtained after completing the informed consent process:
Eligibility Review
Medical record review
Medical history/Medication history
Symptom questionnaire including duration

Study Visit 2 (Randomization Visit; Day 0)

Subjects will be instructed to report to the clinic for a study visit following a minimum 8-hour fast prior to providing blood samples. Subjects should not have exercised vigorously in the 48 hours preceding the visit.

The following procedures will be performed for all eligible subjects at Study Visit 2:
Eligibility criteria review
Medical history/medication history review
Physical examination including height and weight
Vital signs measurements, including seated blood pressure, pulse, body temperature, and pulse oxygen
12-lead ECG
Symptom questionnaire including duration
Modified WHO COVID severity scale
AE review
Concomitant medication review
HbA1c via a point-of-care device (optional)
eGFR via a point-of-care device (optional)
Pregnancy test (urine, females of child-bearing potential only)
Blood samples for assessment of serum chemistry and hematology laboratory evaluations
Serum and plasma samples for biomarker testing
Study drug dispensation (1 bottle)

If all Inclusion & none of Exclusion criteria are met, the subject will be randomized, and the study drug dispensed (1 bottle). The first dose of the study medication will be administered on site.

Male subjects will be advised not to donate sperm and to inform female partners of their participation in the study and to use adequate contraceptive methods (including a condom or another form of contraception) if engaging in sexual intercourse with a woman who could become pregnant.

Subjects who are randomized will be scheduled to return to the clinic in approximately 2 weeks for Visit 4. Subjects should be instructed to return unused study medication at the next visit to check compliance and accountability.

Study Visits 3 and 5 (Weeks 1 and 3)

A virtual visit will be conducted at weeks 1 and weeks 3 which consists of:
Symptom questionnaire including duration
Modified WHO COVID severity scale
AE review
Concomitant medication review

Study Visits 4 and 6/EOT (Weeks 2 and 4)

Subjects will be instructed to report to the clinic for a study visit following a minimum 8-hour fast prior to providing blood samples and without having taken their daily dose of study drug. Subjects should not have exercised vigorously in the 48 hours preceding the visit. The following procedures will be performed for all randomized subjects at Visits 4 and 6/EOT:
Physical examination including weight
Vital signs measurements, including seated blood pressure, pulse, body temperature, and pulse oxygen
12-lead ECG
Symptom questionnaire including duration
Modified WHO COVID severity scale
AE review
Concomitant medication review
Pregnancy test (urine, females of child-bearing potential only)
Blood samples for assessment of serum chemistry and hematology laboratory evaluations
Serum and plasma samples for biomarker testing
Study drug accountability and compliance
Study drug return (Study Visit 6/EOT)

Study Visit 7 (Follow-Up Visit)

A virtual visit will be conducted 7 days after Visit 6 (end of treatment) which consists of:
Medical record review
Symptom questionnaire including duration
Modified WHO COVID severity scale
AE review
Concomitant medication review

Discontinuation of Study Drug and Study Termination

Subjects are free to discontinue participation in the study at any time and for any reason without prejudice to future medical care; however, prior to randomization, it should be made clear to potential subjects that early withdrawal from the study including loss to follow-up can be detrimental to the scientific research. Once a subject has been randomized, the investigator will make every reasonable effort to keep the subject in the study. Subjects will continue to be followed to the fullest extent possible after discontinuation of study drug, and all data will be collected regardless of study drug discontinuation, including laboratory assessments.

Subjects may withdraw from study for the following reasons:
Withdrawal of informed consent: A subject should be considered to have withdrawn consent when the subject no longer wishes to participate in any aspect of the study, and does not want any further assessments, visits, or contact. A subject's refusal to participate in specific aspects of the study, such as a refusal to continue taking study drug or to provide blood samples, will NOT constitute withdrawal of consent. Investigators should make every effort to facilitate the subject's continued participation in remaining aspects of the study.
Subject is lost to follow-up: A subject will not be considered lost to follow-up until just prior to database lock and after all efforts to contact the subject have been exhausted.

If a subject withdraws from study for any reason above, vital status (dead or alive) may be obtained through public registries as permitted by local regulations.

Subjects who wish to withdraw consent from the study, to the extent possible, will undergo an End of Treatment evaluation equivalent to Visit 6 (End of Treatment) (see Table 1).

The appropriate case report forms (eCRFs) should be completed, including an explanation as to why the subject's participation in the study is being discontinued. Subjects who do not complete the study will not be replaced.

The investigator and Cirius Therapeutics, Inc, have the right to discontinue study drug dosing in any subject in the event of an intercurrent illness, AE, protocol violation, or other reason including, but not limited to:
Need for any treatment not allowed by the protocol
Subject's inability to comply with protocol-specified procedures
Sites with unacceptable subject early termination rates
Sites with serious Good Clinical Practice (GCP) or quality concerns
AE that in the opinion of the investigator requires discontinuation of study drug

Subjects who have discontinued study drug may be considered for resumption of dosing upon consultation with a study medical monitor.

Management of Type 2 Diabetes

Concomitant Glycemic Control Medications

Glycemic control should be managed by the subjects' healthcare provider but use of TZDs (pioglitazone or rosiglitazone) is prohibited during the study. Use of insulin is allowed, if needed, to control blood glucose levels. Care must be taken to avoid hypoglycemia since those on study drug may show a larger response than may be expected.

Management of Liver Function Marker Changes Subjects with New Elevations in Liver Biochemistries from Normal Baseline

COVID-19 infections often present with elevation of liver enzymes. The study drug has been shown to reduce circulating levels of liver enzymes in subjects with NAFLD/NASH with and without diabetes, but it has not been studied in subjects with COVID-19 disease. For new elevations in transaminases greater than 2× the ULN in subjects with normal baseline values or 2× baseline in subjects with abnormal baseline values, repeat measurement should be performed within 48 to 72 hours. If elevations persist, subjects should be evaluated for other causes of transaminase elevations and with tests of hepatic function. If no other cause is found, then subjects need to be “Monitored Closely” (see Section 5.5.3). Study drug should be discontinued, and the subject should be followed until resolution of symptoms or signs, in the following situations:
ALT or AST>8×ULN
ALT or AST>5×ULN for more than 2 weeks
ALT or AST>3×ULN and (total bilirubin>2×ULN or INR>1.5)
ALT or AST>3×ULN with the appearance of fatigue, nausea, vomiting, right upper quadrant pain or tenderness, fever, rash, and/or eosinophilia (>5%)

If a subject lives in a remote area, laboratory testing can be performed locally, and the results should be promptly communicated to the investigator site.

Subjects with Elevated Baseline in Liver Biochemistries

If subjects with abnormal baseline liver indices develop elevations of AST or ALT>2×baseline or total bilirubin>1.5× baseline values during the study, repeat testing should be performed within 48 to 72 hours. If there are persistent elevations (ALT or AST>2× baseline or total bilirubin>1.5× baseline values) upon repeat testing, then close observation (testing and physical or virtual examination) should be implemented and discontinuation of drug should be considered (see Section 5.5.3).

A decision to discontinue or temporarily interrupt the study drug should be considered based on factors that include how much higher than baseline the ALT and AST values are relative to the ULN and how much the on-study ALT and AST levels have increased relative to baseline, in addition to whether there is concomitant elevation of bilirubin or INR. Discontinue or temporarily interrupt study drug if any of the following occurs:
Baseline measurements were <2×ULN and ALT or AST increases to >5× baseline,
Baseline measurements were ≥2×ULN but <5×ULN, and ALT or AST increases to >3× baseline measurement
Baseline measurements were ≥5×ULN, and ALT or AST increases to >2× baseline
ALT or AST increases >2× baseline AND the increase is accompanied by a concomitant increase in total bilirubin to >2× baseline OR the INR concomitantly increases by >0.2
Any signs and symptoms of fatigue, nausea, vomiting, right upper quadrant pain or tenderness, fever, rash, and/or eosinophilia (>5%).

Re-initiation of study drug may be considered after consultation with the sponsor medical monitor.

If a subject lives in a remote area, laboratory testing can be performed locally.

Close Observation for Suspected DILI

Close observation for suspected DILI includes:
1. Repeating liver enzyme and serum bilirubin tests. Frequency of repeat testing can decrease to once a week or less if abnormalities stabilize or the trial drug has been discontinued and the subject is asymptomatic.
2. Obtaining a more detailed history of symptoms and prior or concurrent diseases.
3. Obtaining a history of concomitant drug use (including nonprescription medications and herbal and dietary supplement preparations), alcohol use, recreational drug use, and special diets.
4. Ruling out acute viral hepatitis types A, B, C, D, and E; autoimmune or alcoholic hepatitis; hypoxic/ischemic hepatopathy; and biliary tract disease.
5. Obtaining a history of exposure to environmental chemical agents.
6. Obtaining additional tests to evaluate liver function, as appropriate (e.g., INR, direct bilirubin).
7. Considering gastroenterology or hepatology consultations.

If a new concomitant medication with known hepatotoxicity is started and liver function tests rise to meet the above criteria, the known possible hepatotoxin should be discontinued (replaced) if possible and liver function tests followed in the same manner as outlined above. If liver function tests remain elevated or increase further, then the study drug should be discontinued (and subjects should continue with the study visits).

Study Procedures and Evaluations

Vital Signs

Vital signs will be measured at Randomization, Week 2 and Week 6/EOT. Vital sign measurements will include blood pressure, pulse, body temperature, pulse oxygen. Blood pressure should be measured after the subject has rested for 10 minutes in a seated position using an appropriately-sized cuff.

Physical Examinations

The physical examination will include signs of congestion/edema, signs of cirrhosis, complete CV, pulmonary, neurologic, and extremities examinations, as well as any other abnormalities of significance. Body weight, BMI and waist circumference will be collected at Randomization, Week 2 and Week 6/EOT; height will be recorded only at Randomization Visit.

Electrocardiogram

A single 12-lead ECG will be performed after the subject has rested quietly for at least 10 minutes in a supine position. The QT correction derived by the ECG machine or computed manually should be recorded. A single repeat ECG may be done at the investigator's discretion.

Point-of-Care Evaluations

A point-of-care device may be used at Visit 2 prior to randomization if documentation of a qualifying result for HbA1c or eGFR is not available in the medical record within the 90 days prior to randomization. A point-of-care pregnancy testing will be used on females of childbearing potential only.

Laboratory Assessments

Instructions for collecting, processing, storing and shipping of blood samples for pharmacologic and pharmacokinetic analyses will be provided in the Laboratory Manual. All blood samples for serum chemistry and hematology should be drawn in a fasted state (minimum 8 hours). A certified central clinical laboratory will be used to perform all routine hematology and serum chemistry assays. The central laboratory reference ranges will be used. Local laboratory assessments may be used to manage subject care during the conduct of the study (e.g., routine care, unscheduled visits, urgent care).

Study Drug

Investigational and Control Drugs

The sponsor will provide the following double-blind study drugs:
MSDC-0602K 250 mg tablets
Matching Placebo tablets

MSDC-0602K tablets and placebo tablets will be packaged into 33 count bottles.

Treatments

At Visit 2, all eligible subjects will be randomized in a 1:1 allocation ratio to one of the 2 treatment arms listed below:
MSDC-0602K 250 mg tablets
Matching Placebo tablets

Subjects will be instructed to take 1 tablet daily by mouth with water at least 30 minutes prior to a meal for the duration of the study. The subject should be instructed to swallow the tablet whole, and not to chew, cut, or crush the tablet.

Randomization Code Creation and Storage

Randomization personnel of the sponsor or designee will generate the randomization schedule. All randomization information will be securely stored, accessible only by authorized personnel.

Study Drug Blinding

The study is double-blind in that neither the subjects nor the investigator will be aware of the treatment administered. Blinding will be maintained throughout the study by use of active and placebo tablets of similar appearance. The series of random numbers from which the randomization algorithm operates will be created by personnel who will have no involvement in the day-to-day operations of the study. The random number series will be sequestered from all blinded study personnel, assuring there will be no unblinding information available to them until the study completes.

Study Drug Assignment and Dispensing Procedures

Study Drug Assignment

At Visit 2 (Randomization Visit), subjects who fulfill all the inclusion/exclusion criteria will be randomized to one of the two treatment arms via the IWRS. Each subject will be uniquely identified in the study by a subject ID number comprised of a combination of his/her site number and subject number. Once assigned to a subject, the subject ID number will not be reused. At Visit 2, one bottle of study drug will be dispensed via the IWRS system A separate User Manual will be provided with details for use of the IWRS.

Subjects, investigator staff, and persons performing the assessments will remain blinded to the identity of the treatment from the time of randomization until database lock, using the following methods:
1. Randomization data are kept strictly confidential until the time of unblinding, and will not be accessible by anyone involved in the day-to-day conduct of the study (except for emergency unblinding as noted in Section 7.9).
2. The identity of the treatments will be concealed by the use of study drugs that are identical in packaging, labeling, and schedule of administration, appearance, taste and odor.

The IWRS will maintain a record that identifies each subject and the treatment/study drug of an individual subject's treatment assignment. Unblinding of all subjects' treatment assignments will occur at the conclusion of the trial following final database lock.

Dispensing the Study Drug

Study site will be supplied by sponsor or designee with study drug in identically appearing packaging. The clinical supplies will be packaged and labeled in a double-blind fashion. Each bottle will contain 33 tablets. One bottle will be dispensed to subjects at Visit 2. Instructions will be provided to advise the site on the proper method of randomizing the subject in the IWRS and selecting the correct medication ID numbered bottle for dispensing to a given subject at Visit 2. Subjects will be instructed to self-administer study drug once daily with water at least 30 minutes before a meal. They will be instructed to swallow the drug whole and not to chew, cut, or crush the tablet.

Study Drug Interruptions

Treatment interruptions (3 or more consecutive doses) should be avoided. A drug interruption not due to a safety concern occurring at any time during any period will be considered a protocol deviation. An occasional missed tablet is not considered treatment interruption. All changes will be recorded in the study records.

Compliance

Treatment compliance will be assessed by monitoring drug accountability. The subject will be asked to return all used, partly used, and unused study medication bottles at Visit 4 and Visit 6/EOT. The investigator will compare the amount of study drug returned with the amount dispensed and question the subject in case of discrepancies. If warranted, the investigator will remind the subject of the importance of taking the study medication as prescribed. These discussions should be documented in the subject's medical record.

Study Drug Supply, Storage, and Tracking

Study drugs must be received by a designated person at the study site, handled and stored safely and properly, and kept in a secured location to which only the investigator and designated site staff have access. Upon receipt, all study drugs should be stored according to the instructions specified on the drug labels. Study drug must be kept in a secure cabinet or room with access restricted to only necessary study site personnel until it is used. Unused medication may be destroyed onsite with sponsor's approval or returned to the sponsor or designee for destruction. Clinical supplies are to be dispensed only in accordance with the protocol.

Medication labels will comply with the legal requirements of the countries where the study is being conducted. They will include storage conditions for the drug, but no information about the subject. The investigator must maintain an accurate record of the receipt of shipments and dispensing of study drug in a drug accountability form. Monitoring of drug accountability will be performed by the monitor during site visits and at the completion of the trial.

At the conclusion of the study, and as appropriate during the course of the study, the investigator will dispose or return all used and unused study drug to the sponsor or designee only after accountability is performed by the study monitor.

Emergency Unblinding

Unblinding is restricted to emergency situations and should be used only under circumstances where knowledge of the treatment is necessary for the proper management of the subject. When possible, the investigator should attempt to contact the medical monitor, sponsor, or designee before unblinding a subject's treatment assignment. The randomization code can only be broken if an emergency situation arises that, in the investigator's opinion, requires knowledge of the drug product dosed for management of the emergency medical condition. To unblind a subject's treatment assignment, the investigator will access the unblinding module within the IWRS. Instructions for breaking the blind will be provided to the site. Unless discontinued for a safety reason, study drug may be continued following unblinding of the subject's treatment assignment.

Analysis of Key Efficacy Endpoints

Primary Efficacy Endpoints

Adiponectin

Low levels of adiponectin are indicative of inflamed, dysfunctional adipose tissue and treatment with MSDC-0602 should increase adiponectin levels during the time frame of this trial. The mean change from baseline endpoints will be analyzed using an Analysis of Covariance (ANCOVA) model and will summarized using mean, median, standard deviations, 25th and 75th percentiles, min, and max.

COVID-19 Severity

The primary endpoint of disease severity will use the modified WHO COVID-19 ordinal scale (range 0 to 8, with higher ranks representing higher severity) as measured on visit 4 (approximately 14 days) The primary endpoint of COVID-19 severity will be analyzed using an ordinal logistic regression model assuming proportional odds and adjusting for treatment, sex, presence of diabetes mellitus, BMI, and age. Analysis will be according to the treatment groups as randomized. The odds ratio and the corresponding 95% confidence interval and p-values will be provided. A secondary analysis of the primary endpoint will use a binary analysis approach (chi-square test for homogeneity of proportions), for severity scores >1 or <1.

Key Secondary Efficacy Endpoints

The secondary efficacy endpoints related to the COVID-19 severity score will also be analyzed using the same logistic regression models and adjusting for similar covariates as used for the primary endpoint. The odds ratio and the corresponding 95% confidence interval and p-values will be provided. A secondary analysis of these endpoints will also be conducted using a binary analysis approach (chi-square test for homogeneity of proportions). For continuous variables, the mean change from baseline endpoints will be analyzed using an Analysis of Covariance (ANCOVA) model. Continuous variables will be summarized using mean, median, standard deviations, 25th and 75th percentiles, min, and max. Categorical variables will be summarized using number and percentages. All analyses will be on the intent-to-treat population. All statistical tests and confidence intervals will be two-sided. These measurements will provide information for construction of post-hoc hypotheses about which clinical presentations (e.g., inflammatory markers, cell counts, markers of hemostasis) might be related to outcomes.

### Example 8. Reducing Sequalae of COVID-19 Disease Severity by Targeting Metabolic Dysfunction with MSDC-0602K

The study is studied to determine the consequences of poor outcomes to SARS-CoV2 infection could be addressed by MSDC-0602K. Multiple organ systems are involved in ongoing issues 4-12 weeks after acute COVID-19. Persistent symptoms are not attributable to other diagnoses. Further, worsening insulin resistance can worsen diabetes or lead to new onset diabetes.

This is a randomized, double-blind study of MSDC-0602K (250 mg) or placebo given to subjects at high-risk for adverse outcomes from a recently identified symptomatic SARS-CoV-2 infection.

The primary objective of the study is to evaluate the potential of treatment with MSDC-0602K to reduce metabolic dysfunction as measured by the increase in plasma adiponectin and to estimate the potential of this treatment to reduce the severity of response to the COVID-19 disease Secondary objectives include aspects of the disease state measured by MRI and by blood samples, which are both expected to be impacted by the insulin sensitizing pharmacology and which might also predict recovery from COVID-19.

Subjects undergo a Screening Period of up to 4 days. At the end of the Screening period, qualified subjects are randomized in a 1:1 ratio to either MSDC-0602K (250 mg) or matching placebo to be taken by mouth once daily during the 4-week Treatment Period of the study. There are 3 visits to the clinic followed by a virtual follow-up after approximately 2 weeks.

Study Objectives

The primary objective of the study is to evaluate the potential of MSDC-0602K treatment to reduce metabolic dysfunction and long-term sequela COVID-19 disease.

Secondary objectives include evaluation of specific aspects of the disease state measured by blood samples and MRI imaging of the liver and the pancreas. These parameters are expected to be impacted by the insulin sensitizing pharmacology and may predict outcomes. Furthermore, this study provides for specific outcome hypotheses to be powered for in a confirmatory trial(s).

Study Endpoints

Primary Efficacy Endpoints: Effect of MSDC-0602K treatment to reduce adipose inflammation based on circulating adiponectin and estimation of the potential to reduce COVID-19 long term sequelae.

The secondary efficacy endpoints are:
Any requirement for hospital care during the 3-month study
Clinical/Patient Reported Outcomes
Change from V1 to V4 in insulin, C-peptide, fasting glucose versus placebo
Change from V1 to V4 in AST, ALT, ALP, GTT versus placebo
Change from V1 to V4 in CCL2 and cytokine panel versus placebo
Change from V1 to V4 in total cell and platelet counts versus placebo
Change from V1 to V4 in d-dimer and PAI-1 versus placebo
HbA1c change from V1 to V4 versus placebo
Addition of other medications (including insulin)
Proportion of subjects with any symptoms at visit 4 endpoint

The exploratory endpoints are:
Transcriptional analysis of circulating PBMCs by flow cytometry and sequencing
Various post-hoc analyses of the potential relationship of biomarkers, ectopic fat in the liver and pancreas, and clinical progression based on composite analyses
Samples for exploratory biomarkers

Approximately 220 subjects with proven SARS-CoV-2 by RT-PCR will be randomized at approximately 2-4 sites in the US. Subjects who do not complete Treatment Period will not be replaced.

The inclusion criteria for subjects are listed below:
Willing and able to sign an informed consent document indicating understanding of the purpose of and procedures required for the study and willingness to participate in the study
Within 48 hours of a positive SARS-CoV-2 PCR test at the time of screening
Present with symptoms consistent with active COVID-19
Willing and able to undergo MRI at two visit
Age≥40 years
BMI≥30 kg/m²
Subjects with reproductive potential agree to either practice abstinence or comply with approved double barrier contraceptive method (e.g., condom plus intrauterine device [IUD], condom plus hormonal contraception, or double barrier method, i.e., condoms and diaphragms with spermicidal gel or foam) during Screening and for the duration of the trial.
Subjects are considered of non-reproductive potential if:
   a. Post-menopausal female with ≥12 consecutive months of spontaneous amenorrhea and age≥55 years at Screening
   b. Surgically sterile female and at least 6 weeks post-sterilization (i.e., bilateral oophorectomy or hysterectomy
   c. Sterilized male at least 1-year post vasectomy
At least one additional comorbidity previously diagnosed:
   a. type 2 diabetes (either previously diagnosed or documented HbA1c>6.5% available in the medical record attributable to Type 1 diabetes)
   b. hypertension
   c. atherosclerotic cardiovascular disease
   d. NAFLD/NASH
   e. CKD stage 3 (eGFR 30-60 mL/min/1.73 m2) as demonstrated by the average of the most recent three outpatient readings spanning over at least 2 months

The exclusion criteria for subjects are listed below:
Current eGFR<30 mL/min/1.73 m2 calculated using the CKD-EPI formula based on the last creatinine value in the medical record within 90 days prior to randomization, or a point of care lab value if a qualifying value is not already available
HbA1c>11% based on the last value in the medical record within 90 days prior to randomization, or a point of care lab value if a qualifying value is not already available
Concomitant use of other agents metabolized through CYP2C8 (i.e., paclitaxel, repaglinide, etc.)
Other medical or psychiatric conditions which in the opinion of the investigator renders participation in the study unsafe
Previously diagnosed Type 1 diabetes
Use of PPAR gamma agonists pioglitazone or rosiglitazone within the 30 days to randomization
Current or impending hospitalization at the time of randomization
Have been treated for more than one day in an intensive care unit
Currently pregnant or intent to become pregnant during the study period, or current breastfeeding
Participation in an investigational study (other than a non-treatment registry study) or receipt of an investigational drug within 30 days or 5 half-lives (whichever is longer)
Primary: Adiponectin is a measure of insulin sensitivity and reduced adipose inflammation. Any N>30 is 90% powered to show a difference.

Study Design

This is a randomized, double-blind study of MSDC-0602K (250 mg) or placebo given orally once daily to high risk subjects with an active SARS-CoV-2 infection and symptoms. Randomization will be stratified by whether or not the patients have been vaccinated against SARS-CoV2 (previous vaccination Y/N) and whether or not there is a history of type 2 diabetes (Y/N).

The primary objective of the study is to evaluate the potential to reduce metabolic inflammation and improve recovery from the COVID-19 disease.

Secondary objectives include aspects of the disease state measured by blood samples and MRI imaging of the pancreas and the liver, which are expected to be impacted by the insulin sensitizing pharmacology. We hypothesize that the improvement produced by the insulin sensitizing pharmacology will also improve the ability of the patient to recover from the infection.

Subjects will undergo a maximum 4-day Screening Period, a Treatment Period of 3 months, and a 2-week Follow-up Period. Upon qualification, subjects will be randomized in a 1:1 ratio to once daily administration of either MSDC-0602K (250 mg) or matching placebo, to be taken during the Treatment Period. Following randomization, study subjects will be followed by visits at 1 week, 1 month, and 3 months. There will be a follow up phone visit 2 weeks after completion of the study.

Schedule of Activities:

| Study Period | Screening¹ | Follow-up | | | | |
|---|---|---|---|---|---|---|
| Visit Number | | V1 | V2 | V3 | V4 | Follow up |
| Study Week | | Randomization | 1 | 4 | 12 | 14 |
| Study Day | | 1 Baseline | 7 | 28 | 84 | 98 |
| | | | | | Endpoint | |
| Window | −4 to 0 | N/A | +/−3 days | | | +3 days |
| Visit Type | Virtual | Clinic | Virtual | Clinic | Clinic | Virtual |
| General Activities | | | | | | |
| Informed Consent² | X | | | | | |
| Eligibility Criteria | X | X | | | | |
| Review | | | | | | |
| Medical Record | X | | | | | X |
| Review | | | | | | |
| Medical | X | X | | | | |
| History/Medication | | | | | | |
| History | | | | | | |
| Physical Exam³ | | X | | X | | |
| Vital Signs⁴ | | X | | X | | |
| 12-lead ECG | | X | | X | | |
| CRO/PRO | | X | X | X | X | |
| Modified WHO | | X | | X | X | X |
| COVID severity | | | | | | |
| scale | | | | | | |
| Randomization | | X | | | | |
| Adverse Event | | X | X | X | X | |
| review | | | | | | |
| Concomitant | | X | X | X | X | |
| Medication review | | | | | | |
| Study drug | | X | | X | | |
| dispensing | | | | | | |
| Study drug | | | | X | X | |
| accountability and | | | | | | |
| compliance | | | | | | |
| Point-of-Care | | | | | | |
| Evaluations | | | | | | |
| HbA1c, eGFR | | X | | | | |
| Pregnancy | | X | | X | X | |
| Central Lab | | | | | | |
| Evaluations | | | | | | |
| Hematology & | | X | | X | X | |
| serum chemistry⁵ | | | | | | |
| Biomarkers | | X | | X | X | |
| Imaging | | | | | | |
| MRI-CoverScan | | X | | | X | |

¹If subject cannot consent electronically, portions of the screening visit and Visit 1 may be combined into one visit and performed in-person.
²ICF must be obtained prior to conduct of any study-specific procedures.
³Height is collected at Randomization only; body weight, BMI, and waist circumference measured midway between the lowest rib and the iliac crest are collected.
⁴Vital sign measurements include seated BP, pulse, body temperature, and pulse oxygen.
⁵HbA1c or eGFR will be performed using a point of care device at V1 prior to randomization if documentation of a qualifying result is not available in the medical record within the 90 days prior to randomization.
⁶Minimum 8-hour fasting blood sample.

Method of Assigning Subjects to Treatment

Subjects who meet all the inclusion criteria and none of the exclusion criteria will be allocated centrally by an Interactive Web Response System (IWRS) in a 1:1 allocation ratio to once-daily treatment with either MSDC-0602K 250 mg or placebo, according to a central randomization scheme. Randomization will be stratified by the following:
Vaccination against SARS CoV2 (Y/N)
History of type 2 diabetes (Y/N)

Each enrolled subject will be sequentially assigned the next available randomization number within the appropriate randomization stratum and allotted the specified treatment corresponding to that randomization number.

Screening Visit (Day −4 to 0)

Subjects with a positive PCR test and who appear to meet study requirements based on available information in the medical record will be contacted by phone to inform them of the study opportunity. If they are interested in participation, the informed consent process will be completed by phone using an electronic informed consent and electronic signature.

Prior to conducting any study-specific procedures, e-informed consent must be obtained from the subject. The nature of the study will be fully explained to each subject during the informed consent process and the subject will have the opportunity to ask questions. An informed consent document will then be e-signed by the subject and the person performing the consent discussion, and retained by the investigator. An electronic copy of the informed consent form will be given to the subject.

Rescreening is not allowed in this study. After completing the informed consent process, a subject identification (ID) number will be manually assigned to a potential study subject. This number will be used to identify the subject for the remainder of the study.

Screening evaluations to determine subject eligibility will be conducted within 72 hours prior to randomization in the study (Screening visit). The screening process can be completed on Day 0 (Randomization Visit).

The following information will be obtained after completing the informed consent process:
Eligibility Review
Medical record review
Medical history/Medication history
Symptom questionnaire including duration

Study Visit 1 (Randomization Visit; Day 0 Baseline)

Subjects will be instructed to report to the clinic for a study visit following a minimum 8-hour fast prior to providing blood samples. Subjects should not have exercised vigorously in the 48 hours preceding the visit.

The following procedures will be performed for all eligible subjects at Study Visit 2:
Eligibility criteria review
Medical history/medication history review
Physical examination including height and weight
Vital signs measurements, including seated blood pressure, pulse, body temperature, and pulse oxygen
12-lead ECG
Symptom questionnaire including duration
CRO/PRO Symptom questionnaires
AE review
Concomitant medication review
HbA1c via a point-of-care device (if needed)
eGFR via a point-of-care device (if needed)
Pregnancy test (urine, females of child-bearing potential only)
Blood samples for assessment of serum chemistry and hematology laboratory evaluations
Serum and plasma samples for biomarker testing and PBMCs
CoverScan MRI
First does given and study drug dispensation (1 bottle)

The final criteria for qualification will be obtained by point of care testing which may be done at any time during the screening period. If all Inclusion & none of Exclusion criteria are met, the subject will be randomized. Collection of baseline samples for central lab analysis baseline MRI may be collected on the same data, or if necessary over the next 2 days, before administration of randomized study drug. Following the last of the baseline measurements, the study drug dispensed (1 bottle with 33 pills). The first dose of the study medication will be administered on site and the subject will be given the study drug to take home.

Male subjects will be advised not to donate sperm and to inform female partners of their participation in the study and to use adequate contraceptive methods (including a condom or another form of contraception) if engaging in sexual intercourse with a woman who could become pregnant.

Subjects are advised to contact the study coordinator immediately if their symptoms worsen. Treatment of all subjects should follow current standard of care including whatever escalation of treatment is considered necessary and appropriate. In such cases the study, continuance of the study protocol will be up to the attending medical care and the Subjects who are randomized will be scheduled to return to the clinic in approximately 1 week for Visit 2.

Study Visit 2 (Weeks 1)

This is a virtual report by phone at day 7 (+/−3 days)
Symptom questionnaires
AE review
Concomitant medication review

Subjects will be scheduled for the one month for study visit 3 and reminded to show up to the clinic fasted and without taking their morning study medication to bring residual study drug supplies.

Study Visits 3 (Week 4)

Subjects will be instructed to report to the clinic for a study visit following a minimum 8-hour fast prior to providing blood samples and without having taken their daily dose of study drug. Subjects should not have exercised vigorously in the 48 hours preceding the visit. The following procedures will be performed for all randomized subjects at Visit 3:
CRO/PRO Symptom questionnaires
Limited physical examination
AE review
Vital signs measurements, including seated blood pressure, pulse, body temperature, and pulse oxygen
Concomitant medication review
Pregnancy test (urine, females of child-bearing potential only)
Blood samples for assessment of serum chemistry and hematology laboratory evaluations
Serum and plasma samples for biomarker testing and PBMCs
Study drug accountability and compliance
Dispense two bottles of study drug (33 count each) and schedule 12-week endpoint visit.

Study Visit 4 (12-Week Endpoint)

Subjects will be instructed to report to the clinic for a study visit following a minimum 8-hour fast prior to providing blood samples and without having taken their daily dose of study drug. Subjects should not have exercised vigorously in the 48 hours preceding the visit. The following procedures will be performed for all randomized subjects at Visit 4.
CRO/PRO Symptom questionnaires
Limited physical examination
Vital signs measurements, including seated blood pressure, pulse, body temperature, and pulse oxygen
AE review
Concomitant medication review
Pregnancy test (urine, females of child-bearing potential only)
Blood samples for assessment of serum chemistry and hematology laboratory evaluations
Serum and plasma samples for biomarker testing
Study drug accountability and compliance
CoverScan MRI

Follow Up Phone Call

Approximately 2 weeks after the end point visit the subjects will be contacted by phone to record any notable events since the end point visit.

Study drug accountability and compliance Discontinuation of Study Drug and Study Termination

Subjects are free to discontinue participation in the study at any time and for any reason without prejudice to future medical care; however, prior to randomization, it should be made clear to potential subjects that early withdrawal from the study including loss to follow-up can be extremely damaging to the scientific research. Once a subject has been randomized, the investigator will make every reasonable effort to keep the subject in the study. Subjects will continue to be followed to the fullest extent possible after discontinuation of study drug, and all data will be collected regardless of study drug discontinuation, including laboratory assessments.

Subjects may withdraw from study for the following reasons:
Withdrawal of informed consent: A subject should be considered to have withdrawn consent when the subject no longer wishes to participate in any aspect of the study, and does not want any further assessments, visits, or contact. A subject's refusal to participate in specific aspects of the study, such as a refusal to continue taking study drug or to provide blood samples, will NOT constitute withdrawal of consent. Investigators should make every effort to facilitate the subject's continued participation in remaining aspects of the study.
Subject is lost to follow-up: A subject will not be considered lost to follow-up until just prior to database lock and after all efforts to contact the subject have been exhausted.

If a subject withdraws from study for any reason above, vital status (dead or alive) may be obtained through public registries as permitted by local regulations.

Subjects who wish to withdraw consent from the study, to the extent possible, will undergo an End of Treatment evaluation equivalent to Visit 6 (End of Treatment) (see Table 1).

The appropriate case report forms (eCRFs) should be completed, including an explanation as to why the subject's participation in the study is being discontinued. Subjects who do not complete the study will not be replaced.

The investigator and Cirius Therapeutics, Inc, have the right to discontinue study drug dosing in any subject in the event of an intercurrent illness, AE, protocol violation, or other reason including, but not limited to:
Need for any treatment not allowed by the protocol
Subject's inability to comply with protocol-specified procedures
Sites with unacceptable subject early termination rates
Sites with serious g Good Clinical Practice (GCP) or quality concerns
AE that in the opinion of the investigator requires discontinuation of study drug

Subjects who have discontinued study drug may be considered for resumption of dosing upon consultation with a study medical monitor.

Management of Liver Function Marker Changes

Subjects with New Elevations in Liver Biochemistries from Normal Baseline

COVID-19 infections often present with elevation of liver enzymes. The study drug has been shown to reduce circulating levels of liver enzymes in subjects with NAFLD/NASH with and without diabetes, but it has not been studied in subjects with COVID-19 disease. For new elevations in transaminases greater than 2× the ULN in subjects with normal baseline values or 2×baseline in subjects with abnormal baseline values, repeat measurement should be performed within 48 to 72 hours. If elevations persist, subjects should be evaluated for other causes of transaminase elevations and with tests of hepatic function. If no other cause is found, then subjects need to be “Monitored Closely” (see Section 5.5.3). Study drug should be discontinued, and the subject should be followed until resolution of symptoms or signs, in the following situations:
ALT or AST>8×ULN
ALT or AST>5×ULN for more than 2 weeks
ALT or AST>3×ULN and (total bilirubin>2×ULN or INR>1.5)
ALT or AST>3×ULN with the appearance of fatigue, nausea, vomiting, right upper quadrant pain or tenderness, fever, rash, and/or eosinophilia (>5%)

If a subject lives in a remote area, laboratory testing can be performed locally, and the results should be promptly communicated to the investigator site.

Subjects with Elevated Baseline in Liver Biochemistries

If subjects with abnormal baseline liver indices develop elevations of AST or ALT>2×baseline or total bilirubin>1.5×baseline values during the study, repeat testing should be performed within 48 to 72 hours. If there are persistent elevations (ALT or AST>2×baseline or total bilirubin>1.5×baseline values) upon repeat testing, then close observation (testing and physical examination 2-3 times per week) should be implemented and discontinuation of drug should be considered (see Section 5.5.3).

A decision to discontinue or temporarily interrupt the study drug should be considered based on factors that include how much higher than baseline the ALT and AST values are relative to the ULN and how much the on-study ALT and AST levels have increased relative to baseline, in addition to whether there is concomitant elevation of bilirubin or INR. Discontinue or temporarily interrupt study drug if any of the following occurs:
Baseline measurements were <2×ULN and ALT or AST increases to >5×baseline,
Baseline measurements were ≥2×ULN but <5×ULN, and ALT or AST increases to >3×baseline measurement
Baseline measurements were ≥5×ULN, and ALT or AST increases to >2×baseline
ALT or AST increases >2×baseline AND the increase is accompanied by a concomitant increase in total bilirubin to >2×baseline OR the INR concomitantly increases by >0.2
Any signs and symptoms of fatigue, nausea, vomiting, right upper quadrant pain or tenderness, fever, rash, and/or eosinophilia (>5%).

Re-initiation of study drug may be considered after consultation with the sponsor medical monitor.

If a subject lives in a remote area, laboratory testing can be performed locally.

Close Observation for Suspected DILI

Close observation for suspected DILI includes:
Repeating liver enzyme and serum bilirubin tests. Frequency of repeat testing can decrease to once a week or less if abnormalities stabilize or the trial drug has been discontinued and the subject is asymptomatic.
Obtaining a more detailed history of symptoms and prior or concurrent diseases.
Obtaining a history of concomitant drug use (including nonprescription medications and herbal and dietary supplement preparations), alcohol use, recreational drug use, and special diets.
Ruling out acute viral hepatitis types A, B, C, D, and E; autoimmune or alcoholic hepatitis; hypoxic/ischemic hepatopathy; and biliary tract disease.
Obtaining a history of exposure to environmental chemical agents.
Obtaining additional tests to evaluate liver function, as appropriate (e.g., INR, direct bilirubin).
Considering gastroenterology or hepatology consultations.

If a new concomitant medication with known hepatotoxicity is started and liver function tests rise to meet the above criteria, the known possible hepatotoxin should be discontinued (replaced) if possible and liver function tests followed in the same manner as outlined above. If liver function tests remain elevated or increase further, then the study drug should be discontinued (and subjects should continue with the study visits).

Study Procedures and Evaluations

Vital Signs

Vital signs will be measured at Randomization and treatment visits. Vital sign measurements will include blood pressure, pulse, body temperature, pulse oxygen. Blood pressure should be measured after the subject has rested for 10 minutes in a seated position using an appropriately-sized cuff

Physical Examinations

The physical examination will include signs of congestion/edema, signs of cirrhosis, complete CV, pulmonary, neurologic, and extremities examinations, as well as any other abnormalities of significance. Body weight, BMI and waist circumference will be collected at Randomization, and all treatment visits; height will be recorded only at Randomization Visit.

Electrocardiogram

A single 12-lead ECG will be performed after the subject has rested quietly for at least 10 minutes in a supine position. The QT correction derived by the ECG machine or computed manually should be recorded. A single repeat ECG may be done at the investigator's discretion.

Point-of-Care Evaluations

A point-of-care device may be used at Visit 1 prior to randomization if documentation of a qualifying result for HbA1c or eGFR is not available in the medical record within the 90 days prior to randomization. A point-of-care pregnancy testing will be used on females of childbearing potential only.

Laboratory Assessments

Instructions for collecting, processing, storing and shipping of blood samples for pharmacologic and pharmacokinetic analyses will be provided in the Laboratory Manual.

All blood samples for serum chemistry and hematology should be drawn in a fasted state (minimum 8 hours). A certified central clinical laboratory will be used to perform all routine hematology and serum chemistry assays.

The central laboratory reference ranges will be used. Local laboratory assessments may be used to manage subject care during the conduct of the study (e.g., routine care, unscheduled visits, urgent care).

TABLE 9
provides a summary of laboratory tests performed during the study.

| Hematology | Serum/Plasma Chemistry | Other Biomarkers |
|---|---|---|
| Hemoglobin | Albumin | Cytokines (hsCRP, IL6, |
| Hematocrit | Alkaline phosphatase | CCL2, TNFa) |
| Platelets | Alanine aminotransferase (ALT) | D-dimer |
| Red blood cells | Aspartate aminotransferase (AST) | PAI-1 |
| (RBCs) with indices | Blood urea nitrogen (BUN) | HMW adiponectin |
| White blood cells | Calcium | Samples for PBMCs |
| (WBCs) with | Carbon dioxide | Sored samples for |
| differential | Chloride | additional exploratory |
| Glycosylated | Creatinine | biomarkers |
| hemoglobin | Fasting plasma glucose (FPG) | |
| (HbA1c) | Fasting insulin | |
| | Lactate dehydrogenase (LDH) | |
| | Ferritin | |
| | Potassium | |
| | Sodium | |
| | Total and direct bilirubin | |
| | Total protein | |
| | Cholesterol (total, HDL, LDL, VLDL) | |
| | Triglycerides | |

This 8 point ordinal scale as listed below is used to assess the severity of COVID-19 on given days during the study.

| Patient State | Descriptor | Score |
|---|---|---|
| Ambulatory | Asymptomatic | 0* |
| Ambulatory | Symptomatic, mild | 1 |
| | (no limitation of daily activities) | |
| Ambulatory | Symptomatic, moderate | 2 |
| | (limitation in daily activities) | |
| Hospitalized- mild | No O2 therapy | 3 |
| Hospitalized- mild | O2 by mask/nasal | 4 |
| Hospitalized -severe | Non-invasive ventilation | 5 |
| Hospitalized -severe | Mechanical Ventilation | 6 |
| Hospitalised -severe | Mechanical Ventilation + | 7 |
| | additional support | |
| Death | Death | 8 |

*Modified for this trial as infected, but without symptoms

Concomitant Medications and Other Therapy

Use of pioglitazone and rosiglitazone are strictly prohibited during the trial.

Use of other prescription or over-the-counter (OTC) medications or herbal remedies is allowed during the study, as long as the subject has been stabilized on a dose for 6 weeks prior to study entry. Any concomitant medication taken during the study must be recorded in the eCRF.

MSDC-0602K is metabolized to its hydroxy metabolite (MSDC-0597) by a carbonyl reductase, a ubiquitous enzyme, and there is no evidence to indicate that any concomitant medications would interfere with this metabolism. MSDC-0597 is a weak inhibitor of CYP2C8. Therefore, caution should be exercised when MSDC-0602K is administered with other therapeutics that are sensitive substrates for CYP2C8 (e.g., daprobustat, dasabuvi, repaglinide, paclitaxel) with the last two specifically prohibited during screening or during the study. Both MSDC-0602 and MSDC 0597 are inhibitors of organic anion transporter 3 (OAT3) so caution should be exercised when MSDC-0602 is co-administered with substrates for OAT3 (e.g., adefovir, cefaclor, ceftizoxime, famotidine, furosemide, methotrexate, oseltamivir carboxylate, penicillin G). MSDC-0602 is an inhibitor of MDR1 and BCRP and is therefore expected to inhibit intestinal multidrug resistance protein 1 (MDR1) and breast cancer-related protein (BCRP). As such, MSDC-0602 may cause an increase in systemic exposure to other therapeutic agents that are substrates for MDR1 or BCRP, if orally co-administered with MSDC-0602. Therefore, caution should be exercised with orally co-administered BCRP (methotrexate, imatinib, lapatinib, rosuvastatin and sulfasalazine) and MDR1 (digoxin, dabigatran etexilate, fexofenadine) substrates.

Physical Activity

Subjects will abstain from strenuous exercise other than normal activity (e.g., heavy lifting, weight training, calisthenics, aerobics) for 48 hours prior to each blood collection for clinical laboratory tests. Walking at a normal pace will be permitted.

Meals and Fluid Intake

Subjects must abstain from all food and drink (except water) at least 8 hours prior to any safety laboratory evaluations.

Contraception

Male or female subjects with reproductive potential must agree to either practice abstinence or comply with approved double barrier contraceptive method (e.g., condom plus IUD, condom plus hormonal contraception, condom and diaphragm with spermicidal gel or foam) during screening and for the duration of the trial.

Subjects are considered of nonreproductive potential if:

Postmenopausal female with ≥12 consecutive months of spontaneous amenorrhea and age≥55 years at Screening Visit.

Surgically sterile female and at least 6 weeks post-sterilization (i.e., bilateral tubal ligation, bilateral oophorectomy, or hysterectomy).

Sterilized Male at Least 1-Year Post-Vasectomy

Sperm or egg donation are both prohibited for the duration of the trial.

Early Termination of Study/Closure of Site

The study may be terminated early if new toxicological findings or results affecting the safety of the subjects become available. The sponsor as well as the investigators reserve the right to terminate the study at any time for any reason.

A site may be closed based on issues identified with subject recruitment, GCP compliance, poor quality data, evidence of attempted or proven fraud, or for any reason at the sponsor's discretion.

In the event the sponsor terminates a particular study site prior to the end of the trial, the site staff will promptly notify the Institutional Review Board (IRB)/Ethics Committee (EC).

Study Drug

Investigational and Control Drugs

The sponsor will provide the following double-blind study drugs:

MSDC-0602K 250 mg tablets

Matching Placebo tablets

MSDC-0602K tablets and placebo tablets will be packaged into 33 count bottles. On bottle will be assigned at visit 1 and two bottles will be assigned at visit 3.

Treatments

At Visit 1, all eligible subjects will be randomized in a 1:1 allocation ratio to one of the 2 treatment arms listed below:

MSDC-0602K 250 mg tablets

Matching Placebo tablets

Subjects will be instructed to take 1 tablet daily by mouth with water at least 30 minutes prior to a meal for the duration of the study. The subject should be instructed to swallow the tablet whole, and not to chew, cut, or crush the tablet.

Randomization Code Creation and Storage

Randomization personnel of the sponsor or designee will generate the randomization schedule. All randomization information will be securely stored, accessible only by authorized personnel.

Study Drug Blinding

The study is double-blind in that neither the subjects nor the investigator will be aware of the treatment administered. Blinding will be maintained throughout the study by use of active and placebo tablets of similar appearance. The series of random numbers from which the randomization algorithm operates will be created by personnel who will have no involvement in the day-to-day operations of the study. The random number series will be sequestered from all blinded study personnel, assuring there will be no unblinding information available to them until the study completes.

Study Drug Assignment and Dispensing Procedures

Study Drug Assignment

At Visit 1 (Randomization Visit), subjects who fulfill all the inclusion/exclusion criteria will be randomized to one of the two treatment arms via the IWRS. Each subject will be uniquely identified in the study by a subject ID number comprised of a combination of his/her site number and subject number. Once assigned to a subject, the subject ID number will not be reused. At Visit 2, one bottle of study drug will be dispensed via the IWRS system A separate User Manual will be provided with details for use of the IWRS.

Subjects, investigator staff, and persons performing the assessments will remain blinded to the identity of the treatment from the time of randomization until database lock, using the following methods:

Randomization data are kept strictly confidential until the time of unblinding and will not be accessible by anyone involved in the day-to-day conduct of the study (except for emergency unblinding as noted in Section 7.9).

The identity of the treatments will be concealed by the use of study drugs that are identical in packaging, labeling, and schedule of administration, appearance, taste, and odor.

The IWRS will maintain a record that identifies each subject and the treatment/study drug of an individual subject's treatment assignment. Unblinding of all subjects' treatment assignments will occur at the conclusion of the trial following final database lock.

Dispensing the Study Drug

Study site will be supplied by sponsor or designee with study drug in identically appearing packaging. The clinical supplies will be packaged and labeled in a double-blind fashion. Each bottle will contain 33 tablets. One bottle will be dispensed to subjects at Visit 2.

Instructions will be provided to advise the site on the proper method of randomizing the subject in the IWRS and selecting the correct medication ID numbered bottle for dispensing to a given subject at Visit 2.

Subjects will be instructed to self-administer study drug once daily with water at least 30 minutes before a meal. They will be instructed to swallow the drug whole and not to chew, cut, or crush the tablet.

Study Drug Interruptions

Treatment interruptions (3 or more consecutive doses) should be avoided. A drug interruption not due to a safety concern occurring at any time during any period will be considered a protocol deviation. An occasional missed tablet is not considered treatment interruption. All changes will be recorded in the study records.

Compliance

Treatment compliance will be assessed by monitoring drug accountability. The subject will be asked to return all used, partly used, and unused study medication bottles at Visit 4 and Visit 6/EOT. The investigator will compare the amount of study drug returned with the amount dispensed and question the subject in case of discrepancies. If warranted, the investigator will remind the subject of the importance of taking the study medication as prescribed. These discussions should be documented in the subject's medical record.

Study Drug Supply, Storage, and Tracking

Study drugs must be received by a designated person at the study site, handled and stored safely and properly, and kept in a secured location to which only the investigator and designated site staff have access. Upon receipt, all study drugs should be stored according to the instructions specified on the drug labels. Study drug must be kept in a secure cabinet or room with access restricted to only necessary study site personnel until it is used. Unused medication may be destroyed onsite with sponsor's approval or returned to the sponsor or designee for destruction. Clinical supplies are to be dispensed only in accordance with the protocol.

Medication labels will comply with the legal requirements of the countries where the study is being conducted. They will include storage conditions for the drug, but no information about the subject. The investigator must maintain an accurate record of the receipt of shipments and dispensing of study drug in a drug accountability form. Monitoring of drug accountability will be performed by the monitor during site visits and at the completion of the trial.

At the conclusion of the study, and as appropriate during the course of the study, the investigator will dispose or return all used and unused study drug to the sponsor or designee only after accountability is performed by the study monitor.

Emergency Unblinding

Unblinding is restricted to emergency situations and should be used only under circumstances where knowledge of the treatment is necessary for the proper management of the subject. When possible, the investigator should attempt to contact the medical monitor, sponsor, or designee before unblinding a subject's treatment assignment. The randomization code can only be broken if an emergency situation arises that, in the investigator's opinion, requires knowledge of the drug product dosed for management of the emergency medical condition. To unblind a subject's treatment assignment, the investigator will access the unblinding module within the IWRS. Instructions for breaking the blind will be provided to the site. Unless discontinued for a safety reason, study drug may be continued following unblinding of the subject's treatment assignment.

Analysis of Key Efficacy Endpoints

Primary Efficacy Endpoints

Adiponectin

Low levels of adiponectin are indicative of inflamed, dysfunctional adipose tissue and treatment with MSDC-0602 should increase adiponectin levels during the time frame of this trial. The mean change from baseline endpoints will be analyzed using an Analysis of Covariance (ANCOVA) model and will summarized using mean, median, standard deviations, 25th and 75th percentiles, min, and max.

COVID-19 Severity

The primary endpoint of disease severity will use the modified WHO COVID-19 ordinal scale (range 0 to 8, with higher ranks representing higher severity) as measured on visit 4 (approximately 14 days) The primary endpoint of COVID-19 severity will be analyzed using an ordinal logistic regression model assuming proportional odds and adjusting for treatment, sex, presence of diabetes mellitus, BMI, and age. Analysis will be according to the treatment groups as randomized. The odds ratio and the corresponding 95% confidence interval and p-values will be provided. A secondary analysis of the primary endpoint will use a binary analysis approach (chi-square test for homogeneity of proportions), for severity scores>1 or <1.

Key Secondary Efficacy Endpoints

The secondary efficacy endpoints related to the COVID-19 severity score will also be analyzed using the same logistic regression models and adjusting for similar covariates as used for the primary endpoint. The odds ratio and the corresponding 95% confidence interval and p-values will be provided. A secondary analysis of these endpoints will also be conducted using a binary analysis approach (chi-square test for homogeneity of proportions). For continuous variables, the mean change from baseline endpoints will be analyzed using an Analysis of Covariance (ANCOVA) model. Continuous variables will be summarized using mean, median, standard deviations, 25th and 75th percentiles, min, and max. Categorical variables will be summarized using number and percentages. All analyses will be on the intent-to-treat population. All statistical tests and confidence intervals will be two-sided. These measurements will provide information for construction of post-hoc hypotheses about which clinical presentations (e.g., inflammatory markers, cell counts, markers of hemostasis) might be related to outcomes.

Key Exploratory Measurements

Flow cytometry and transcriptional analysis of PBMCs will be conducted to determine whether there are changes in these profiles that accompany extended consequences of viral respiratory infections in mice (36).

Subgroup Analysis

Subgroup analysis may include specific comparisons across age, metabolic condition, and sex.

Supportive Secondary Efficacy Endpoints

Various post-hoc analyses may include correlation of subsets of secondary and exploratory analyses with COVID-19 severity measures.

Sample Size and Power Calculations

The 250 mg MSDC-0602K group will be compared to the placebo group at the two-sided 0.05 significance level. In earlier phase 2 trials at 28 days this exposure of MSDC-0602 produced a 146% increase in adiponectin levels versus a 4% change in placebo. This would provide >90% power at an N of 20 subjects. However, the co-primary endpoint in this trial is to determine the potential of this improved metabolic function to limit severity of the COVID-19 disease. Given that the relative risk of disease severity is on the order of 2-fold greater in the high-risk patient category versus those without these risks, we estimate that an N of 300 (150/group) would provide an approximately 80% power to show a significant reduction in severity, if treatment were to reduce the risk to that seen in those with in the lower risk categories. These estimates are in line with the magnitude of effect demonstrated by another antidiabetic agent in a similar type of trial of hospitalized patients (31). The data accumulated in this Phase 2 trial will be used to power a confirmatory trial.

Safety Analysis

Adverse Events

Safety data will be summarized in the SAF. All AEs will be coded using the Medical Dictionary for Regulatory Authorities (MedDRA). Treatment-emergent AEs are those with an onset after the first dose of study drug or any event already present that worsens in either intensity or frequency following exposure to the study treatment, through 7 days after permanent discontinuation of study drug. Adverse events with an onset between signing of informed consent and study drug initiation and those with an onset more than 7 days after the last dose of study drug will be listed separately. The incidence through study completion (end of Treatment Period) of TEAEs, serious TEAEs, TEAEs resulting in study drug discontinuation, TEAEs at least possibly related to study drug, and fatal SAEs will be summarized by system organ class and AE preferred term in each treatment group. A summary of TEAEs by preferred term and severity, using the worst reported severity grade for each event for a given subject, will be presented.

Clinical Laboratory

Descriptive statistics for central laboratory parameter values, and for values of changes from baseline, by treatment group and visit, will be provided. The occurrence of significantly abnormal changes in laboratory values from baseline will be summarized by treatment group. Graphical representations may also be presented.

Vital Signs

Descriptive statistics for vital signs values and for values of changes from baseline, by treatment group and visit, will be provided. The occurrence of significantly abnormal changes in laboratory values from baseline will be summarized by treatment group. Graphical representations may also be presented.

Demographic and Baseline Characteristics

Summary statistics will be provided by treatment group for demographics (e.g., age, sex, race, and ethnicity) and for baseline characteristics including medical history and randomization stratification factors.

Study Drug Exposure, Compliance, and Concomitant Therapies

A summary of the total follow-up time and the time on double-blinded study medication will be provided by treatment group.

Concomitant medication/therapy verbatim terms will be coded using the latest version of the World Health Organization (WHO) Drug Dictionary. The number and percentage of subjects taking concomitant medications will be summarized by Anatomic Therapeutic Chemical (ATC) classification and preferred term for each treatment group.

No formal statistical tests are planned for these variables.

Interim Analysis

No formal interim analysis is planned.

Biological Specimens

Blood samples will be collected, processed, stored, and shipped as outlined in respective Laboratory Manuals or instructions provided by responsible laboratories for clinical chemistry, hematology, and biomarkers.

It is the responsibility of the investigator to ensure that all personnel who will be handling, packaging, and/or shipping clinical specimens act in conformance with International Air Transport Association (IATA) regulations relating to the handling and shipping of hazardous goods.

Clinical and Laboratory Data Collection

Case Report Forms

An electronic version of the case report form (CRF) data collection form (eCRFs) for each subject will be provided. All appropriate subject data gathered during the study will be recorded in English on these forms.

Whenever possible, all information requested on a CRF sheet or entered directly into an eCRF should be completed. If information is not available, it should be documented as such. Paper based forms should be filled out with a black or blue ballpoint pen. All deletions and corrections must be made by drawing a single line through the error and writing the correct information next to the change. All corrections must be initialed and dated. Correction fluid must not be used. A tracking system for changes to the eCRFs (i.e., an audit system) will be available.

The completed CRFs or completed print out and/or electronic copy of the eCRFs for this study are the property of Cirius Therapeutics, Inc, and should not be made available to third parties, except for authorized representatives of appropriate health/regulatory authorities, without written permission from Cirius Therapeutics, Inc.

Laboratory Results

Laboratory tests (serum chemistry and hematology) will be analyzed by a central laboratory and reported to the clinical site as results are generated. The investigator will review and comment on any laboratory value reported outside alert ranges provided by the central laboratory. The laboratory report must be signed and kept in the study subject file at the site for the sponsor. The electronic copies of the laboratory data will represent the clinical chemistry and hematology source data.

Study Documentation and Records Retention

Access to Records

As required by the International Conference on Harmonization (ICH)-Good Clinical Practice (GCP) guidelines and regulatory authorities, the investigator will allow sponsor's representative(s) direct access to all pertinent medical records in order to allow for the verification of data gathered in the CRFs or the electronic data forms and for the review of the data collection process. The records, including source documentation, must also be available for inspection by relevant regulatory health authorities. Medical records may be accessed and reviewed on site or centrally/remotely.

Source Documents

Source documents may include, but are not limited to, questionnaires, laboratory reports, ECG tracings, x-rays, radiologist reports, biopsy slides or reports, ultrasound photographs, clinic notes or pharmacy records, and any other similar reports or records of any procedure performed in accordance with the protocol. Source documents may also include CRFs or electronic devices when information is recorded directly onto such forms or devices.

Whenever possible, the original recording of an observation should be retained as the source document; however, a photocopy is acceptable provided that it is a clear, legible, and exact duplication of the original document.

Record Retention (Investigator's Study File)

Government agency regulations and directives require that all study documentation pertaining to the conduct of a clinical study must be retained by the investigator. They shall be retained until at least 2 years after the last approval of a marketing application in an ICH region. The investigator may neither assign archiving of the files to someone else nor remove them to another location, without previously obtaining written approval from the sponsor. The sponsor will notify the investigator in writing when retention is no longer necessary.

1. A method of treatment, comprising administering to a subject in need thereof:
a therapeutically effective amount of a compound of structural Formula (I): 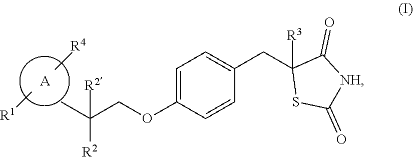
or a pharmaceutically acceptable salt thereof, wherein:
R¹ is hydrogen, halogen, substituted or unsubstituted alkyl, or —OR^{1A};
R² is halogen, hydroxyl, or optionally substituted aliphatic;
R^{2′} is hydrogen, or R² and R^{2′} may optionally be joined to form oxo;
R³ is hydrogen or deuterium;
R⁴ is hydrogen, halogen, substituted or unsubstituted alkyl, or —OR^{4A};
A is phenyl;
R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
wherein the subject has a metabolic disorder and a coronavirus infection.

2. (canceled)

3. The method of claim 1, wherein the metabolic disorder is a metabolic inflammation-mediated disease or disorder.

4. The method of claim 1, wherein the metabolic disorder comprises diabetes.

5. The method of claim 1, wherein the metabolic disorder comprises prediabetes.

6. The method of claim 4, wherein the diabetes comprises diabetes mellitus type II.

7. The method of claim 1, wherein the metabolic disorder comprises insulin resistance.

8. The method of claim 1, wherein the metabolic disorder comprises hyperinsulinemia.

9. The method of claim 1, wherein the metabolic disorder comprises glucose intolerance.

10. The method of claim 1, wherein the metabolic disorder comprises hyperglycemia.

11. (canceled)

12. The method of claim 1, wherein the coronavirus comprises severe acute respiratory syndrome coronavirus 2 (SARS-CoV-2).

13. The method of claim 12, wherein the coronavirus infection comprises coronavirus disease 2019 (COVID-19).

14. The method of claim 1, wherein the coronavirus comprises severe acute respiratory syndrome coronavirus (SARS-CoV).

15. The method of claim 1, wherein the coronavirus infection comprises severe acute respiratory syndrome (SARS).

16. (canceled)

17. The method of claim 1, wherein the coronavirus infection comprises Middle East respiratory syndrome (MERS).

18.-131. (canceled)

132. The method of claim 1, wherein R² and R^{2′} are joined to form oxo.

133. The method of claim 132, wherein the compound of structural Formula (I) is: 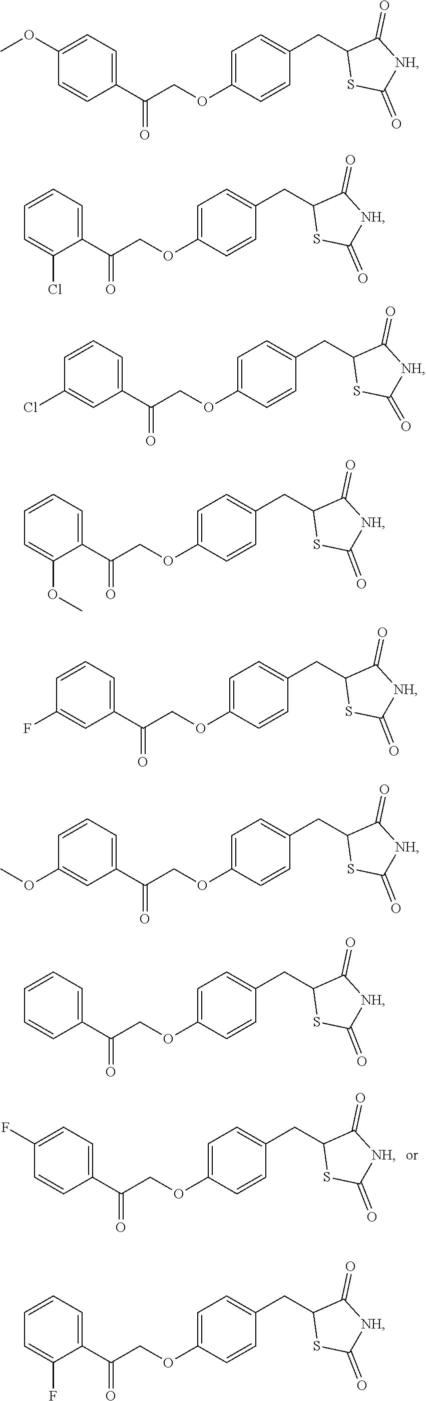
or a pharmaceutically acceptable salt thereof.

134. The method of claim 133, wherein the compound of structural Formula (I) is: 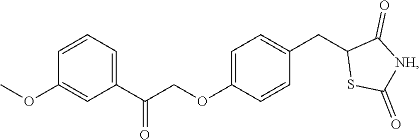 [SMILES: COc1cccc(C(=O)COc2ccc(CC3SC(=O)NC3=O)cc2)c1]
or a pharmaceutically acceptable salt thereof.

135.-200. (canceled)

201. The method of claim 1, wherein the therapeutically effective amount of the compound comprises a dosage amount of about 62.5 milligrams (mg), about 125 mg, or about 250 mg.

202. (canceled)

203. The method of claim 1, wherein the compound of structural Formula (I), or a pharmaceutically acceptable salt thereof, is administered in a dose of from about 60 mg to about 250 mg.

204.-205. (canceled)

206. The method of claim 134, wherein the pharmaceutically acceptable salt is a potassium salt.

207.-286. (canceled)
